(19) United States
(12) Patent Application Publication
(10) Pub. No.: US 2004/0071664 A1
(43) Pub. Date: Apr. 15, 2004

(54) Delivery of an agent

(75) Inventors: Anthony Patrick McHale, Coleraine (GB); Ana Maria Rollan-Haro, Madrid (ES); Roger Kingdon Craig, Sandbach (GB)

Correspondence Address: FROMMER LAWRENCE & HAUG, 745 FIFTH AVENUE- 10TH FL., NEW YORK, NY, 10151, US

(73) Assignee: Gendel Limited

(21) Appl. No.: 10/407,881

(22) Filed: Apr. 4, 2003

Related U.S. Application Data
(63) Continuation-in-part of application No. 09/748,063, filed on Dec. 22, 2000.
Application No. 09/748,063 is a continuation-in-part of application No. PCT/GB00/02848, filed on Jul. 24, 2000.
(60) Provisional application No. 60/146,556, filed on Jul. 30, 1999.

(30) Foreign Application Priority Data
Jul. 23, 1999 (GB) 9917416.1

(51) Int. Cl. A61K 45/00; A61N 1/00

(52) U.S. Cl. 424/93.7; 607/1

(57) ABSTRACT

The invention relates to a method for selectively releasing an agent loaded into a red blood cell, comprising electrosensitising the red blood cell by application of an electric field and subsequently disrupting the cell selectively using ultrasound.

### RELATED APPLICATIONS

This application is a continuation-in-part of U.S. patent application No. 09/748,063, filed Dec. 22, 2000, which claims the benefit of PCT/GB00/02848, filed Jul. 24, 2000 and to U.S. Provisional Application No. 60/146,556 filed Jul. 30, 2000 and to GB 9917416.1 filed Jul. 23, 1999.

### FIELD OF THE INVENTION

The present invention relates to a method for delivering an agent to a target site. In particular, the present invention relates to a method for delivering an agent in a red blood cell loaded with the agent, which cell is sensitised to assist in agent release. The present invention also relates to cells which are sensitised to a disrupting stimulus, such as ultrasound, and which can selectively release one or more agents loaded into the cells at a target site in vivo.

### BACKGROUND OF THE INVENTION

The delivery of a therapeutic agents to specific tissues is desirable typically to ensure that a sufficiently high dose of a given agent is delivered to a selected tissue. Moreover, it is often the case that the therapeutic agent, although advantageously having beneficial therapeutic effects on the diseased tissue, may have undesirable side effects on tissues that are not diseased. For example, in the treatment of certain types of disorders, such as cancer, it is necessary to use a high enough dose of a drug to kill the cancer cells without killing an unacceptable high number of normal cells. Thus, one of the major challenges of disease treatment is to identify ways of exploiting cellular drug delivery vehicles to incorporate and to selectively release agents at a desired target site.

It has been suggested that red blood cells may be exploited as active agent/drug delivery vehicles (DeLoach & Sprandel 1985, Bibliotheca Haematologica; Publ. Karger, Munich) as it is possible to incorporate agents into human red blood cells using a variety of techniques. An example of such a technique is the exploitation of osmotic shock and modifications thereof such as hypotonic shock and subsequent recovery of isotonicity and reverse hypotonic dialysis (Luque & Pinilla, 1993, Ind. Farmac. 8, 53-59).

An alternative method for loading drugs and active agents into red blood cells is electroporation. Using this process, the agent of interest are mixed with the live red blood cells in a buffer medium and short pulses of high electric fields are applied. The red blood cell membranes are transiently made porous and the agents of interest enter the cells. The electroporation process is advantageous as very high loading indices can be achieved within a very short time period (Flynn et al., 1994, Cancer Letts., 82, 225-229).

When packaging/carrier/delivery systems such as red blood cells are used as in vivo delivery systems, they suffer from the drawback that the delivery function is dependent upon both an accumulation of the red blood cells and a breakdown of the red blood cell membrane in or at the relevant tissue/site. As a result, attempts have been made to incorporate sensitising agents into cell carriers in order to facilitate both the accumulation and/or release of an agent of interest at a target site.

By way of example, our co-pending UK Patent Applications 9816583.0 9826676.0 relate inter alia to the incorporation of a dye compound, such as a porphyrin, which renders a loaded red blood cell susceptible to laser light treatment at a target site. This phenomenon, known as photodynamic activation, is exploited in order to achieve accumulation of the carrier vehicle at the relevant site and to achieve load release at that site.

Alternative energy sources have been investigated as tools for inducing payload release from loaded and sensitised cells. By way of example, ultrasound irradiation has been investigated as an alternative to light induced photodynamic activation as it has a broader degree of focus and it penetrates more deeply into the body. However, although ultrasound irradiation has also been applied to effect red blood cell lysis in vitro, its use has been limited in that its effect is only significant at lower cell concentrations (1-6×10⁶ cells) (Brayman et al., 1996, Ultrasound in Med & Biol., 22: 497-514). Moreover, ultrasound is non-specific in effects, resulting in lysis of both loaded and endogenous red blood cells.

Recently, it has been found that certain dye compounds, in particular porphyrins, can achieve a cytopathogenic effect when the disease site is subjected to ultrasound irradiation. This technique is referred to as sonodynamic therapy and is discussed in WO98/52609. WO98/52609 teaches that ultrasound irradiation may be useful in treating disease but only when it is combined with an effective amount of an ultrasound-susceptibility modification agent such as a porphyrin.

### SUMMARY OF THE INVENTION

The present invention provides a method for selectively releasing an agent from a loaded red blood cell at a target site.

According to a first aspect of the present invention, we provide the use of an electric field for sensitising a red blood cell to ultrasound.

Preferably, the electric field is used in a method which comprises the steps of: providing a red blood cell and subjecting the red blood cell to an electric field, the electric field having sufficient energy to electrosensitise the cell. More preferably, the red blood cell sensitised using electric field pulsing may be selectively disrupted using ultrasound.

According to a second aspect of the invention, we provide a method of selectively disrupting a red blood cell, the method comprising the steps of: (a) providing a red blood cell; (b) electrosensitising said red blood cell; and (c) disrupting said red blood cell by subjecting said red blood cell to ultrasound.

Preferably, the use according to the first aspect of the invention and the method according to the second aspect of the invention is such that the electrosensitisation comprises the step of applying an electric pulse to a red blood cell. Preferably, the electric pulse is from about 0.1 kVolts/cm to about 10 kVolts/cm under in vitro conditions.

The method or use according to the first and second aspects of the invention may further comprise the step of loading the red blood cell with an agent.

The sensitisation of the red blood cell may precede the loading of the agent. Alternatively, the loading of the agent precedes the sensitisation of the red blood cell. In yet another alternative, the sensitisation of the red blood cell and the loading of the agent are substantially simultaneous.

According to a third aspect of the invention, we provide a method for selectively releasing an agent from a red blood cell comprising the steps of: loading a red blood cell with an agent; electrosensitising the red blood cell; and causing the agent to be released from the electrosensitised red blood cell by applying ultrasound at a frequency and energy sufficient to cause disruption of the red blood cell but insufficient to cause disruption of unsensitised red blood cells.

According to a fourth aspect of the present invention, there is provided a method for delivering an agent to a target site in a vertebrate, comprising the steps of: providing a red blood cell; loading the red blood cell with an agent; electrosensitising the red blood cell; introducing the sensitised red blood cell into the vertebrate; and causing the disruption of the sensitised red blood cell by treatment of the cell with ultrasound to release the agent at a target site.

According to a fifth aspect of the present invention, there is provided a method for electrosensitising a red blood cell, comprising the steps of: providing a red blood cell; and subjecting the red blood cell to an electric field, the electric field having sufficient energy to electrosensitise the cell.

The electrosensitised red blood cells according to the invention may be loaded with agents either before, during or after the electrosensitisation procedure. In one aspect, therefore, the electrosensitisation procedure is effective to electroporate the cells, thus effecting simultaneous loading of a desired agent. Preferably, however, the electrosensitisation procedure is not effective to electroporate the cells, and the loading is thus carried out in a separate step either before or after the electrosensitisation procedure. Preferred methods for loading cells are set out below.

According to a sixth aspect of the invention, there is provided an electrosensitised red blood cell which is preparable by subjecting a red blood cell to an electric field at an energy level which is not effective to electroporate the cell. The invention also provides electrosensitised red blood cells according to the fourth aspect, which have been loaded with an agent using a process other than electroporation.

According to a seventh aspect of the present invention, there is provided a kit comprising a red blood cell, an agent, packaging materials therefor and instructions for use, the use comprising the steps of: electrosensitising a red blood; loading the red blood cell with an agent; causing the agent to be released from the electrosensitised red blood cell by exposure to ultrasound at a frequency and energy effective to cause disruption of the sensitised red blood cell but insufficient to cause disruption of unsensitised red blood cells.

According to a eighth aspect of the present invention, there is provided a kit comprising a red blood cell which is loaded with an agent, packaging materials therefor and instructions for use comprising the steps of: electrosensitising a red blood cell; and causing the agent to be released from the sensitised red blood cell by exposure to ultrasound at a frequency and energy effective to cause disruption of the sensitised red blood cell but insufficient to cause disruption of unsensitised red blood cells.

According to a ninth aspect of the present invention, there is provided a kit comprising a loaded electrosensitised red blood and instructions for causing the agent to be released from the electrosensitised red blood cell by exposure to ultrasound at a frequency and energy effective to cause disruption of the sensitised red blood cell but insufficient to cause disruption of unsensitised red blood cells.

In these and other aspects of the invention, the sensitisation and loading steps may be performed in any desired order, as appropriate.

### BRIEF DESCRIPTION OF THE FIGURES

FIG. 1 shows the effect of ultrasound power density on control normal (▪), electrosensitised normal (▴), control PEG-treated (▾), and electro-sensitised PEG-treated human red blood cells in PBS/Mg/glucose. X-axis: power density (W/cm²); left hand Y-axis: % lysis. The geometric mean (right-hand Y-axis) of fluorescence from populations of PEG-treated control (●) and electro-sensitised PEG-treated human red blood cells exposed to each power density and determined using flow cytometry are also plotted.

FIG. 2A shows the effect of ultrasound power density on control normal (●), control PEG-treated (▾), electro-sensitised (□) and electro-sensitised, PEG-treated (♦) human red blood cells in autologous plasma. X-axis: power density (W/cm²); Y-axis: % lysis.

FIG. 2B shows the geometric mean of fluorescence from populations of PEG-treated control (□) and electro-sensitised PEG-treated (▾) human red blood cells exposed to each power density and determined using flow cytometry. X-axis: power density (W/cm²); Y-axis: geometric mean.

FIG. 3 shows the effect of ultrasound (1.25W/cm², 30 seconds) on control normal (▪), control PEG-treated (▾), electro-sensitised normal (▴) and electro-sensitised PEG-(♦) human red blood cells which had been stored for the indicated times at 4° C. in PBS/Mg/glucose. X-axis: time (days); Y-axis: % lysis.

FIG. 4 shows the effect of ultrasound (1.25W/cm²) on control normal (▪), control PEG-treated (▴), electro-sensitised normal (▾) and electro-sensitised PEG-treated (♦) human red blood cells stored for the indicated times at 4° C. in autologous plasma. X-axis: time (days); Y-axis: % lysis.

FIG. 5 shows the effect of cell concentration on electro-sensitisation to ultrasound. Cells were (i) electro-sensitised at 8×10⁸ cells/ml, re-sealed in PBS/Mg, stored in PBS/Mg/glucose for 1 and 3 h and finally subjected to ultrasound (1.5 W/cm², 3MHz, 5 min.) (Samples 1 and 3, respectively). Alternatively (ii) cells were electro-sensitised at 14×10⁸ cells/ml and treated in a similar manner before treatment with ultrasound (Samples 2 and 4, respectively). Finally (iii) Sample 5 consisted of cells which were electro-sensitised at 8×10⁸ cells/ml, re-sealed as described above, pooled to 14×10⁸ cells/ml, stored for 3 h and finally treated with ultrasound. Control samples, C8, C14 and C14* consisted of cells treated in a similar manner to the three sets of cells described above (i, ii, iii, respectively) excluding electro-sensitisation. X-axis: sample number; Y-axis: % lysis.

FIG. 6 shows the response of electrosensitised and normal human red blood cells in a soft tissue mimicking phantom system following exposure to 5 min. ultrasound at 1.5 W/cm² and at 3 and 1 MHz. Cell samples were placed at an average distance of 1 cm from the scanning surface (ultrasound head surface) during treatments. Samples were then retrieved from the system and cell counts were determined using a hemocytometer. X-axis: sample number; Y-axis: % lysis.

FIG. 7 shows a flow cytometer analysis of cells subjected to loading by electroporation at varying electric field strengths. The filled traces represent cells which have not been electrosensitised, but exposed to antibody; these traces are overlayed with an open trace, representing cells which have been subjected to electroporation, with the antibody. The conditions are as follows: panel (a) 5×10⁷ RBC+0.5mg/ml antibody, pulsing twice at 1.45 KV 1 μF in PBS at 4 degrees C.; panel (b) 3.5×10⁷ RBC+0.25 mg/ml antibody, pulsing at 0.3 KV 10 μF, 1.45 KV 1 μF and 0.3 KV 10 μF in PBS at 4 degrees C.; pane (c) 4.5×10⁷ RBC+0.25 mg/ml antibody, pulsing at 0.3 KV 10 μF, 1.45 KV 1 μF and 0.3 KV 10 μF in PBSucrose at 4 degrees C. X-axis: FLH-1; Y-axis: counts.

FIG. 8 shows the effects of ultrasound on (i) cells treated with hypotonic dialysis (HD) (▪) and (ii) those treated with the hypotonic dialysis protocol, rested overnight at 4° C. and electro-sensitised (▴). X-axis: power density (W/cm²); Y-axis: % lysis.

FIG. 9 is a graph showing the ultrasound-mediated release of antibody from the erythrocyte vehicle. X-axis: power density (W/cm²), left hand Y-axis: μg of anti-vWF released (per 7×10⁷ cells treated by ultrasound); right hand Y-axis: percentage of cells lysed by ultrasound. Filled squares represent μg antibody, loaded cells; filled triangles represent μg antibody, control cells; open squares represent % lysis, loaded cells; open triangles represent % lysis, control cells.

FIG. 10 is a graph showing ultrasound mediated release of β-galactosidase from the erythrocyte vehicle. X-axis: power density (W/cm²), left hand Y-axis: percentage lysis; right hand Y-axis: % relative enzyme release. Filled squares represent control lysis, filled triangles represent sample lysis and filled diamonds represent sample release (i.e., release of enzyme).

FIG. 11 is a graph showing ultrasound-mediated release of oligonucleotide from the erythrocyte vehicle. X-axis: power density (W/cm²), left hand Y-axis: percentage cell lysis; right hand Y-axis: % oligonucleotide release. Filled squares represent control cell lysis, filled triangles represent cell lysis and filled diamonds represent % oligonucleotide released.

FIG. 12 shows ultrasound-mediated release of anti von Willebrand factor antibody from sensitised human erythrocytes in perfused rat kidney.

FIG. 13 shows gamma camera imaging of ⁹⁹Tc labelled electrosensitised (A), normal (B), glutaraldehyde-treated (C) and PEGylated (D) rabbit erythrocytes during circulation in a host rabbit. Images were captured over a 20 min. period at intervals. FIGS. 13A, B and C: first row 10″, 30″, 1′, 1′30″, 2′; second row 2′30″, 3′, 4′, 5′, 6′; third row 7′, 8′, 9′30″, 11′, 12′30″; fourth row 14′, 15′30″, 16′, 18′30″, 20′. FIG. 13D: row 1 10s, 20s, 30s, 40s, 50s, 1′, 70s, 80s; row 2 90s, 100s, 100 s, 2′, 130s, 140s, 150s, 160s; row 3 170s, 3′, 3′20s, 3′40s, 4′, 4′20s, 4′40s, 5′; row 4 5′20s, 5′40s, 6′, 6′20s, 6′40s, 7′, 7′20s, 7′40s; row 5 8′, 8.5′, 9′, 9.5′, 10′, 10.5′, 11′, 11.5′; row 6 12′, 12.5′, 13′, 13.5′, 14′, 14.5′, 15′, 15.5′; row 7 16′, 16.5′, 17′, 17.5′, 18′, 18.5′, 19′, 19.5′.

FIG. 14 shows clearance of ⁹⁹Tc-labelled normal (▴), PEGylated normal (▪) and PEGylated electrosensitised (♦) human erythrocytes during circulation in a recipient rabbit. X-axis: Time (minutes), Y-axis: %circulating cells.

FIG. 15 shows in vivo survival of PKH-26 labelled, normal autologous, normal heterologous and electrosensitised rabbit erythrocytes in recipient rabbits. X-axis: time post-injection (days); Y-axis % of PKH-26 labelled cells remaining in circulation. Normal human erythrocytes are included as a control for sequestration by the reticulo-endothelial system.-(continuous) unmodified autologous rabbit RBC; - - - unmodified heterologous rabbit RBC; - - - electrosensitised autologous rabbit RBC; - -- - -- unmodified human RBC; * predicted survival of rabbit RBC after 42 days in circulation.

FIG. 16 shows survival of PKH-26-labelled antibody-loaded and sensitised rabbit erythrocytes in vivo. X-axis: time (min); Y-axis: counts of labelled cells. The base line indicated by ▴ is for reference purposes and indicates the level of counts detectable prior to introduction of the labelled erythrocytes. Filled squares represent loaded and sensitised rabbit erythocytes.

FIG. 17 shows ultrasound-mediated release of antibody payload (anti-von Willebrand factor antibody) from loaded and sensitised human cells diluted in normal human cells at 40% hematocrit. Continuous wave ultrasound at 5W/cm² is used. X-axis: ultrasound exposure time (minutes); Y-axis: antibody payload released (%). The target cells were circulated through a system in which the temperature was maintained at 37° C. and the flow rate during exposure was 14.5 ml/min. Gray bars: control; black bars: test.

FIG. 18 shows clearance of rabbit-anti human IgG from rabbit circulation (n=3) as measured by ELISA. X-axis: time(days); Y-axis: antibody concentration in micrograms/ml.

FIG. 19 shows ultrasound-mediated release of rabbit anti-human IgG from loaded and sensitised rabbit erytlirocytes following exposure to ultrasound during circulation in vivo. X-axis: time (mins); Y-axis: antibody concentration in micrograms/ml. Filled squares (continuous -): test; filled upright triangles (black dashed -): control; filled inverted triangles (grey dashed -) represent pre-injection signal×2; right-pointing arrows: ultrasound treatment periods (1 MHz probe, 4 W/cm², 4′).

FIG. 20A. Graph showing ultrasound mediated release of peptide payload in vivo. Arrows above denote 10 minute applications of ultrasound pulsed wave (35%) at 6 W/cm².

FIG. 20B. Effect of ultrasound in circulating phantom upon electrosensitised loaded cells recovered from pig 10 minutes post administration. X-axis: time in circulating phantom at 6 W/cm²; Y-axis: cells in M1 region (loaded vehicle).

FIG. 21. Graph showing the in vivo effect of ultrasound on TAT-FITC loaded pig red blood cells, not electrosensitised. X-axis: time in minutes (ultrasound applications of 3×10 minute bursts at 6 W/cm² are indicated by downward arrows). Y-axis: number of cells in the fluorescent region.

FIG. 22A. Graph showing the in vivo effect of ultrasound on TAT-FITC loaded pig electrosensitised red blood cells. X-axis: time in minutes (ultrasound applications of 8×1 minute bursts at 6 W/cm² pulsed wave are indicated by downward arrows). Y-axis: number of fluorescent cells in the M4 region (i.e., loaded vehicle).

FIG. 22B. Graph showing the in vivo effect of ultrasound on TAT-FITC loaded electrosensitised pig red blood cells (enlargement of circled section in FIG. 22A). X-axis: time in minutes (ultrasound applications of 4×1 minute bursts at 6 W/cm² pulsed wave are indicated by downward arrows). Y-axis: number of fluorescent cells in the M4 region (i.e., loaded vehicle).

FIG. 23. Graph showing ultrasound mediated release of peptide payload in vivo in pig. X-axis: time in minutes. Y-axis geometric mean of the M2 region(i.e., loaded vehicle). Arrows indicate points when cells are administered and ultrasound applied to the hepatic artery region.

FIG. 24A. Graph showing ultrasound mediated changes in M4 cells (loaded vehicle) in vivo in pig. X-axis: time in minutes. Y-axis: events in region. Small arrows denote 30 second applications of ultrasound to the kidney; large arrows denote 1 minute applications of ultrasound to the kidney.

FIG. 24B. Ultrasound mediated localisation of FITC-labelled TAT in a treated kidney compared to a control untreated organ from the same animal (contralateral kidney). Upper panels: treated renal cortex (1), treated renal medulla (2); lower panels: control renal cortex (1), control renal medulla (2).

FIG. 25A shows graphs of experiments to establish optimal electrosensitisation cell density conditions for murine erythrocytes. Upper graph: electrosensitisation at 1×10⁹ cell density. X-axis: voltage in kV. Right hand Y-axis: % lysis with ultrasound. Left hand Y-axis: percentage recovery. Lower graph: electrosensitisation at 1.5×10⁹ cell density. X-axis: voltage in kV. Right hand Y-axis: % lysis with ultrasound. Left hand Y-axis: percentage recovery.

FIG. 25B shows graphs of experiments to establish optimal number of pulses during electrosensitisation of murine erythrocytes. Upper graph: electrosensitisation at 1×10⁹ cell density with one pulse. X-axis: voltage in kV. Right hand Y-axis: % sensitivity. Left hand Yaxis: percentage recovery. Lower graph: electrosensitisation at 1×10⁹ cell density with two pulses. X-axis: voltage in kV. Right hand Y-axis: % lysis with ultrasound. Left hand Y-axis: percentage recovery.

FIG. 25C is a flow cytometry profile showing dialysis loading of peptide into murine erythrocytes.

FIG. 26A shows the effects of ultrasound treatment on loaded mouse cells (M4) in circulating phantom. Mouse cells dialysis loaded with TAT-fragment are subjected to varying ultrasound intensities on the circulating phantom. X-axis: time in minutes. Y-axis: number of cells in M4 region. Filled squares: circulation only; inverted triangles: 4.5 W/cm²; filled diamonds: 5 W/cm²; circles: 6 W/cm²; upright triangles: 8 W/cm².

FIG. 26B shows haemoglobin release from electrosensitised, mouse cells dialysis loaded with TAT-fragment and subjected to varying ultrasound intensities in a circulating phantom system. X-axis: time in minutes. Y-axis: OD at 540 nm. Filled squares: circulation only; inverted triangles: 4.5 W/cm²; filled diamonds: 5 W/cm²; circles: 6 W/cm²; upright triangles: 8 W/cm².

FIG. 27A is a graph showing the effect of renal ultrasound treatment on the cell dynamics of loaded cells in a murine model. X-axis: time in minutes; Y-axis: percentage loaded cells. Filled squares: control percentage; upright triangles: ultrasound treated kidney percentages.

FIG. 27B shows the in vivo effects of ultrasound applied to mouse kidney, following administration of TAT-fragment loaded erythrocytes (approximately 13% spike) into a mouse. Upper panel: treated kidney; lower panel: untreated kidney.

FIGS. 28A and 28B. Binding of oligonucleotide, TAT and TAT-oligonucleotide conjugate to rabbit aorta, uptake of oligonucleotide, TAT and TAT-oligonucleotide conjugate by rabbit aorta. Samples of each species are placed in contact with the inner surface of rabbit aorta. Tissues are subsequently fixed and paraffin wax sections prepared. Samples are viewed using fluorescence microscopy (A,B & C) for the presence of TAT and with light microscopy (D,E & F) for the presence of biotinylated oligonucleotide. FIG. 28A Panel A: aorta+oligonucleotide no DAB, inner surface; Panel B: aorta+FITC-TAT-oligonucleotide-biotin conjugate, inner surface; Panel C: aorta+FITC TAT, inner surface: Panel D: aorta+biotin-oligonucleotide, inner surface. FIG. 28B Panel E: aorta+FITC-TAT oligonucleotide-biotin, inner surface; Panel F: aorta+FITC-TAT, inner surface.

FIG. 29. Flow cytometry profiles for control unloaded human erythrocytes and erythrocyte preparations loaded with the TAT-oligonucleotide conjugate.

FIG. 30. Uptake of TAT-oligonucleotide by inner surface of aorta following ultrasound mediated release from loaded human erythrocytes. Fluorescent images obtained from aorta samples placed in contact with PBS (A), TAT-oligonucleotide conjugate-containing lysates (B) and oligonucleotide-containing lysates (C). The latter two lysates are prepared by treating conjugate- and oligonucleotide-containing erythrocytes with ultrasound.

FIGS. 31A and 31B. Uptake of oligonucleotide and TAT-oligonucleotide conjugate by aorta following ultrasound mediated release from human erythrocytes. Light microscopy images obtained from aorta samples placed in contact with lysates containing oligonucleotide (A) and conjugate (B). Lysates are prepared by treating oligonucleotide- and conjugate-containing erythrocytes with ultrasound. Aorta samples are also placed in contact with untreated erythrocytes containing both oligonucleotide (C) and conjugate (D).

FIG. 32 is a schematic diagram for an experiment in which liver tissue samples are varying distances from a point of application of ultrasound are taken. 2 mm tissue samples (at 1 cm intervals from the treated area) are excised from the right medial lobe of the liver. The circle denotes the area of ultrasound treatment and the numbers indicate locations of tissue sampling. Samples are labelled L1, L2 etc. Corresponding samples from the right lateral lobe are used as a control. In addition, tissue samples are collected directly under the site of ultrasound treatment and at 0.5 cm depths into the organ, labelled L1A, L1B etc to enable a 2 dimensional profile of localised release.

FIG. 33 is a graph showing the effect of ultrasound targeting to the right medial lobe of pig liver, on TAT-FITC loaded cells in vivo. Ultrasound applied to the right medial lobe of the liver at 6 W/cm2, 35% pulsed wave. Small arrows denote 2.5 minute applications, medium sized arrows denote 5 minute applications, the leftmost two large arrows denote 10 minute applications, while the rightmost (red) arrow denotes a 20 minute application of ultrasound.

FIG. 34 are figures showing histopathological analysis on the ultrasound treated right medial lobe of the liver. Liver sections (as labelled) are visualised for fluorescent staining.

### DETAILED DESCRIPTION OF THE INVENTION

The present invention demonstrates the highly surprising findings that:

(i) exposure of red blood cells to electrosensitisation induces a hyper-sensitivity to ultrasound.

(ii) exposure of red blood cells to electrosensitisation induces a hyper-sensitivity to ultrasound without the addition of chemical agents.

(iii) exposure of red blood cells to electrosensitisation induces a hyper-sensitivity to ultrasound and allows the selective lysis of destabilised red blood cells with ultrasound with little or no effect on normal red blood cells under in vitro and in vivo conditions.

(iv) the present invention allows for the targeted delivery of an agent to a tissue of interest in a vertebrate using sensitised red blood cells which have no particular affinity for the target tissue. This is of particular importance where the target tissue is of a type which is widely distributed throughout the body (for example, skeletal muscle).

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning. A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing. Essential Techniques, John Wiley & Sons; J. M. Polak and James O'D. McGee, 1990, In Situ Hybridization. Principles and Practice; Oxford University Press; M. J. Gait (Editor), 1984, Oligonucleotide Synthesis. A Practical Approach, Irl Press; D. M. J. Lilley and J. E. Dahlberg, 1992, Methods of Enzymology. DNA Structure Part A. Synthesis and Physical Analysis of DNA Methods in Enzymology, Academic Press; Using Antibodies : A Laboratory Manual: Portable Protocol NO. I by Edward Harlow, David Lane, Ed Harlow (1999, Cold Spring Harbor Laboratory Press, ISBN 0-87969-544-7); Antibodies : A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Electrosensitisation

The term “electrosensitisation” encompasses the destabilisation of cells without causing fatal damage to the cells. According to this method, a momentary exposure of a cell to a high electric field results in membrane destabilisation. The strength of the electric field is adjusted up or down depending upon the resilience or fragility, respectively, of the cells being loaded and the ionic strength of the medium in which the cells are suspended.

Electrosensitisation typically involves the use of electric fields which do not possess sufficient energy to electroporate the cells. Electroporation, which facilitates the passage of agents into the cell without significant loss of cellular contents or cell viability, is well known in the art, and apart from the energy levels involved is similar to electrosensitisation. Indeed, cells which are electroporated become electrosensitised. However, electrosensitisation may be carried out at energy levels which are insufficient to electroporate the cell and permit the passage of substances through the cell wall. Thus, the invention encompasses the use of an electric field for sensitising a red blood cell to ultrasound.

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

These known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000V/cm, of about 100 μs duration. Such a pulse may be generated, for example, in known applications of the Electro Square Porator T820.

Electrosensitisation may be performed in a manner substantially identical to the procedure followed for electroporation, with the exception that lower electric field strengths may be used, as set forth below.

In a preferred aspect of the present invention, the electric field has a strength of from about 0.1 kVolts /cm to about 10 kVolts/cm under in vitro conditions.

Preferably the electric field has a strength of from about 1.5 kVolts/cm to about 4.0 kVolts/cm under in vitro conditions.

Preferably the electric field has a strength of from about 0.1 kVolts/cm to about 10 kVolts/cm under in vivo conditions (see WO97/49450).

Preferably the application of the electric field comprises multiple pulses.

Preferably the application of the electric field comprises sequential pulses (see Table 1).

Preferably the application of the electric field comprises double pulses.

Preferably the electric pulse is delivered as an exponential wave form.

Preferably the electric pulse is delivered as a square wave form.

Preferably the electric pulse is delivered as a modulated wave form.

As used herein, the term “electric pulse” includes one or more pulses at variable capacitance and voltage and including exponential and/or square wave and/or modulated wave forms.

Other electroporation procedures and methods employing electroporation devices are widely used in cell culture, and appropriate instrumentation is well known in the art.

Loading

As used herein, the term “loading” refers to a red blood cell which comprises at least one agent. The agent may be loaded by becoming internalised by, affixed to the surface of, or anchored into the plasma membrane of a red blood cell. Where the agent is affixed or anchored to the plasma membrane, loading may be achieved by cross-linking the agent to any cell surface molecule. Alternatively, the agent may be conjugated to or fused with an antibody specific for a cell surface molecule.

Loading of a red blood cell with more than one agent may be performed such that the agents are loaded individually (in sequence) or together (simultaneously or concurrently) and/or prior to, simultaneous with, sequential to or separate from, the “sensitising” procedure. The agents may be first admixed at the time of contact with the red blood cells or prior to that time.

According to the present invention, red blood cells may be loaded either prior to, simultaneously with, or after the sensitisation procedure. In one embodiment of the present invention, the red blood cells may be pre-loaded with the desired agent, and subsequently electrosensitised. In this embodiment, the loading may be performed by any desired technique. If they are loaded and sensitised substantially simultaneously, they may be loaded and sensitised by the same technique. Alternatively, the red blood cells may be sensitised and subsequently loaded. By way of example, the red blood cell may be sensitised by electrosensitisation, and loaded using osmotic shock or using electroporation. If more than one agent is employed, the same or a different technique may be used to load the second agent into the red blood cell. In general, if loading is subsequent to sensitisation, two or more agents can be loaded in any order. If loading is simultaneous, two or more agents can be admixed prior to contact with the red blood cells or can be added separately, prior to or after the application of the loading procedure mediates uptake of the agents by the cell.

As used herein, the term “substantially simultaneous” means that the site and time of loading and sensitisation are such that the loading and sensitisation are achieved at approximately the same time.

Preferably the red blood cells of the present invention are sensitised and loaded (in any order) in vitro or ex-vivo.

Preferably the loading is performed by a procedure selected from the group consisting of electroporation, sonoporation, microinjection, calcium precipitation, membrane intercalation, microparticle bombardment, lipid-mediated transfection, viral infection, osmosis, osmotic pulsing, osmotic shock, diffusion, endocytosis, phagocytosis, crosslinking to a red blood cell surface component, chemical crosslinking, mechanical perforation/restoration of the plasma membrane by shearing, single-cell injection or a combination thereof. Sonoporation as a method for loading an agent into a cell is disclosed in, for example, Miller et al (1998), Ultrasonics 36, 947-952.

Loading may also be achieved by modifying the environment of the red blood cell in such a way as to enable an agent to enter the red blood cell. For example, heat may be applied to the red blood cell; the effect of heat may be to make the red blood cell membrane more porous to external agent. The ionic strength of the environment of the red blood cell may also be modulated to encourage agent loading. Furthermore, the pH (or acidity, or hydrogen ion concentration) of the environment of the red blood cell may be altered to encourage loading. Radiation, for example, electromagnetic radiation, including infrared, visible light, X-rays, gamma rays, and ultraviolet rays, may be applied to the red blood cell to enable loading. Any one or more of the loading processes set forth in this document (including modulating the pH, ionic strength and/or applying heat) may be conducted in combination to encourage or cause loading of the agent into the red blood cell.

In a one aspect of the present invention, the loading procedure is carried out by iontophoresis.

Iontophoresis uses electrical current to activate and to modulate the diffusion of a charged molecule across a biological membrane, such as the skin, in a manner similar to passive diffusion under a concentration gradient, but at a facilitated rate. In general, iontophoresis technology uses an electrical potential or current across a semipermeable barrier. By way of example, delivery of heparin molecules to patients has been shown using iontophoresis, a technique which uses low current (d.c.) to drive charged species into the arterial wall. The iontophoresis technology and references relating thereto is disclosed in WO 97/49450.

In a further aspect of the present invention, loading is carried out by an osmotic shock procedure.

In more detail, the “osmotic shock” mechanism is taught in U.S. Pat. No. 4,478,824. That method involves incubating a packed red blood cell fraction in a solution containing a compound (such as dimethyl sulphoxide (DMSO) or glycerol) which readily diffuses into and out of cells, rapidly creating a transmembrane osmotic gradient by diluting the suspension of red blood cell in the solution with a near-isotonic aqueous medium. This medium contains an anionic agent to be introduced (such as a phosphorylated inositol) which may be an allosteric effector of haemoglobin, thereby causing diffusion of water into the cells with consequent swelling thereof and increase in permeability of the outer membranes of the cells. This increase in permeability is maintained for a period of time sufficient only to permit transport of the anionic agent into the cells and diffusion of the readily-diffusing compound out of the cells. This method is of limited effectiveness where the desired agent to be loaded into cells is not anionic, or is anionic or polyanionic but is not present in the near-isotonic aqueous medium in sufficient concentration to cause the needed increase in cell permeability without cell destruction.

U.S. Pat. No. 4,931,276 and WO 91/16080 disclose methods of loading red blood cells with selected agents using an osmotic shock technique. Therefore, these techniques can be used to enable loading of red blood cells in the present invention. In U.S. Pat. No. 4,931,276, a modified osmotic shock technique is provided.

Effective agents which may advantageously be loaded into red blood cells using the modified method provided in U.S. Pat. No. 4,931,276 include peptides, purine analogues, pyrimidine analogues, chemotherapeutic agents and antibiotic agents. These agents frequently present drug delivery problems. Specific compounds include but are not limited to tryptophan, phenylalanine and other water-soluble amino acid compounds. Several derivatives of the unnatural analogues of the nucleic acid bases adenine, guanine, cytosine and thymine are well known as useful therapeutic agents, e.g. 6-mercaptopurine (6MP) and azathioprine, which are commonly used as immunosuppressants and inhibitors of malignant cell growth, and azidothymidine (AZT) and analogues thereof which are useful as anti-viral agents, particularly in the treatment of AIDS. It has been shown that the action of these unnatural base derivatives is dependent on intra-cellular conversion thereof to phosphorylated forms (Chan et al., 1987, Pharmacotherapy, 7: 165;14 177; also Mitsuya et al., 1986, Proc. Natl. Acad. Sci. U.S.A., 83: 1911-1915).

An alternative osmotic shock procedure is described in U.S. Pat. No. 4,931,276 which is incorporated herein by reference.

In an advantageous aspect of the present invention, loading is carried out by a microparticle bombardment procedure.

Microparticle bombardment entails coating gold particles with the agent to be loaded, dusting the particles onto a 22 calibre bullet, and firing the bullet into a restraining shield made of a bullet-proof material and having a hole smaller than the diameter of the bullet, such that the gold particles continue in motion toward cells in vitro and, upon contacting these cells, perforate them and deliver the payload to the cell cytoplasm.

It will be appreciated by one skilled in the art that combinations of methods may be used to facilitate the loading of a red blood cell with agents of interest according to the invention. Likewise, it will be appreciated that a first and second agent, may be loaded concurrently or sequentially, in either order, into a red blood cell in any method of the present invention.

In further aspects, loading is carried out by hypotonic dialysis, also known as hypoosmotic dialysis. Protocols for hypotonic dialysis are known in the art, and are also described in detail in the Examples.

As would be apparent to one of skill in the art, any one or more of the above techniques can be used to load red blood cells for use in the invention, either prior to, simultaneously with, separate from or in sequence to the sensitisation procedure. For example, U.S. Pat. No. 4,224,313 discloses a process for preparing a mass of loaded cells suspended in a solution by increasing the permeability of the cell membranes by osmotic pressure or an electric field, or both, loading agents by passage from a solution through the membranes of increased permeability, restoring the original permeability by sealing the membranes by regeneration effect, and separating the cells from the solution in which they were suspended. In that procedure, the agents in solution which are to be loaded include i) a pharmaceutical substance which reacts chemically or physically with substances in the extracellular milieu and which, when loaded into the cell, would prematurely destroy the cell membranes, and ii) at least one blood-compatible sugar and protein capable of providing hydrogen bridge bonding- or of entering into covalent bonds with the pharmaceutical substance, thereby inhibiting the reaction of the pharmaceutical substance with the cell membranes.

Selective Release Using Ultrasound

According to the invention, agents which are loaded into a red blood cell are released from the red blood cell and into their surroundings, in this case at or into the target site, tissue or cell, by the application of ultrasound directed at a target site, tissue and/or cell.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′. (From Ultrasonics in clinical diagnosis. Edited by PNT Wells, 2 nd. Edition, Publ. Churchill Livingstone [Edinburgh, London & N.Y., 1977]. The term “ultrasound” includes diagnostic, therapeutic and focused ultrasound. Diagnostic ultrasound refers to an ultrasound energy source in a range up to about 100 mW/cm² (FDA recommendation). Therapeutic ultrasound refers to an ultrasound energy source in a range up to about 3-4 W/cm² (WHO recommendation).

Focused ultrasound (FUS) allows thermal energy to be delivered without an invasive probe (see Morocz et al 1998 Journal of Magnetic Resonance Imaging Vol.8, No. 1, pp. 136-142. Another form of focused ultrasound is high intensity focused ultrasound (HIFU) which is reviewed by Moussatov et al in Ultrasonics 1998 Vol.36, No.8, pp.893-900 and TranHuuHue et al in Acustica, 1997, Vol.83, No.6, pp. 1103-1106.

Preferably, a combination of diagnostic ultrasound and a therapeutic ultrasound is employed.

Preferably the ultrasound is applied to a target cell or target tissue with sufficient strength to disrupt loaded and sensitised red blood cells but without damaging the target tissue or surrounding tissues. In the context of the present invention, the term “damage or damaging” does not include a transient permeabilisation of the target site by the ultrasound energy source. Such a permeabilisation may facilitate uptake of the released payload at the target site.

Preferably the exposure to an ultrasound energy source is at a power density of from about 0.05 to about 100 Wcm⁻².

Even more preferably, the exposure to an ultrasound energy source is at a power density of from about 1 to about 15 Wcm⁻².

Preferably the exposure to an ultrasound energy source is at a frequency of from about 0.015 to about 10.0 MHz. Preferably the exposure to an ultrasound energy source is at a frequency of from about 0.02 to about 5.0 Mhz. Preferably the exposure to an ultrasound energy source is at a frequency of from about 0.5 to about 3 Mhz.

Preferably the exposure is for periods of from about 10 milliseconds to about 60 minutes.

Preferably the exposure is for periods of from about 1 second to about 5 minutes.

Particularly preferably the patient is exposed to an ultrasound energy source at an acoustic power density of from about 0.05 Wcm⁻² to about 10 Wcm⁻² with a frequency ranging from about 0.015 to about 10 MHz (see WO 98/52609). However, alternatives are also possible, for example, exposure to an ultrasound energy source at an acoustic power density of above 100 Wcm⁻², but for reduced periods of time, for example, 1000 Wcm⁻² for periods in the millisecond range or less.

Use of ultrasound is advantageous as, like light, it can be focused accurately on a target. Moreover, ultrasound is advantageous as it has a broader degree of three-dimensional focus than a light energy source and is better suited to whole-tissue penetration (such as but not limited to a lobe of the liver) or whole organ (such as but not limited to the entire liver or an entire muscle, such as the heart) delivery of agents according to the present invention. In addition, ultrasound may induce a transient permeabilisation of the target site so that uptake of a released payload is facilitated at the target site. Another important advantage is that ultrasound is a non-invasive stimulus which is used in a wide variety of diagnostic and therapeutic applications. By way of example, ultrasound is well known in medical imaging techniques and, additionally, in orthopaedic therapy. Furthermore, instruments suitable for the application of ultrasound to a subject vertebrate are widely available and their use is well known in the art.

In methods of the invention, release of the agent is effected by exposure of red blood cells either in vitro or ex-vivo to an effective amount of a diagnostic ultrasound energy source or a therapeutic ultrasound energy source as described in U.S. Pat. No. 5,558,092 and WO94/28873. The agent, which is released from a red blood cell for use in the present invention may be referred to as the “payload” of that cell. The term “payload” does not refer to the naturally-occurring contents of a red blood cell.

Preferably the agent is released from the red blood cell by treatment of a target site, tissue or cell with ultrasound.

The selective release of the agent at the target site can be determined by observing a) the amount which has been released at the target site, tissue or cell and b) its effect on the target site, tissue or cell, the latter determining whether its delivery should increase, decrease or be discontinued.

Blood Cells

In one embodiment of the present invention, the red blood cells which may be loaded and administered to a vertebrate according to the invention are ideally obtained from the intended recipient individual prior to the procedure so as to ensure complete immunocompatibility. Alternatively, cells are obtained from a second individual of the same species as the recipient; in such a case, the second individual must share the blood type of the intended recipient or must have an immuno-neutral blood type, such as type O in humans. Alternatively, the red blood cell may have its immunological determinants masked by a substance such as PEG (see below).

As used herein, the term “red blood cell” refers to a living, enucleate red blood cell (i.e., a mature erythrocyte) of a vertebrate.

Preferably the red blood cell is a mammalian red blood cell, advantageously a human red blood cell. As used herein, the term “mammal” refers to a member of the class Mammalia including, but not limited to, a rodent, lagomorph, pig or primate. Preferably, the mammal is a human.

As used herein the term “introducing” includes but is not limited to the administration of a red blood cell and/or an agent into a vertebrate.

As used herein in reference to administration of an agent to a vertebrate, the term “introducing” includes but is not limited to causing the agent to enter the circulatory system of the vertebrate by transfusion or to infusing an agent to a target site. It is contemplated that a hollow needle, such as a hypodermic needle or cannula, is inserted through the wall of a blood vessel (e.g., a vein or artery) and the red blood cell is either injected using applied pressure or allowed to diffuse or otherwise migrate into the blood vessel. It is understood that the diameter of the needle is sufficiently large and the pressure sufficiently light to avoid damage of the cell by shear forces. Preferably, introduction of a red blood cell into a vertebrate in a method of the invention is intra-arterial or intravenous. Methods of blood cell transfusion are well known in the art.

Immunocompatibility

The loaded red blood cell vehicles may be coated with an agent which masks cell surface antigens. For example, PEGylated red blood cells evade the host immune response and thereby enjoy prolonged circulation. According to one such method, methoxy (polyethylene glycol), or mPEG, is covalently bound to red blood cells (Scott et al., 1997, Proc. Natl. Acad. Sci. U.S.A., 94: 7566-7571). This procedure has been shown to result in a loss of ABO blood group reactivity and inhibition of phagocytic destruction by monocytes; in addition, the survival of mPEG treated sheep red blood cells transfused into mice is increased 360-fold over that of untreated control cells (Scott et al., 1997, supra).

A second coating which may be useful in the invention is one which comprises distearoyl-phosphatidylethanolamine (DSPE)-conjugated PEG (Du et al., 1997, Biochim. Biophys. Acta—Biomembranes, 1326: 236-248). When applied as a monolayer film to a glass plate, DSPE-PEG inhibits protein adsorption and cell adhesion to the glass plate (Du et al., 1997, supra).

Targeting

According to a method disclosed in U.S. Pat. No. 4,669,481, limited targeting of red blood cells to a small subset of vertebrate tissues is achieved, if desired, as follows: Treating the red blood cell under mild heating conditions will damage the cells, resulting in rapid sequestration by the reticuloendothelial system. The cells can be specifically targeted for the spleen by heating for 10 minutes at 49° C. Greater temperature or length of heating produces increased cell damage, with resultant hepatic uptake. Thus, if desired, payload delivery to the spleen or liver can be preferentially enhanced; however, the degree to which the payload is lost from damaged cells prior to administration is not known.

As used herein, the term “target” is used in reference to the spatial coordinates (anatomical location) of the cell, tissue or site (such as a vessel) to which the agent of the present invention is delivered.

The red blood cells of the invention may be targeted to any desired site in a vertebrate, or mammal. As used herein, the term “site” refers to a region of the body of a vertebrate, which region may comprise an anatomical area, a tissue, a group of tissues, a cell, a group of cells or even substantially all of the cells of the vertebrate.

Preferably the target is a cell.

As used herein, the term “cell” refers to a viable, naturally-occurring or genetically engineered, single unit of an organism.

Preferably the target is a tissue.

As used herein, the term “tissue” refers to a population or physical aggregation of cells within an organism, wherein the cells are of the same cell type or are of cell different types resident within a single organ or other functional unit. As used herein, the term “tissue” refers to intact tissue or tissue fragments, such that the cells are sufficiently aggregated (associated) so as to form a cohesive mass. Alternatively, the term “tissue” refers to a collection of individual cells, such as those which circulate (e.g., in blood or lymphatic fluid) within the vertebrate. A tissue may comprise an entire organ (e.g. the pancreas, the thyroid, a muscle, bone or others) or other system (e.g. the lymphatic system) or a subset of the cells thereof, therefore, a tissue may comprise 0.1-10%, 20-50% or 50-100% of the organ or system (e.g., as is true of islets of the pancreas).

Preferably the target is a vessel.

As used herein the term “vessel” means any artery, vein or other “lumen” in an organism to which ultrasound can be applied and to and an agent may be delivered. A lumen is a channel within a tube or tubular organ. Examples of preferred vessels in the method of the present invention include but are not limited to the coronary artery, carotid artery, the femoral artery, and the iliac artery.

In one embodiment of the present invention, an ultrasound energy source may be focused at the target cell, tissue or site (such as a vessel) as loaded red blood cells circulate through it. For example, a diagnostic and/or therapeutic ultrasound energy source or a combination thereof may be applied to a target tissue. This is particularly applicable to target tissues located on the surface of the subject vertebrate, although deep targets may also be treated with an ultrasound energy source.

Agent

As used herein, the term “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid and a carbohydrate. An agent may be in solution or in suspension (e.g., in crystalline, colloidal or other particulate form). The agent may be in the form of a monomer, dimer, oligomer, etc, or otherwise in a complex.

The agent may be an imaging agent, by which term is meant an agent which may be detected, whether in vitro in the context of a tissue, organ or organism in which the agent is located. The imaging agent may emit a detectable signal, such as light or other electromagnetic radiation. The imaging agent may be a radio-isotope as known in the art, for example ³²P or ³⁵S or ⁹⁹Tc, or a molecule such as a nucleic acid, polypeptide, or other molecule as explained below conjugated with such a radio-isotope. The imaging agent may be opaque to radiation, such as X-ray radiation. The imaging agent may also comprise a targeting means by which it is directed to a particular cell, tissue, organ or other compartment within the body of an animal. For example, the agent may comprise a radiolabelled antibody specific for defined molecules, tissues or cells in an organism.

The imaging agent may be combined with, conjugated to, mixed with or combined with, any of the agents disclosed herein.

It will be appreciated that it is not necessary for a single agent to be used, and that it is possible to load two or more agents for into the vehicle. Accordingly, the term “agent” also includes mixtures, fusions, combinations and conjugates, of atoms, molecules etc as disclosed herein. For example, an agent may include but is not limited to: a nucleic acid combined with a polypeptide; two or more polypeptides conjugated to each other; a protein conjugated to a biologically active molecule (which may be a small molecule such as a prodrug); or a combination of a biologically active molecule with an imaging agent.

As used herein, the term “biological effector molecule” or “biologically active molecule” refers to an agent that has activity in a biological system, including, but not limited to, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. If the biological effector molecule is a polypeptide, it may be loaded directly into a red blood cell of the invention; alternatively, a nucleic acid molecule bearing a sequence encoding the polypeptide, which sequence is operatively linked to transcriptional and translational regulatory elements active in a cell at the target site, may be loaded. Small molecules, including inorganic and organic chemicals, are also of use in the present invention. In a particularly preferred embodiment of the invention, the biologically active molecule is a pharmaceutically active agent, for example, an isotope.

Particularly useful classes of biological effector molecules include, but are not limited to, antibiotics, anti-inflammatory drugs, angiogenic or vasoactive agents, growth factors and cytotoxic agents (e.g., tumour suppressers). Cytotoxic agents of use in the invention include, but are not limited to, diptheria toxin, Pseudomonas exotoxin, cholera toxin, pertussis toxin, and the prodrugs peptidyl-p-phenylenediamine-mustard, benzoic acid mustard glutamates, ganciclovir, 6-methoxypurine arabinonucleoside (araM), 5-fluorocytosine, glucose, hypoxanthine, methotrexate-alanine, N-[4-(a-D-galactopyranosyl) benyloxycarbonyl]-daunorubicin, amygdalin, azobenzene mustards, glutamyl p-phenylenediamine mustard, phenolmustard-glucuronide, epirubicin-glucuronide, vinca-cephalosporin,phenylenediamine mustard-cephalosporin, nitrogen-mustard-cephalosporin, phenolmustard phosphate, doxorubicin phosphate, mitomycin phosphate, etoposide phosphate, palytoxin-4-hydroxyphenyl-acetamide, doxorubicin-phenoxyacetamide, melphalan-phenoxyacetamide, cyclophosphamide, ifosfamide or analogues thereof . If a prodrug is loaded in inactive form, a second biological effector molecule may be loaded into the red blood cell of the present invention. Such a second biological effector molecule is usefully an activating polypeptide which converts the inactive prodrug to active drug form, and which activating polypeptide is selected from the group that includes, but is not limited to, viral thymidine kinase (encoded by Genbank Accession No. J02224), carboxypeptidase A (encoded by Genbank Accession No. M27717), α-galactosidase (encoded by Genbank Accession No. M 13571), β-glucuronidase (encoded by Genbank Accession No. M 15182), alkaline phosphatase (encoded by Genbank Accession No. J03252 J03512), or cytochrome P-450 (encoded by Genbank Accession No. D00003N00003), plasmin, carboxypeptidase G2, cytosine deaminase, glucose oxidase, xanthine oxidase, β-glucosidase, azoreductase, t-gutamyl transferase, β-lactamase, or penicillin amidase. Preferably, the polypeptide capable of activating a prodrug is DT diaphorase. Either the polypeptide or the gene encoding it may be loaded; if the latter, both the prodrug and the activating polypeptide may be encoded by genes on the same recombinant nucleic acid construct.

Preferably the biological effector molecule is selected from the group consisting of a protein, a polypeptide, a peptide, a nucleic acid, a virus, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

The present invention advantageously employs agents which are not able to diffuse through an intact erythrocyte cell wall by passive or active means. However, the delivery of agents which diffuse at a certain rate through the erythrocyte cell wall is contemplated, particularly where increased delivery of the agent at a particular time or location is desirable. Increased delivery may be achieved by ultrasound administation at the approriate time or location.

The agents, including biological effector molecules, may also be delivered into cells as fusions (for example, protein or polypeptide fusions) or conjugates with a protein capable of crossing the plasma membrane and/or the nuclear membrane. Preferably, the agent/biological effector molecule is fused or conjugated to a domain or sequence from such a protein responsible for the translocational activity. Preferred translocation domains and sequences include domains and sequences from the HIV-1-trans-activating protein (Tat), Drosophila Antennapedia homeodomain protein and the herpes simplex-1 virus VP22 protein. By this means, the agent/biological effector molecule is able to enter the cell or its nucleus when released in the vicinity of the cell using the methods described herein.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U. S. A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. Intraperitoneal injection of a fusion protein consisting of β-galactosidase and a HIV-TAT protein transduction domain results in delivery of the biologically active fusion protein to all tissues in mice (Schwarze et al., 1999, Science 285, 1569-72)

The third helix of the Drosophila Antennapedia homeodomain protein has also been shown to possess similar properties (reviewed in Prochiantz, A., 1999, Ann N Y Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chem, 269, 10444-50). This peptide has been used to direct biologically active substances to the cytoplasm and nucleus of cells in culture (Theodore, et al., 1995, J. Neurosci 15, 7158-7167). Cell internalization of the third helix of the Antennapedia homeodomain appears to be receptor-independent, and it has been suggested that the translocation process involves direct interactions with membrane phospholipids (Derossi et al., 1996, J Biol Chem, 271, 18188-93). The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives et al. (1997), J Biol Chem 272, 16010-7. Alternatively, green fluorescent protein may be used as a reporter (Phelan et al., 1998, Nat Biotechnol 16, 440-3).

Any of the domains or sequences or as set out above or identified as having translocational activity may be used to direct the agents (including biological effector molecules) into the cytoplasm or nucleus of a cell.

Nucleic Acid

A nucleic acid of use in the invention may comprise a viral or non-viral DNA or RNA vector, where non-viral vectors include, but are not limited to, plasmids, linear nucleic acid molecules, artificial chromosomes and episomal vectors. Expression of heterologous genes has been observed after injection of plasmid DNA into muscle (Wolff J. A. et al., 1990, Science, 247: 1465-1468; Carson D. A. et al., U.S. Pat. No. 5,580,859), thyroid (Sykes et al., 1994, Human Gene Ther., 5: 837-844), melanoma (Vile et al., 1993, Cancer Res., 53: 962-967), skin (Hengge et al., 1995, Nature Genet., 10: 161-166), liver (Hickman et al., 1994, Human Gene Therapy, 5: 1477-1483) and after exposure of airway epithelium (Meyer et al., 1995, Gene Therapy, 2: 450-460).

As used herein, the term “nucleic acid” is defined to encompass DNA and RNA or both synthetic and natural origin which DNA or RNA may contain modified or unmodified deoxy- or dideoxy-nucleotides or ribonucleotides or analogues thereof. The nucleic acid may exist as single- or double-stranded DNA or RNA, an RNA/DNA heteroduplex or an RNA/DNA copolymer, wherein the term “copolymer” refers to a single nucleic acid strand that comprises both ribonucleotides and deoxyribonucleotides. The term “nucleic acid” is also intended to include oligonucleotides and modified oligonucleotides.

The term “synthetic”, as used herein, is defined as that which is produced by in vitro chemical or enzymatic synthesis.

Therapeutic nucleic acid sequences useful according to the methods of the invention include those encoding receptors, enzymes, ligands, regulatory factors, and structural proteins. Therapeutic nucleic acid sequences also include sequences encoding nuclear proteins, cytoplasmic proteins, mitochondrial proteins, secreted proteins, plasmalemma-associated proteins, serum proteins, viral antigens, bacterial antigens, protozoal antigens and parasitic antigens. Therapeutic nucleic acid sequences useful according to the invention also include sequences encoding proteins, lipoproteins, glycoproteins, phosphoproteins and nucleic acids (e.g., RNAs such as ribozymes or antisense nucleic acids). Ribozymes of the hammerhead class are the smallest known, and lend themselves both to in vitro synthesis and delivery to cells (summarised by Sullivan, 1994, J. Invest. Dermatol., 103: 85S-98S; Usman et al., 1996, Curr. Opin. Struct. Biol., 6: 527-533). Proteins or polypeptides which can be expressed by nucleic acid molecules delivered according to the present invention include hormones, growth factors, neurotransmitters, enzymes, clotting factors, apolipoproteins, receptors, drugs, oncogenes, tumour antigens, tumour suppressers, structural proteins, viral antigens, parasitic antigens and bacterial antigens. The compounds which can be incorporated are only limited by the availability of the nucleic acid sequence encoding a given protein or polypeptide. One skilled in the art will readily recognise that as more proteins and polypeptides become identified, their corresponding genes can be cloned into the gene expression vector(s) of choice, administered to a tissue of a recipient patient or other vertebrate, and expressed in that tissue.

Delivery of Agents

The method of the present invention is useful for the delivery of agents to a selected site in a vertebrate body, whether an organ, part of an organ or otherwise, in the presence or absence of specific targeting means. This is achieved, as set out above, by the selective disruption by ultrasound at the selected target site of electrosensitised red blood cells loaded with the agent of choice.

Agents useful for use in the present invention are set out above. Preferred agents include those useful for imaging of tissues in vivo or ex vivo. For example, imaging agents, such as labelled antibodies which are specific for defined molecules, tissues or cells in an organism, may be used to image specific parts of the body by releasing them at a desired location using ultrasound. This allows imaging agents which are not completely specific for the desired target, and which might otherwise lead to more general imaging throughout the organism, to be used to image defined tissues or structures. For example, an antibody which is capable of imaging endothelial tissue may be used to image liver vasculature by releasing the antibody selectively in the liver by applying ultrasound thereto.

Kits

The invention also encompasses a kit comprising a red blood cell, an agent and packaging materials therefor.

A kit designed for the easy delivery of an agent to a recipient vertebrate, whether in a research of clinical setting, is encompassed by the present invention. A kit takes one of several forms, as follows:

A kit for the delivery of an agent to a subject vertebrate comprises red blood cells and the agent and instructions for performing the method of the present invention. Alternatively, the red blood cells are supplied loaded with the agent for convenience of use by the purchaser. The cells are supplied sensitised for rapid use or, for greater stability, unsensitised. In the latter case, the sensitising process is carried out separately from the cells. The cells of the kit are species-specific to the vertebrate of interest, such as a primate, including a human, canine, rodent, pig or other, as desired; in other words, the cells are of like species with the intended recipient. The cells of the kit are, additionally, specific to the blood type of the intended recipient organism, as needed. Optionally, the kit comprises one or more buffers for cell sensitisation, washing, resuspension, dilution and/or administration to a vertebrate. Appropriate buffers are selected from the group that includes low ionic strength saline, physiological buffers such as PBS or Ringer's solution, cell culture medium and blood plasma or lymphatic fluid. The kit additionally comprises packaging materials (such as tubes, vials, bottles, or sealed bags or pouches) for each individual component and an outer packaging, such as a box, canister or cooler, which contains all of the components of the kit. The kit is shipped refrigerated. Optionally, non-cellular components are supplied at room temperature or frozen, as needed to maintain their activity during storage and shipping. They may be in liquid or dry (i.e., powder) form.

A second kit of the invention comprises an agent such as a biological effector molecule, instructions for performing the method of the present invention and, optionally a sensitising device and buffers therefor (e.g., saline or other physiological salt buffer, culture medium, plasma or lymphatic fluid). In addition, the kit contains appropriate packaging materials, as described above. The individual components may be supplied in liquid or dry (i.e., powder) form, and may be at room temperature, refrigerated or frozen as needed to maintain their activity during storage and shipping. Red blood cells for use with this kit are obtained independently (for example, they may be harvested from the intended recipient vertebrate).

A preferred aspect of the invention is a kit comprising a red blood cell which is loaded with an agent, and packaging materials therefor.

Preferably, a kit as described above further comprises an apparatus for applying the sensitising procedure.

Preferably the kit further comprises polyethylene glycol.

It is additionally preferred that the kit further comprises a liquid selected from the group consisting of a buffer, diluent or other excipient.

Preferably, the liquid is selected from the group consisting of a saline buffer, a physiological buffer and plasma. A final aspect of the invention is a physiological composition comprising a red blood cell comprising a biological effector molecule admixed with a physiologically compatible buffer. As used herein, the term “physiologically compatible buffer” or “physiological buffer” is defined as a liquid composition which, when placed in contact with living cells, permits the cells to remain alive over a period of minutes, hours or days. As such, a physiological buffer is substantially isotonic with the cell, such that cell volume does not change more than 20% due to differences in internal and external ionic strength. Non-limiting examples of physiologically compatible buffers or physiological buffers include dilute saline, which may be buffered (e.g., Hanks' buffered saline or phosphate buffered saline), or other physiological salts (e.g., Ringer's solution), dilute glucose, sucrose or other sugar, dilute glycerol with- or without salts or sugars, cell culture media as are known in the art, serum and plasma.

Preferably, the red blood cell of the physiological composition is human.

### EXAMPLES

### Example 1

### Electric Field-Mediated Sensitivity of Human Red Blood Cells and Polyethylene Glycol (PEG)-Treated Red Blood Cells to Ultrasound in PBS/Mg/Glucose Buffer

The responses of normal cells were compared with those of electrosensitised and re-sealed cells following exposure to ultrasound over a range of power densities. The responses of normal cells were also compared with those of polyethylene glycol (PEG) treated cells which had been electrosensitised and re-sealed under similar conditions. To this end human blood was harvested by venipuncture and washed twice in PBS (phosphate buffered saline) by centrifugation. Cells were suspended in PBS containing 1 mg/ml fluorescein to yield concentrations of 7×10⁸ cells/ml and 0.8 ml aliquots were dispensed into electroporation cuvettes (0.4 cm electrode gap) and retained on ice for 10 min. Cells were then exposed to an electroporation strategy involving delivery of two electric pulses (field strength=3.625 kV/cm at a capacitance of 1 μF) using a BioRad Gene Pulser apparatus. Cells were immediately washed with PBS containing MgCl₂ (4 mM) (PBS/Mg) and retained at room temperature for 30 min. in the PBS/Mg (containing 1 mg/ml fluorescein) buffer to facilitate re-sealing. Aliquots of those cells were treated with polyethylene glycol (av. mol. Weight=5000) as described by Scott et al., 1997 Proc. Natl. Acad. Sci. (U.S.A.), 94, 7566-7571 using cyanuric chloride treated methoxy polyethylene glycol at a concentration of 25 mg/ml. Cells were subsequently washed and suspended at a concentration of 14×10⁸ cells/ml in PBS/Mg containing 10 mM glucose (PBS/Mg/glucose) for at least 1 hour. Samples were treated with ultrasound by dispensing 0.1 ml aliquots of cells into microwells (inner diameter=5 mm). Ultrasound power densities were generated using a Rich-Mar Multi Hz generator fitted with a 3 MHz ultrasound head (U.S.A.) and set to delivery continuous wave ultrasound at the required power density. Samples were treated for 30 seconds and cell counts were subsequently determined using a haemocytometer. In addition, samples of cells were analysed using a Becton Dickinson flow cytometer in order to determine leakage of fluorescein following exposure to ultrasound.

Results 1

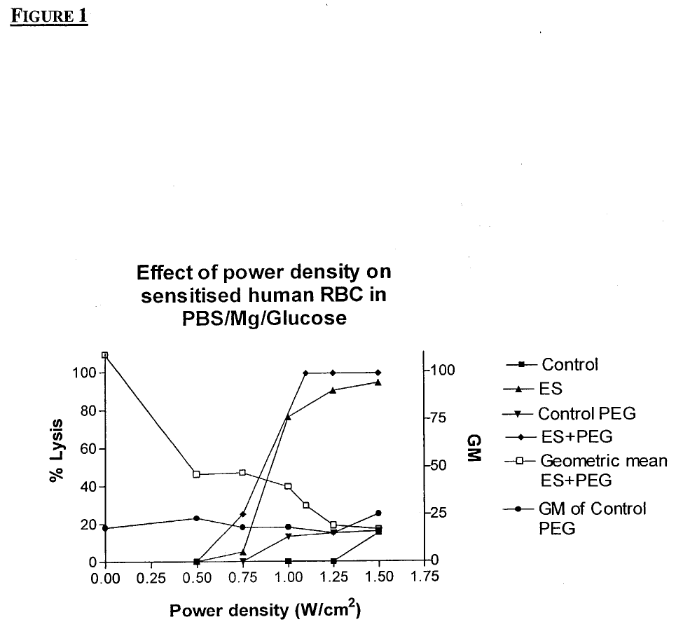

The results are shown in FIG. 1 and they demonstrate that the ultrasound power densities between 0.5 and 1.5 W/cm² had little or not effect on either normal or PEG-treated red blood cells. However, when electrosensitised normal and PEG-treated cells were exposed to increasing ultrasound power densities very significant lysis was detected, particularly at densities above 1 W/cm². The results demonstrate that cells which had been treated using conditions suitable for electroporative loading of materials into human red blood cells were rendered hyper-sensitive to ultrasound during that loading procedure. In addition to examining cell lysis it was also decided to employ flow cytometry to determine whether or not the pay-load was released during exposure to ultrasound. Again the results are shown in FIG. 1 and it was found that when PEG-treated cells loaded with fluorescein were exposed to increasing ultrasound power densities the geometric mean of fluorescence decreased. The control population for this experiment consisted of PEG-treated cells which were exposed to fluorescein in the absence of exposure to electric field conditions. The results demonstrate ultrasound-mediated leakage of a pay-load from the hyper-sensitised, PEG-treated red blood cells.

### Example 2

### Electric Field-Mediated Sensitivity of Human Red Blood Cell and Polyethylene Glycol-Treated Red Blood Cells to Ultrasound in Autologous Plasma

The data in Example 1 demonstrated that cells which had been treated with electric field conditions suitable for loading human red blood cells were rendered hyper-sensitive to ultrasound. This degree of sensitivity remained following storage of the samples for 1 hour in PBS/Mg/glucose buffer. It was of interest to determine whether or not the electro-sensitised cells exhibited sensitivity to ultrasound in the presence of autologous plasma. To this end both normal and PEG-treated human red blood cells were electro-sensitised in the presence of fluorescein as described for Example 1. Cells were allowed to re-seal in PBS/Mg (containing fluorescein) for 30min. and subsequently placed in PBS/Mg/glucose for 15 min. Cells were then suspended in autologous plasma at a concentration of approximately 14×10⁸ cell/ml. Cells were stored at room temperature for 1 hour and then treated with ultrasound at the indicated power densities and cell counts determined as described for Example 1. In addition, samples of cells were analysed using a Becton Dickinson flow cytometer in order to determine leakage of fluorescein following exposure to ultrasound.

Results 2

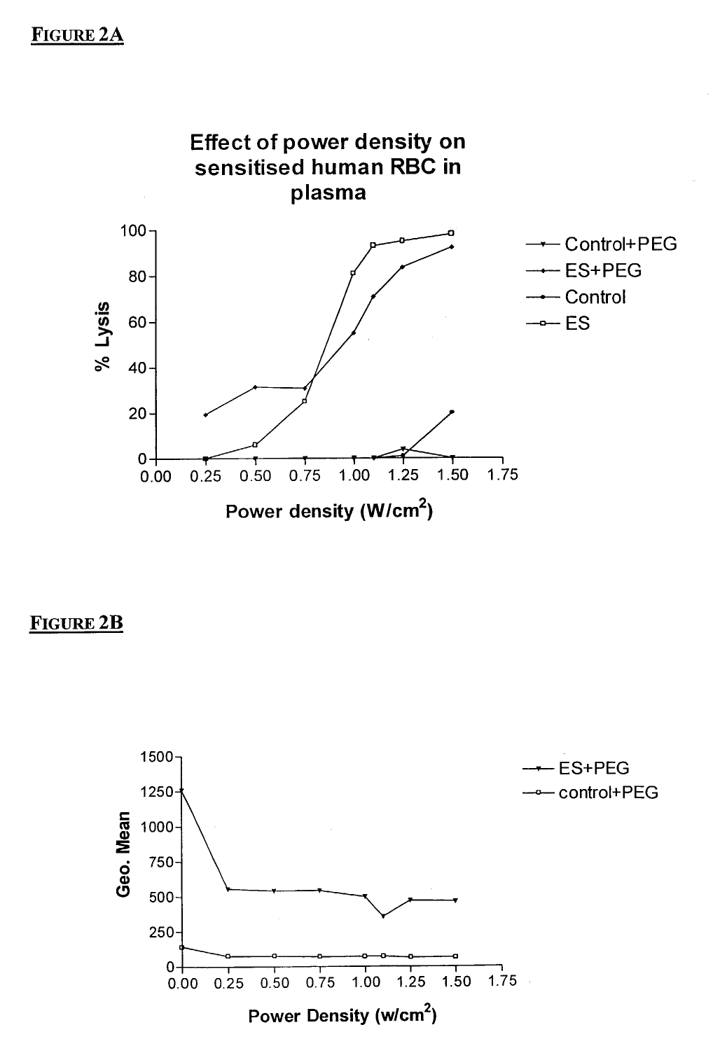

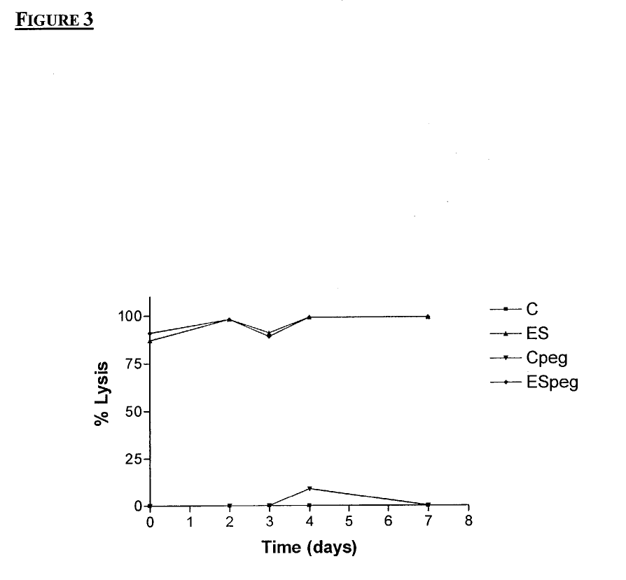

The results are shown in FIG. 2A and they demonstrate that exposing normal and PEG-treated control cell populations in the presence of plasma to ultrasound has little or not effect on those cells at power densities ranging from 0.25-1.5 W/cm². However, exposure of the electro-sensitised normal and PEG-treated cells to ultrasound in the presence of plasma results in increasing cell lysis with increasing power density (FIG. 2A). These results demonstrate that the susceptibility of both electro-sensitised human red blood cells and PEG-treated human red blood cells to ultrasound remains in the presence of autologous plasma. In addition, it was found that ultrasound-mediated release of the loaded fluorescein was achieved following exposure of the electro-sensitised, PEG-treated cells to increasing power densities as demonstrated by a decrease in the geometric mean of fluorescence using flow cytometry (FIG. 2B). Control populations of cells consisted of exposing PEG-treated cells exposed to fluorescein in the absence of an electo-sensitisation event (FIG. 2B). These results demonstrate ultrasound-mediated leakage of a pay-load from the hyper-sensitised, PEG-treated red blood cells in the presence of autologous plasma.

### Example 3

### Stability of Electric Field-Mediated Sensitisation of Normal and PEGTreated Human Red Blood Cells to Ultrasound During Prolonged Storage in PBS/Mg/Glucose

Examples 1 and 2 demonstrated that exposure of both normal and PEG-treated red blood cells to electric field conditions suitable for loading cells coincidentally conferred upon those cells hyper-sensitivity to ultrasound. It was decided to determine whether or not hyper-sensitivity persisted during storage. Cells were harvested and electro-sensitised as described for Example 1. Cells were subsequently re-sealed in PBS/Mg for 30 min. and a proportion were treated with PEG as described by Scott et al. (1997) Proc. Natl. Acad. Sci. (U.S.A.), 94, 7566-7571). Cells were then suspended in PBS/Mg/glucose (supplemented with 1% v/v penicillin/streptomycin solution [50001 U/ml] and 1% v/v gentamicin [10 mg/ml]) to yield concentrations of 14×10⁸ cells/ml. Cells were stored at 4° C. and samples were treated with ultrasound (1.25 W/cm² for 30 seconds using a 3 MHz ultrasound head) as described in Example 1. The degree of cell lysis was determined using a hemocytometer.

Results 3

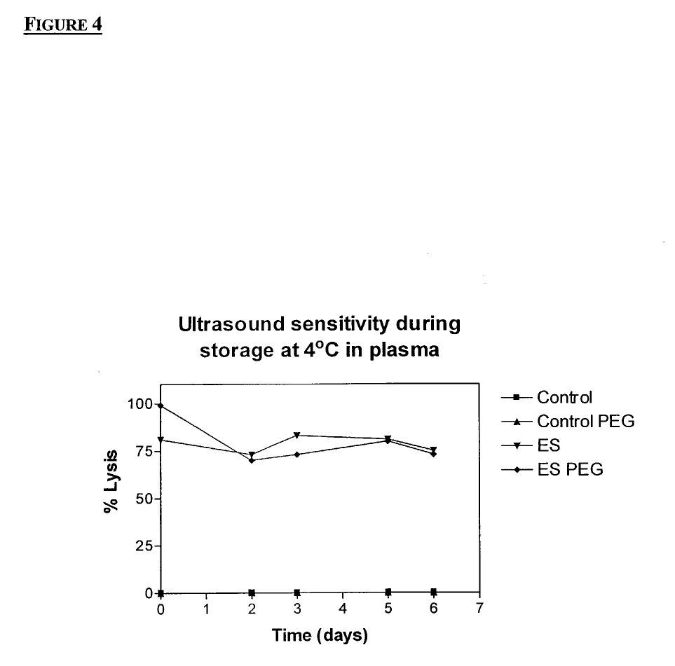

The results are shown in FIG. 3 and they demonstrate that both normal and PEG-treated cells exhibit little or no susceptibility to ultrasound during storage over a 7 day period. The results also demonstrate that the degree of ultrasound sensitivity induced by electro-sensitisation in both red blood cells and PEG-treated red blood cells is retained over this period of time. It was concluded from this experiment that the electro-sensitisation phenomenon exhibited by both the red blood cells and the PEG-treated red blood cells is retained during storage at 4° C. in PBS/Mg/glucose.

### Example 4

### Stability of Electric Field-Mediated Sensitisation of Normal and PEGTreated Human Red Blood Cells to Ultrasound During Storage in Autologous Plasma

It has been shown in Example 3 that cells which had been rendered ultrasound sensitive using electro-sensitisation remained sensitive for prolonged periods of time during storage on PBS/Mg/glucose. It was of interest to determine whether or not this phenomenon could be retained for prolonged periods of time during storage in autologous plasma. To this end both sensitised and non-sensitised normal and PEG-treated cells were prepared as described for Example 3. Samples were re-sealed for 30 min in PBS/Mg and subsequently transferred to PBS/Mg/glucose for 15 min. Cells were then suspended in autologous plasma supplemented with 1% v/v penicillin/streptomycin solution [5000 IU/ml] and 1% v/v gentamicin [10 mg/ml]) to yield concentrations of 13×10⁸ cells/ml. Cells were stored at 4° C. and samples were treated with ultrasound as described in Example 3.

Results 4

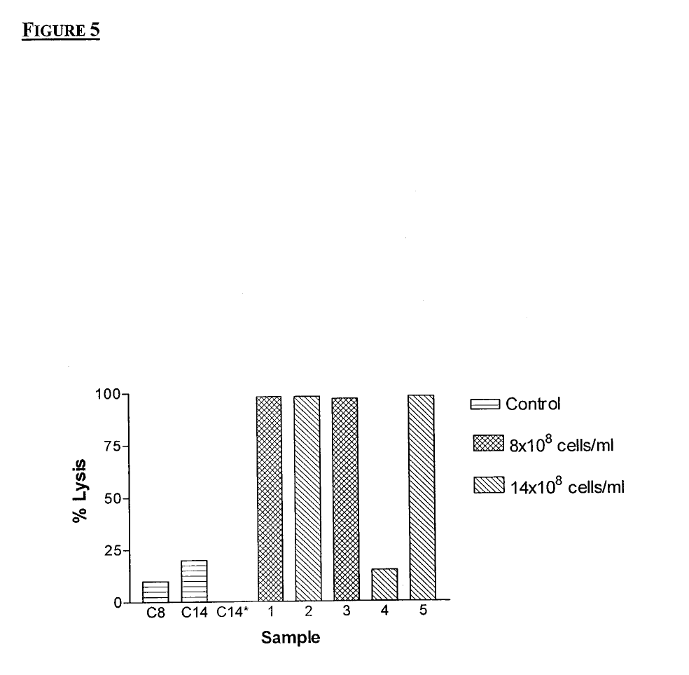

The results obtained are shown in FIG. 4 and they demonstrate that normal and PEG-treated red blood cells stored in autologous plasma for 6 days remained insensitive to ultrasound. However, electro-sensitised normal and PEG-treated cells exhibited hyper-sensitivity to ultrasound over the time period examined. These results demonstrate that the electro-sensitised cells retained their sensitivity even over prolonged periods of time in autologous plasma.

### Example 5

### Effect of Electroporation Conditions on Sensitivity of Human Red Blood Cells to Ultrasound

Since the above results demonstrated that exposure of human red blood cells to short duration (0.02 mseconds) double electric pulses of 1.45 kV (3.625 kV/cm) at a capacitance of 1 uF conferred upon those cells sensitivity to lysis by ultrasound it was decided to determine whether or not alternative electric pulse conditions might yield similarly sensitised cells. The wave form of the above pulses is referred to as and exponential and describes decay of the delivered pulse across the electrodes. Alternatives include either a square wave or a modulated wave decay across the electrodes in the electroporation cuvettes. In order to examine a representative range of electrical conditions it was decided to study a number of parameters associated with exponential wave decay. These included pulse number delivered to the cuvettes, the capacitance of the pulse(s) delivered, the voltage of each pulse and the effect of delivering sequential pulses at varying voltages. Cells were suspended at not greater than 7×10⁸ cells/ml in PBS, exposed to the conditions described in Table 1 and finally suspended in PBS/Mg/glucose for at least 1 hour prior to exposure to ultrasound. Conditions for exposure to ultrasound were those described for Example 1. Cell lysis was determined by counting surviving cells. In addition, it was decided to examine the effects of delivering both square wave and modulated wave pulses to cells and to assess such delivery in terms of susceptibility to ultrasound. In these cases cells were exposed to electric pulses in electroporation cuvettes with a 0.2 cm electrode gap. Cells were again suspended in PBS during delivery of pulses and subsequently exposed to ultrasound following suspension in PBS/Mg/glucose for 1 hour. Cell lysis was determined by counting cells remaining after treatment with ultrasound. In all of the above studies control populations of cells, which had not been exposed to electric pulses, were treated with ultrasound and cell lysis was determined by cell counting.

Results 5

The results obtained in this series of studies are shown in Table 1 below and they and a number of features in terms of inducing ultrasound sensitivity are evident: (i) Ultrasound sensitivity may be induced using pulses delivered as exponential, square or modulated wave forms. (ii) Increasing the pulse number using exponential wave delivery increases red blood cell sensitivity to ultrasound at lower voltage (see Table 1; 0.7 kV, 1 uF single and double exponential pulse). (iii) Increasing the capacitance at lower pulse voltages increases the ultrasound sensitivity (Table 1; 0.6 kV, 1 uF). (iv) Sequential delivery of low, high and finally low voltage using exponential wave form results in ultrasound sensitivity. (v) Ultrasound sensitivity may be induced using pulses with exponential, square and modulated wave forms. These results demonstrate that ultrasound sensitivity may be induced using a relatively wide variety of electrical parameters.

TABLE 1
CONDITIONS SUITABLE FOR SENSITISATION TO ULTRASOUND

| Conditions | U/S sensitivity |
|---|---|
| Exponential wave⁺ | % cell lysis |
| Single pulse | |
| 0.7 kV, 1 uF | 15 |
| 1 kV, 1 uF | 80 |
| 1.45 kV, 1 uF | 100 |
| Double pulse | |
| 0.7 kV, 1 uF | 84 |
| 1 kV, 1 uF | 88 |
| 1.45 kV, 1 uF | 96 |
| Sequential pulsing | |
| 0.3 kV, 10 uF; 1.45 kV, 1 uF: 0.3 kV, 10 uF | 98 |
| Increased capacitance | |
| 0.6 kV, 1 uF | 4 |
| 0.6 kV, 10 uF | 86 |
| Using BioRad RF module* (square wave) | |
| 0.1 kV, 40 kHz, 100 ms burst, 1 s burst interval, 5 bursts | |
| 0% modulation (square wave) | 90 |
| 100% modulation | 91 |

⁺All samples were treated in 0.4 cm cuvettes and electric pulse conditions were delivered using a BioRad Gene Pulsar apparatus. Voltages refer to those delivered by the apparatus.
*The BioRad RF module is designed to deliver either square wave or modulated wave pulses. In all of the above studies pulses were delivered to cells suspended in PBS. Cells were exposed to ultrasound as described for Example 1 using 1.25 W/cm² for 30 s. In all cases control populations of cells at the same concentration were exposed to ultrasound. In those control sample no significant lysis was detected following exposure to ultrasound

### Example 6

### Effect of Cell Concentration on Electric Field Mediated Sensitisation of Human Red Blood Cells to Ultrasound

The purpose of this study was to determine whether increasing the concentration of the electro-sensitised cells resulted in decreased responses to ultrasound. In order to determine whether or not this might be the case samples of cells were harvested as described for Example 1 and cell concentrations were adjusted to 8 and 14×10⁸ cells/ml. Populations are then electrosensitised, placed in PBS/Mg for 30 minutes to re-seal and subsequently suspended in PBS/Mg/glucose for storage at room temperature. Both populations were stored for 1 h and 3 h prior to treatment with ultrasound as described for Examples above (3 MHz, 1.5 W/cm², 5 min.) and cell counts were determined 30 min. after treatment. Control samples were treated in a similar manner although the electroporation event was omitted.

Results 6

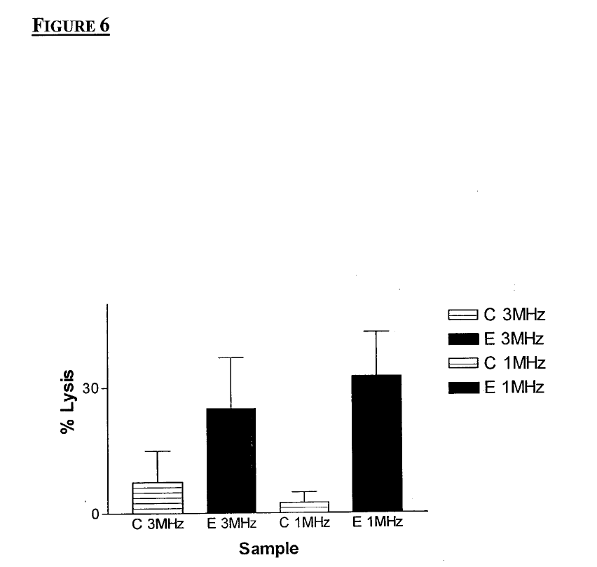

The results are shown in FIG. 5 and they indicate little or no effect in control samples (C8 and C14) for both cell concentrations. When cells were stored for 1 h and treated with ultrasound, lysis in both populations was 98-99% (FIG. 5; Samples 1 and 2). However when samples were stored for 3 h and subsequently treated with ultrasound, lysis of the population containing 8×10⁸ cells/ml was 99% whereas that of the population containing 14×10⁸ cells/ml was only 15% (FIG. 5; Samples 3 and 4, respectively). These results suggested that cells at the higher concentration had the ability to recover from electro-sensitisation during storage. Since a higher concentration of cells had been employed in the electro-sensitisation process it seemed reasonable to presume that electric field-mediated effects on individual cells within the overall population would be reduced. This may have resulted in the ability of those individual cells to recover from sensitisation. In order to test this hypothesis, cells were harvested and electrosensitised in aliquots containing 8×10⁸ cells/ml. Cells were allowed to recover in PBS/Mg at that concentration for 30 min. and subsequently pooled to yield 14×10⁸ cells/ml in PBS/Mg/glucose for storage (RT). Cells were treated with ultrasound following 3 h storage and cell counts were determined 30 min. later. Control samples (C14*) were treated in a similar manner without delivery of the electro-sensitisation pulses. The results are shown in FIG. 5 (Sample 5) and it would found that 99% of the cells lysed following treatment. These results confirmed out hypothesis and demonstrated that in order for ultrasound-sensitivity to persist at higher cell concentrations it would be necessary to perform the electro-sensitisation procedure at lower cell concentrations. It would alternatively suggest that electro-sensitisation of human red blood cells at higher cell concentrations would require adjustment of the electrical parameters delivered to those cells in order to sustain ultrasound sensitivity during storage.

### Example 7

### Effects of Ultrasound on Electro-Sensitised Human Red Blood Cells Placed in a Soft Tissue Phantom

The results above demonstrate that electro-sensitisation of human red blood cells to ultrasound may be achieved in such a manner that those cells may be selectively lysed using ultrasound parameters which have little or no effect on normal human red blood cells in vitro. The purpose of this study was to demonstrate the selective effect in vivo. To this end a soft tissue Doppler phantom was employed (supplied by Dansk Fantom Service, Denmark). The phantom consists of a matrix which transmits sound at a mean velocity of 1503 ms⁻¹ at 3 MHz, attenuates sound at 0.54 dBcm⁻¹* MHz and has a density of 1040 Kgm⁻³. The system contains tubing with an inner diameter of 1.6 mm and an outer diameter of 3 mm which travels through the matrix at a 25° angle to the scanning surface. Samples of cells (7-8×10⁸) were injected into the tubing such that each sample treated was at an average depth of 1 cm from the scanning surface. Cells used in the system were harvested, electro-sensitised as described above in Example 1, re-sealed in PBS/Mg for 30 min. and subsequently injected into the phantom. Control samples were treated in a similar manner except that electro-sensitisation was not carried out. Samples were treated with the 3 MHz and 1 MHz ultrasound heads for 5 min.

Results 7

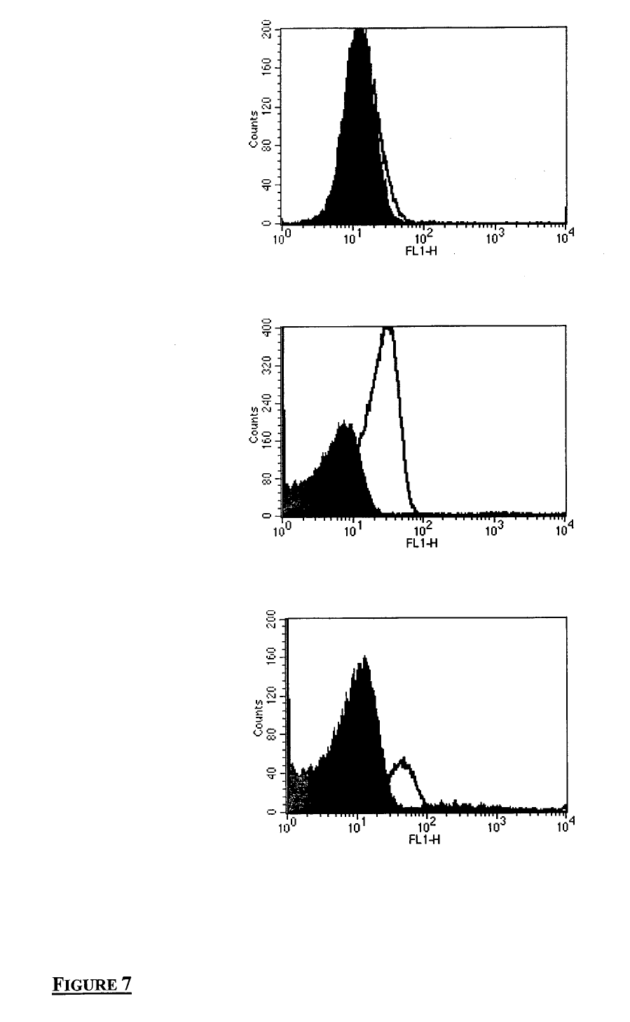

The results obtained are shown in FIG. 6 and they demonstrate that at 3 MHz treatment resulted in an average of 7% lysis whereas treatment of the electro-sensitised samples resulted in 24% lysis. At 1 MHz treatment resulted in 3% lysis in control samples and 34% lysis in samples which had been electro-sensitised. These results demonstrated that preferential lysis of electro-sensitised human red blood cells may be achieved in a soft tissue vascular mimicking system. The results suggest that ultrasound could be exploited as a non-invasive means of releasing pay-load from a delivery vehicle which has been electro-sensitised.

### Example 8

### Electrosensitisation

To demonstrate that electrosensitisation of red blood cells occurs in the absence of electroporation under consditions of insufficient electric field energy to achieve electroporation, an experiment was designed in which a FITC-labelled polyclonal antiserum was loaded into red blood cells by electroporation. Cell lysis in response to ultrasound was assessed on a haemocytometer as in Example 1. The field strength was then reduced was then reduced to a point at which no antibody loading was observed, and cell lysis in resoponse to ultrasound was again assessed.

In a first experiment, 4.5×10⁷ RBCs were incubated with 0.25 mg/ml antibody, cooled to 4° C. and electrosensitised by pulsing at 0.3KV 10 μF, 1.45KV 1 μF and 0.3KV 10 μF in phosphate-buffered sucrose (PBSucrose).

In a second experiment, 3.5×10⁷ RBCs were incubated with 0.25 mg/ml antibody, cooled to 4° C. and electrosensitised by pulsing at 0.3KV 10 μF, 1.45KV 1 μF and 0.3KV 10 μF in PBS.

In a third experiment, 5×10⁷ RBCs were incubated with 0.5 mg/ml antibody, cooled to 4° C. and electrosensitised by pulsing twice at 1.45KV 1 μF in PBS.

Results 8

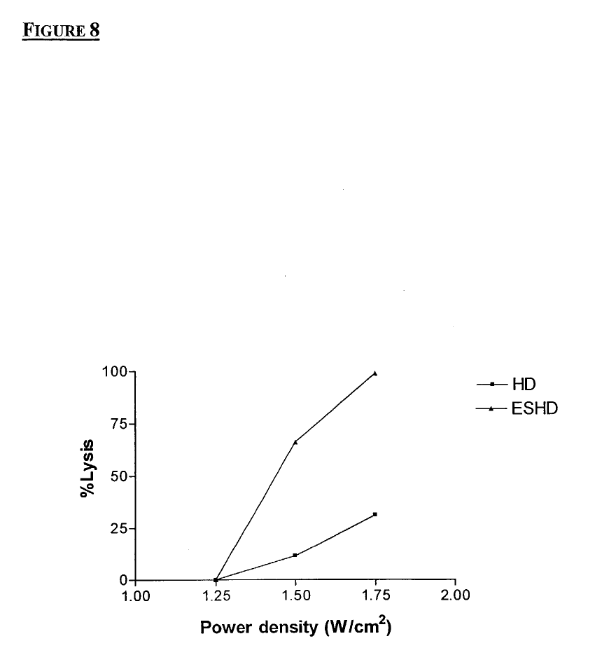

FIG. 7 shows the results of this experiment. The first and second experiments, pulsing at 0.3KV 10 μF, 1.45KV 1 μF and 0.3KV 10 μF in PBS or PBSucrose, showed antibody loading at ratios of 3.98 and 4.04 respectively as determined by FACS (FIG. 7, middle and lower panels). The cells are also 100% sensitive to ultrasound as determined by haemocytometer counting, according to Example 1.

The third experiment, pulsing twice at 1.45KV 1 μF in PBS, showed no antibody loading when analysed by FACS (FIG. 7, upper panel). However, the cells remained 100% sensitive to ultrasound as determined by haemocytometer counting.

### Example 9

### Electro-Sensitisation of Human Red Blood Cells Treated with a Hypotonic Dialysis Loading Protocol

The objective of these experiment was to determine whether or not it would be possible to electro-sensitise human red blood cells which may be loaded using alternative loading modalities. It was decided to employ a protocol designed to achieve loading using hypotonic dialysis as described by Eichler et al., 1986, Clin. Pharmacol. Therap. 40: 300-303. Essentially, washed red blood cells were suspended in 1 ml of PBS (150 mM NaCl, 5 mM K₂HPO₄/KH₂PO₄; pH 7.4) to obtain a hematocrit of 60%. This suspension was placed in dialysis tubing (molecular weight cutoff 12-14,000; Spectra-Por) and swelling of cells was obtained by dialysis against 100 ml of 5 mM K₂HPO₄/KH₂PO₄, pH 7.4 for 90 minutes at 4° C. Resealing was achieved by subsequent dialysis for 15 minutes at 37° C. against 100 ml of PBS containing 10 mM glucose. Cells were then washed using centrifugation. In cases where electro-sensitisation was performed the method described in Example 1 was employed and cells were subsequently placed in PBS/Mg (Example 1) for 30 min at room temperature. All cells were subsequently stored in PBS/Mg/glucose (Example 1) prior to treatment with ultrasound.

Results 9

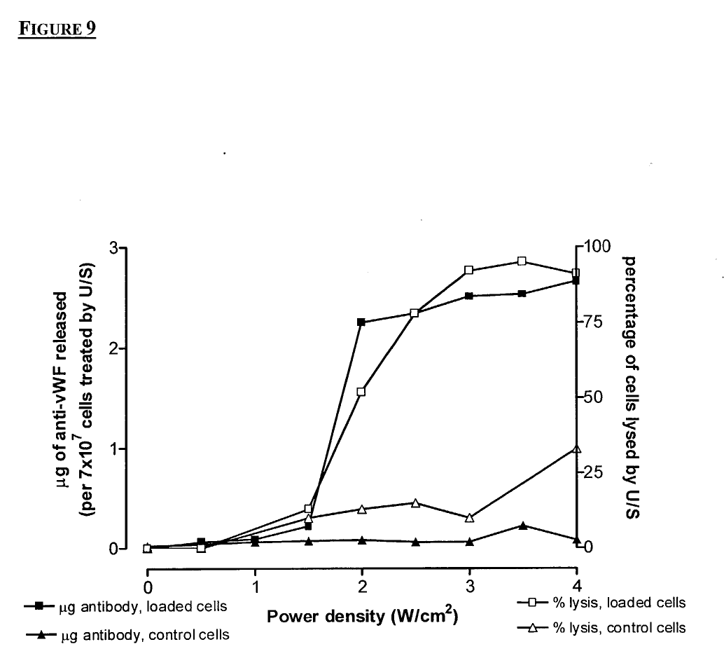

Following hypotonic dialysis and exchange into PBS/Mg/glucose, cells were stored at room temperature for 1 hour. Both cells treated with hypotonic dialysis (HD) and electro-sensitised cells which had been treated with hypotonic dialysis (ESHD) were then exposed to ultrasound at power densities of 1.25, 1.5 and 1.75 W/cm² and at a frequency of 3 MHz as described for Example 1. No significant lysis was observed in electro-sensitised samples above that exhibited by control cells which were treated with hypotonic dialysis alone. Indeed at 1.25 W/cm² no lysis was observed in either sample and this contrasts significantly with results presented in FIG. 1, Example 1 where electro-sensitised normal cells exhibited almost 100% lysis at that power density. When both the HD and ESHD cells were stored overnight at 4° C. and subsequently exposed to ultrasound, no significant lysis was detected. It was subsequently decided to electro-sensitise the HD cells which had rested overnight. Following electro-sensitisation, cells were placed in PBS/Mg (Example 1) for 30 min. and subsequently placed in PBS/Mg/glucose for 1 hour. Cells (at a concentration of 5×10⁸ cells/ml were exposed to ultrasound at 1.25, 1.5 and 1.75 W/cm² as described above and the degree of lysis was determined. The results are shown in FIG. 8 and they demonstrate that the electro-sensitised samples exhibited preferential lysis following exposure to ultrasound at 1.5 and 1.75 W/cm² . The results from these experiments demonstrate that it is possible to render human red blood cells sensitive to ultrasound following treatment with alternative procedures designed to achieve loading of those cells. However, cell treated with the hypotonic dialysis method must be allowed to rest for a period of time prior to electro-sensitisation.

### Example 10

### Release of Payload from Loaded and Sensitised Vehicle in a Tissue Mimicking System (TMM)

Since the previous examples demonstrated that human erythrocytes could be sensitised to low intensity ultrasound, it was decided to show that a payload entity loaded into those sensitised erythrocytes could be released following exposure to low intensity ultrasound. In these experiments the target is placed at a distance of 1.3 cm from the emitting surface of the ultrasound head and the intervening space is filled with a tissue mimicking material (TMM) which attenuates ultrasound in the same manner as a soft tissue. The TMM chosen for this work is described in Madsen et al. (1998, Ultrasound Med. & Biol., 24, 535-542) and following preparation, care is taken to ensure that the material has a density of 1.03 g/ml.

In previous examples sensitised cells were employed as the target. In Example 9 above it is shown that cells which have been processed using technologies designed to load erythrocytes can be rendered sensitive by exposure to electric pulses. In order to demonstrate ultrasound-mediated payload release from the vehicle it was decided to employ a loaded vehicle as the target.

It was decided to employ a loading modality which incorporated a pre-sensitising step prior to loading by hypotonic dialysis and subsequent exposure of those cells to an electrosensitising step. The pre-sensitising step is an optional step which increases the efficiency of a subsequent loading step, and is described in detail in our co-pending British Patent Application GB0002856.3. Thus, in this and the following examples in which RBCs are loaded with payload, a pre-sensitising step is conducted to ensure optimal loading of payload into the RBCs. After loading of the agent, the cell is subjected to a second electrosensitising step, which sensitises the cell to ultrasound. As noted above, the pre-sensitising step is optional, and identical or similar results are obtained when this step is omitted in the following examples.

Loading Protocol

This section describes protocols for the loading and sensitisation of red blood cells by a combination of electrosensitisation (ES) followed by hypoosmotic dialysis loading (HD) overnight rest and further treatment of the cells by electrosensitisation. This combination is abbreviated as ES+HD+ES.

10 ml of peripheral venous blood is collected by venipuncture, into lithium heparin anticoagulant containing tubes, and mixed gently. The whole blood is then poured into a polypropylene tube and centrifuged at 300 g for 15 min at room temperature. The plasma and white blood cells (buffy coat) are removed.

1 × phosphate buffered saline (PBS, made from Oxoid tablets code BR14a pH7.3) is added and the cells centrifuged at 700 g for 5 min. The supernatant is removed and the pellet of remaining cells resuspended in ice cold 1×PBS. The spin/wash procedure is then repeated once, and cells are suspended in ice-cold PBS at 6×10⁸ cells/ml.

Cells are then electrosensitised by dispensing 800 μl of the RBC into sterile electroporation cuvettes, and placed on ice. To electrosensitise the cells, they are exposed to an electric field at 3.625 kV/cm, 1 μF (2 pulses), in the absence of payload. The RBCs are then removed, and pooled in polypropylene tubes.

Cells are centrifuged once at 700 g for 5 min at room temperature (RT). The cells may be diluted in PBS/MgCl₂ (4 mM). Cells are then resuspended in PBS/MgCl₂, and centrifuged at 700 g for 5 min, twice. Finally, cells are resuspended in PBS/MgCl₂, at approximately 7×10⁸ c/ml, and rested for 30 min at room temperature.

Cells are then loaded with payload by hypo-osmotic dialysis, according to a protocol adapted from Eichler et al., (1986) Clin. Pharmacol. Ther. 40:300-303. Essentially the protocol is as follows:

1 Reagents and Buffers:

Stock potassium phosphate buffer:

5 mM K₂HPO₄3H₂O(FW 228.2 g)=>1.141 g/L

5 mM KH₂PO₄(MW136.1 g)=>0.68 g/L

Stored at 4° C.

Mix as follows:

For a pH7.4K₂H/KH₂ phosphate buffer=>approx. 6.1:3.9 parts

Mix the 2 stock solutions as and when required

Buffer #1 (isoosmotic PBS):

pH7.4K₂H/KH₂ phosphate buffer

150 mM NaCl=>8.76 g/L

Check and adjust pH (1 M NaOH)

Buffer #2 (dialysis buffer):

pH7.4K₂H/KH₂ phosphate buffer

Check and adjust pH (1 M NaOH)

Buffer #3 (resealing buffer)

pH7.4K₂H/KH₂ phosphate buffer

150 mM NaCl=>8.76 g/L

10 mM glucose =>1.8 g/L

Check and adjust pH (IM NaOH)

SpectraPor Dialysis Tubing Preparation:

1 The 12-14 kDa MW cut off tubing, 0.32 ml/cm, is used.

2 Preparation: heat at 80° C./30min in 1 mM EDTA/2% sodium bicarbonate (Sigma). Rinse well, inside and outside, with ddH₂O.

3 Wash inside and outside with Buffer #1

4 Store submerged in a small amount of Buffer #1 if not used immediately.

RBC Preparation:

1 Electrosensitised, rested RBC are washed in PBS twice at 700 g for 5 min.

2 For the final wash, cells are washed in buffer #1

3 The cells are manipulated as a suspension of packed cells following removal of final wash supernatants after centrifugation.

Cell Volume in Tubing:

1 Protocol recommends 60% haematocrit (HCT). The suspension of packed cells is approximately 75% HCT and is diluted accordingly.

2 Mix cells with the payload and buffer #1, to give required final payload concentration and volume.

2 Dialysis:

1 The tubing is clipped to ensure that the surface area remains constant for the volume of cells.

2 Dialyse RBC (packed cell volume in buffer #1) against buffer #2 for 90 min at 4° C.

3 Place membranes in 100-200 ml buffer #2, (ensure that the membrane is immersed) in glass beaker with magnetic flea.

4 Place this beaker within another beaker, which contains ice, on the magnetic stirrer, cover with silver foil.

6 Warm up an aliquot of buffer #3 to 37° C.

7 Remove dialysis buffer, replace with the warm resealing buffer #3.

8 Place beaker with dialysis tubing and buffer #3 into a larger beaker anchored by water at 37° C., cover and leave for 15 min.

9 Harvest cells into 12 ml polypropylene tubes.

10 Wash ×3 in ice cold resealing buffer #3 at 300 g, 10 min 4° C.

11 Wash ×1 in PBS/Mg/glucose and spin at 700 g, 5 min 4° C.

12 Count cells and resuspend at 7×10⁸ c/ml, in PBS/Mg/glucose.

13 Store at 4° C. overnight.

In the present example, dialysis is performed in the presence of 1.5 mg of antibody per ml of cells. Cells are suspended at 7×10⁸ cells/ml.

When cells have been loaded they are then sensitised by exposure to electric pulses as follows:

3 Electrosensitisation

1 Following overnight storage, wash RBC once in PBS 700 g, 5 min 4° C.

2 Count cells and resuspend at 6×10⁸c/ml, in ice cold PBS.

3 Dispense 800 μl of the RBC into sterile electroporation cuvettes (0.4 cm gap).

4 Place on ice.

5 To electrosensitise: double pulse at 3.625 kV/cm, 1 μF.

6 Harvest the RBC, pool in a polypropylene tube.

7 Centrifuge once at 700 g for 5 min room temperature (RT). The cells may be diluted in PBS/MgCl₂(4 mM).

8 Resuspend in PBS/MgCl₂, centrifuge at 700 g for 5 min.

9 Repeat step 6

10 Resuspend in PBS/MgCl₂, at approximately 7×10⁸c/ml.

11 Rest the cells for 30 min at RT.

12 Centrifuge once at 700 g for 5 min room temperature (RT). The cells may be diluted in PBS/MgCl₂/glucose.

13 Resuspend the cells in PBS/MgCl₂/glucose, centrifuge at 700 g for 5 min.

14 Repeat step 13.

15 Resuspend cells in PBS/MgCl₂/glucose at 7×10⁸ c/ml.

16 Rest the cells in PBS/MgCl₂/glucose for 60 min.

I. Ultrasound-mediated release of antibody from vehicle

Antibody-loaded sensitised cells are then exposed to ultrasound at a distance of 1.3 cm from the emitting surface of the ultrasound head. The intervening space is filled with the TMM as described above and 0.1 ml aliquots of 7×10⁸ cells/ml are exposed to ultrasound. In these studies a sheep anti-human von Willebrand factor antibody is employed as the payload in these studies. The amount of antibody in cells and released following treatment with ultrasound is quantified with an ELISA system, using standard protocols (as disclosed in for example, Harlow and Lane, Antibodies: a Laboratory Manual, (1988) Cold Spring Harbor, and Maniatis, T., Fritsch, E. F. and Sambrook, J. (1991), Molecular Cloning: A Laboratory Manual. Cold Spring Harbor, N. Y., Cold Spring Harbor Laboratory Press).

I Results

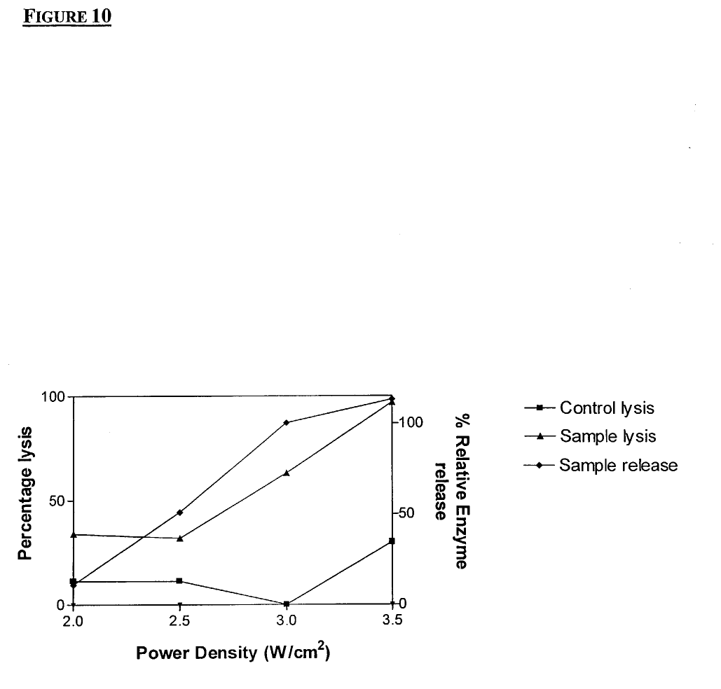

In the loading and sensitisation protocol, cells are loaded at a concentration of 1.1 mg of antibody per ml of packed cell volume (PCV). 0.1 ml aliquots of cells at 7×10⁸ cells/ml are exposed to ultrasound at intensities shown in FIG. 9 and samples re analysed for cell lysis by direct counting. In addition, the amount of antibody released following treatment with ultrasound is determined by ELISA analysis of cell supernatants harvested following centrifugation. The results obtained are shown in FIG. 9 and they demonstrate that cells were preferentially lysed at ultrasound power densities greater than 2 W/cm². Control cells exhibit little or no effect when treated with ultrasound at these power densities. At and above 2 W/cm² antibody payload is detected in supernatants harvested following ultrasound treatment. In addition, when the total amount of antibody released from the cells using ultrasound is compared with that released following hypotonic lysis in 0.01% (v/v) Triton X100 it is found that 77% of the total antibody is released in the former. The remainder could be found in debris that is recovered by centrifugation following ultrasound treatment.

The results demonstrate that ultrasound-mediated release of payload can be achieved using relatively low intensity ultrasound and using conditions which have little or no effect on normal erythrocytes. The results also demonstrate ultrasound-mediated release of payload at a depth of 1.3 cm and thereby demonstrating one of the major advantages associated with the use of ultrasound as the releasing stimulus of penetration to depth in tissues. Since all of the antibody incorporated into the ultrasound treatment experiments can be recovered as shown using an ELISA based on payload functionality, this suggests that the ultrasound has no detrimental effect on that functionality

II. Ultrasound-Mediated Release of Enzyme (β-Galactosidase) from Vehicle

Cells are harvested, primed by exposure to electric pulses and loaded with μ-galactosidase (from Escherichia coli, Sigma) as described above for antibody loading. Cells are subsequently exposed to sensitising electric pulsing and exposed to ultrasound at a concentration of 7×10⁸ cells/ml in the TMM system as described above for the antibody-loaded vehicle. Lysates obtained following exposure of the loaded and sensitised vehicle to ultrasound are assayed for β-galactosidase activity at 3720 C. using the colorimetric substrate p-nitrophenyl-β-D-galactoside (5 mM in 50 mM phosphate buffer, pH 7.0). The concentration of p-nitrophenol is determined spectrophotometrically at 450 nm and activity is expressed as μmoles of p-nitrophenol produced per minute per ml of sample. Release of enzyme in samples harvested following treatment with ultrasound is expressed as a percentage relative to the amount of enzyme contained in the cells prior to treatment. The latter is determined by measuring the amount of enzyme released from the cells following lysis by freeze-thaw in 5 mM phosphate buffer, pH 7.2.

II. Results

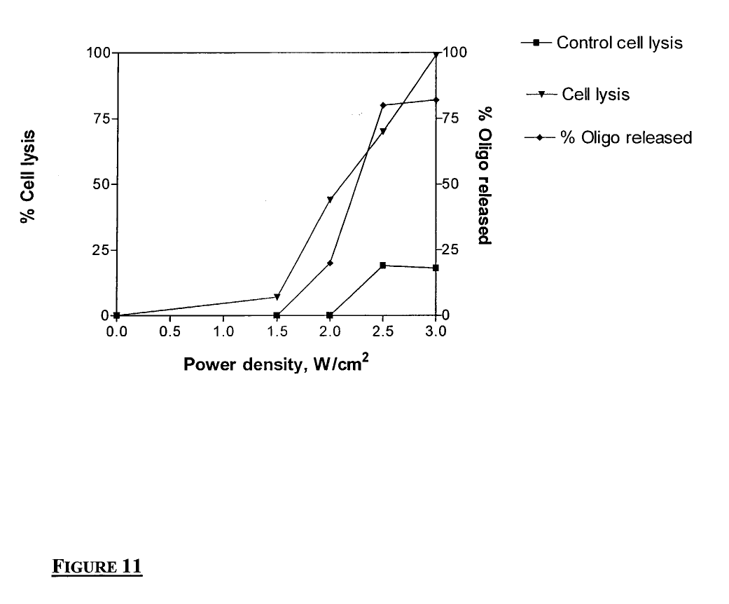

In these experiments loaded cells contain approximately 1 mg of enzyme per ml of packed cell volume. The results obtained following treatment of these preparations with ultrasound are shown in FIG. 10. Samples are treated at the indicated power densities as shown and samples are analysed for cell lysis by cell counting. Lysis increases with increasing power density up to a maximum at about 3 W/cm². Exposure of control normal cells to similar ultrasound conditions has little or no effect on cell lysis and this is confirmed by the absence of hemoglobin in supernatents following removal of cells by centrifugation. When supernatants are harvested by centrifugation, following exposure of the sensitised and loaded cells to ultrasound and analysed for enzyme content, it is found that increasing amounts of enzyme are released with increasing power density up to a maximum at 3 W/cm².

The results demonstrate that enzyme is released from the vehicle following exposure to low intensity ultrasound. Ultrasound-mediated release of the payload is achieved at 1.3 cm from the emitting ultrasound head, indicating that the use of ultrasound for this purpose offers the advantage of penetration to depth in tissues. In addition, since 100% recovery of the enzyme released is achieved (between 2.5-3 W/cm² ), the ultrasound stimulus resulting in release of enzyme has no detrimental effect on the functionality of the released payload.

III. Ultrasound-Mediated Release of Oligonucleotide from Vehicle

Cells are harvested and primed by exposure to electric pulses as described above. Cells are then loaded using the hypoosmotic dialysis procedure described above for antibody loading and a 300 μg quantity of oligonucleotide (tamara labelled random 30-mer supplied by Oswel, UK) is mixed with 250 μl of packed cells. Samples are then subjected to electrosensitisation electric pulses and subsequently suspended in PBS/MgCl₂/glucose at a concentration of 7×10⁸ cells/ml. Samples are exposed to ultrasound using the TMM system described above for antibody and enzyme release and the amount of oligonucleotide released is determined using a spectrofluorimeter (Shimadzu) with excitation set at 540 nm and emission set at 590 nm. A standard curve is constructed for quantitative determinations and extraction efficiencies are taken into account.

III. Results

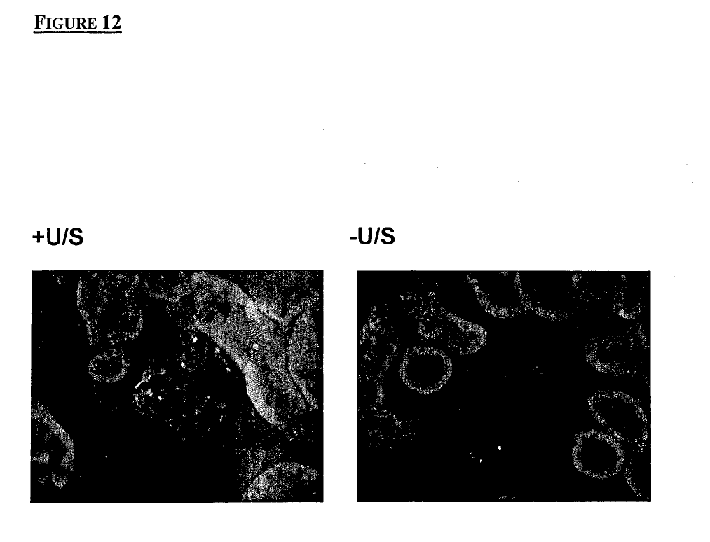

In these experiments the maximum amount of oligonucleotide loaded is approximately 300 μg of oligonucleotide per ml of packed cell volume. The results obtained following treatment of these loaded preparations with ultrasound are shown in FIG. 11. As with the above two examples, cell lysis of the sensitised and loaded preparation occurs between 2 and 3 W/cm². Under these ultrasound conditions there is little or no effect on control erythroyctes. In addition, oligonucleotide begins to appear in harvested supernatants between 2 and 3 W/Cm² demonstrating ultrasound-mediated release of oligonucleotide payload from the vehicle. These results also demonstrate release of the payload from the vehicle when the target cells are at a distance of 1.3 cm from the emitting surface of the ultrasound head and this again indicates the advantage of penetration to depth in tissues associated with the use of ultrasound as a release stimulus.

### Example 11

### Ultrasound-Mediated Release of Antibody Payload in a Perfused Rat Kidney System

Although it is demonstrated in the above examples that a variety of payloads can be released in a TMM system it is also of interest to demonstrate release of a functional payload in a tissue. To this end human erythrocytes are loaded with FITC-labelled anti-von Willebrand factor antibody and sensitised as described in Example 10 above. These cells are then administered to PBS-perfused rat kidneys and treatment with ultrasound is carried out according to the following protocol:

Perfuse the rat through the heart with PBS/EDTA until the kidney is clear of blood

Remove the dorsal aorta from the heart and insert a gavage needle into the vessel. Tie the needle to the dorsal aorta using suture.

Close the dorsal aorta and posterior vena cava just after the junction leading to the kidney.

Close the left adrenal artery and vein and both anterior mesenteric and coeliac arteries

Close the ureter and the left iliolumbar artery and vein.

Create an exit point by inserting a gavage needle into the vena cava just before the liver. Tie the needle using suture.

Flush with 10 ml PBS/4 mM Mg/10 mM glucose and check for any leakage.

Block the exit point by inserting 2 ml syringe into the gavage needle to establish positive pressure.

Load 1 ml of 7×10⁸ cells /ml through the dorsal aorta into the kidney.

Treat with U/S using 1 MHz probe.

Incubate the treated kidney for one hour

Remove the 2 ml syringe and flush through with 2 ml PBS/Mg/glucose

Collect the flush through for cell counting and ELISA

Flush with 50 ml of PBS/EDTA

Flush with 20 ml of 4% neutral buffered formalin (NBF)

Remove the U/S-treated kidney and cut it into two half's and fix in NBF

Prepare tissue sections (12 μm)and stain using Vectastain ABC kit (Vecta Labs) as outlined in the manufacturer's instructions.

Results 11

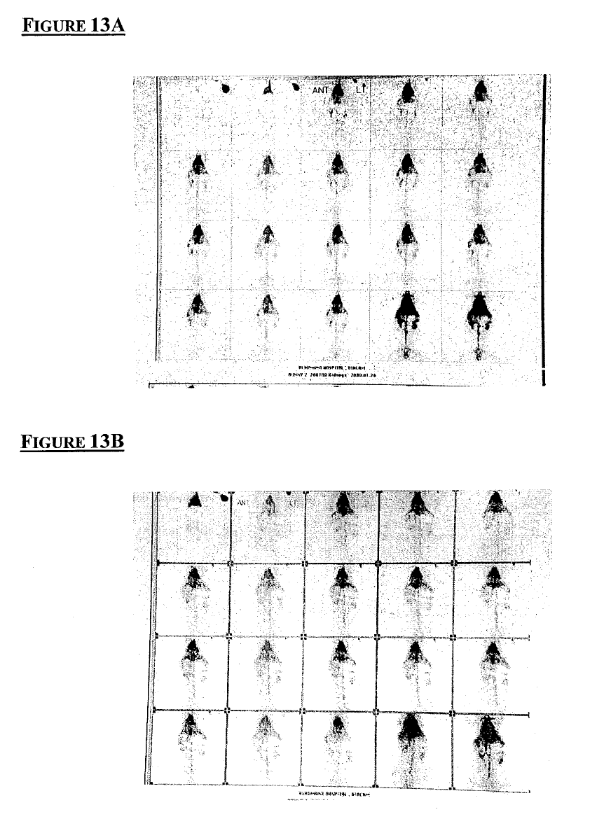

As shown in FIG. 12, kidney endothelial cells in glomeruli are labeled with the FITC conjugated anti-vWF antibody after ultrasound treatment to release the antibody (+U/S). In the absence of ultrasound treatment, no staining is observed ([−U/S]). The results demonstrate that low intensity ultrasound may be used to effect release of functional antibody from the loaded and sensitised vehicle in a perfused tissue system.

### Example 12

### Circulation of Normal, Electrosensitised, Glutaraldehyde-Treated and Pegylated Normal Rabbit Erythrocytes in Vivo

Since it is widely known that damaged erythrocytes are rapidly removed from circulation by macrophages of the reticulo-endothelial system (DeLoach and Barton, 1981, Am. J. Vet. Res. 42, 1971-1974), it is of interest to determine whether or not electrosensitised erythrocytes remain in circulation. As mentioned above it has also been demonstrated that PEGylation has the ability to protect foreign entities from recognition by reticulo-endothelial system and to this end it was decided to PEGylate normal erythrocytes and demonstrate that the modification prevented recognition by that system. In order to examine the above, it was decided to examine circulation of normal, electrosensitised, PEGylated and glutaraldehyde-treated rabbit erythrocytes in vivo. The latter treatment is chosen as a positive control for sequestration by the reticulo-endothelial system since it has been shown to promote targeting by the RES and consequential rapid uptake by the liver/spleen. In these studies circulation of the labelled vehicle is monitored by firstly labelling the cells with ⁹⁹Tc and subsequently monitoring the fate of that label using a gamma camera and acquiring full body scans of the recipient animal.

To the above ends peripheral rabbit venous blood is obtained by venipuncture, and added to tubes containing lithium heparin anti-coagulant. The donor rabbits are male and female New Zealand white rabbits which weighed between 3 kg and 4 kg. Whole blood is centrifuged at 300 g for 15 minutes at room temperature. Plasma and white cells are removed and the cell pellet resuspended to 10 ml in cold PBS. Cells are washed twice at 800×g for 3 minutes and the pellets resuspended with cold PBS. Erythrocytes are diluted in PBS and counted as described above. Peripheral venous blood collected as described previously is aliquoted into Eppendorf tubes and centrifuged at 800 g for 3 minutes at room temperature. Plasma is harvested and stored in Eppendorf tubes at room temperature until required.

In cases where cells are electrosensitised, RBC are resuspended at between 7-8×10⁸ cells/ml in cold PBS. Seven hundred μl of the cell suspension and 100 μl of cold PBS are added to sterile electroporation cuvettes. Cuvettes are mixed and pulsed twice at 1.45 kV and 1 μF with a BioRad electroporator. Cells are harvested into Eppendorf tubes, centrifuged once then washed twice with 1 ml of cold PBS at 800×g for 3 minutes. The final pellet is resuspended with 760 μl of cold PBS/ 4 mM MgCl₂. Cells are left on ice for 30 minutes. Next the cells are washed in a buffer (145 mM NaCl, 2.4 mM KCl, 7.6 mM Na₂HPO₄2H₂O,2.4 mM NaH₂PO₄2H₂O,4 mM MgCl₂, 10 mM glucose). Ultrasound sensitivity following electrosensitisation is verified by exposing the cells to 3 W/cm² for 35 sec. on the TMM system and using the 1 MHz ultrasound head.

In cases where cells are PEGylated the procedure is that described in Scott et al PNAS 94:7566 1997. Cells are washed at 800×g for 3 minutes, resuspended in cold PBS, pooled and counted. Counts are typically between 7-8×10⁸ cells/ml. In order to determine whether PEGylation is successful 1 μl of the appropriate agglutinating IgM antibody (Lorne Laboratories Ltd) is added to 10 μl of a 1:2 dilution of PEGylated RBC and to 10 μl of a 1:2 dilution of non-PEGylated RBC. The glass slides are rocked gently, agglutination is observed in non-PEGylated samples but not in the PEGylated samples.

Cells are glutaraldehyde treated as follows: 1 ml of packed cells are technetium labelled as described above. 0.5 ml of glutaraldehyde at 2.5% v/v was then added to the labelled cells, and the preparation allowed to stand for 5 minutes at room temperature. Cells are then washed twice with PBS.

In all cases, preparations of cells are ⁹⁹Tc labelled for monitoring purposes in vivo. Essentially one ml of the prepared RBC (between 7-8×10⁸c/ml) and 0.5 ml of autologous plasma are mixed and then injected into the technetium kit as per the kit protocol (Mallinckrodt UK Ltd, Bichester, UK). Following labelling, the contents of the kit are harvested and the cells washed 3 times in PBS at 800×g for 3 minutes. Supernatants are retained to check the levels of radioactivity. The final cell pellets are resuspended in 1 ml of PBS and the cells counted. The cells are then injected into the right ear of the rabbits (male or female New Zealand white rabbits, which weighed between 3 and 4 kg). Following injection, the fate of circulating RBC is monitored with a gamma camera and whole body images are acquired at times ranging from time zero to 20 minutes.

Results 12

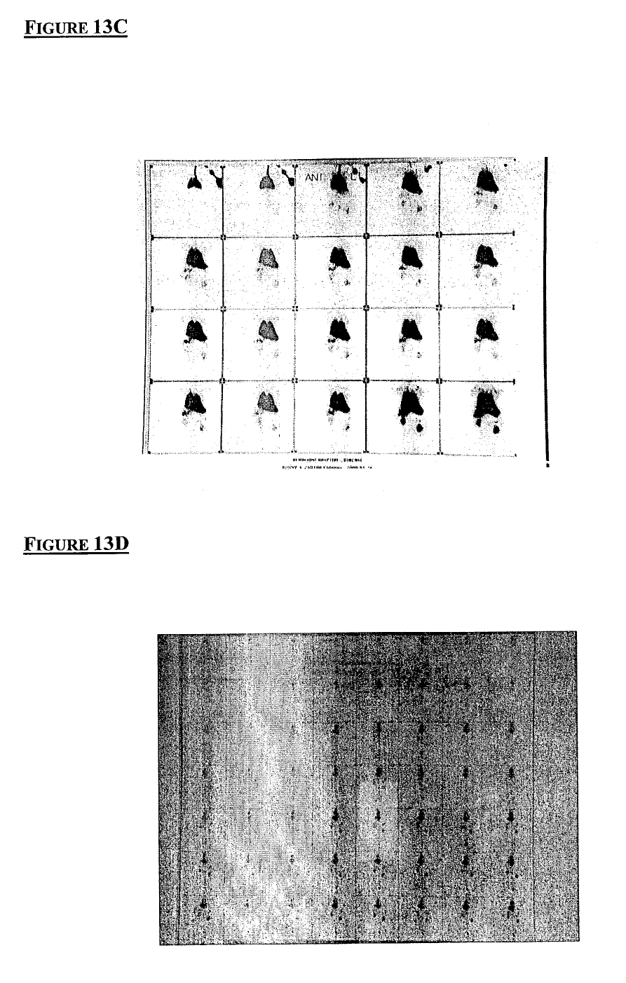

Results are presented as four panels in FIGS. 13 A,B,C and D representing whole body scans of rabbits injected with electrosensitised, normal, gluraraldehyde treated normal and PEGylated normal rabbit erythrocytes, respectively. In panels A to C, whole body scans are captured at the times indicated in FIG. 13 over a period of time zero to 20 minutes. In FIG. 13, panel B the distribution of labelled normal erythrocytes is seen over this time period and represents a normal distribution. Liver, kidneys and ventral line vasculature to fermorals are clearly imaged and no preferential accumulation in the liver-spleen is seen in the images. The distribution of electrosensitised rabbit cells (FIG. 13, Panel A) over the scanning time period is similar to that exhibited by normal cells and from this one can conclude that the half life of the sensitised cells mimics that of normal cells. No preferential accumulation in the liver or spleen over that exhibited by the normal cells is apparent. As mentioned above, glutaraldehyde treatment of erythroyctes promotes recognition by the reticulo-endothelial system with the consequence of rapid sequestration by the liver and spleen. FIG. 13, panel C clearly shows dramatic premature accumulation of the radioactively labelled glutaraldehyde-treated cells in the liver and spleen of the recipient rabbit. On the basis of comparison between the distribution of normal, electrosensitised and glutaraldehyde-treated normal cells over the scanning time period it is clear that the circulation of electrosensitised cells is not compromised by any modification resulting from the electrosensitisation procedure. The results suggest that the use of electrosensitised erythrocytes as a vehicle for payloads offers the advantage over competing technologies (e.g. liposome technology) in that they are not recognised by the reticulo-endothelial system, thereby offering prolonged circulation times in vivo.

In addition, when PEGylated normal erythrocytes are introduced into a recipient animal, it is found that their distribution during circulation over the scanning time examined is normal (FIG. 13, Panel D). In this case scan capture is more frequent as indicated, but the overall scan time again ranged from time zero to 20 minutes. This latter result suggests that PEGylation of erythrocytes does not cause damage which results in recognition of the modified cells by the reticulo-endothelial system and further suggests that if our carrier erythrocyte technology requires PEGylation for what ever reason, then it may be applied without negative consequence in terms of premature removal from circulation.

### Example 13

### The Effect of Pegylation on Circulation of Human Erythroyctes in the Rabbit

As mentioned above PEGylation of foreign entities aids in preventing recognition by macrophages of the reticulo-endothelial system. In order to determine whether or not PEGylation facilitates prolonged circulation of species-heterologous erythroyctes it was decided to examine circulation of normal, PEGylated normal and PEGylated electrosensitised human erythroyctes in rabbits using the ⁹⁹Tc-based monitoring method described above.

To this end, peripheral venous blood is obtained by venipuncture, and added to 10 ml blood collection tubes containing lithium heparin anti-coagulant. Donor phenotype is determined by agglutination with antisera from Lome Laboratories Ltd, (Twyford, UK). Aliquots (1 ml) of whole blood was added to Eppendorf tubes and centrifuged at 300 g in a centrifuge (Heraeus Biofuge pico), for 15 minutes at room temperature. Plasma and white cells are removed and the cell pellet is resuspended to 1 ml in cold (4° C.) phosphate buffered saline (PBS, Oxoid, Basingstoke, UK). Cells are washed at twice at 800 g for 3 minutes, in cold PBS. Pellets are resuspended to a final volume of 12 ml with cold PBS. Erythrocytes/red blood cells (RBC) are diluted 1 in 200 and counted with a haemocytometer. Peripheral venous blood collected as described previously is aliquoted in Eppendorf tubes and centrifuged at 800 g for 3 minutes at room temperature. Plasma is harvested and stored in Eppendorf tubes at room temperature until required. In cases where electrosensitisation and PEGylation are required, these were performed as described above for Example 12.

One ml of the prepared RBC (between 7-8×10⁸ c/ml) and 0.5 ml of autologous plasma are mixed and then injected into the technetium kit as per the kit protocol (Mallinckrodt UK Ltd, Bichester, UK). Following labelling, the contents of the kit are harvested and the cells washed 3 times in PBS at 800×g for 3 minutes. Supernatants are retained to check the levels of radioactivity. The final cell pellets re resuspended in 1 ml of PBS (with the exception of (+) EP cells which are resuspended in 0.5 ml of PBS) and the cells counted. The cells are then injected into the right ear of the rabbits (male or female New Zealand white rabbits, which weighed between 3 and 4 kg). Following injection, the fate of circulating RBC is monitored with a gamma camera. The accumulated images are analysed by specialised computer packages. The circulation time of RBC is analysed by monitoring circulating labelled erythroyctes in the heart.

Results 13

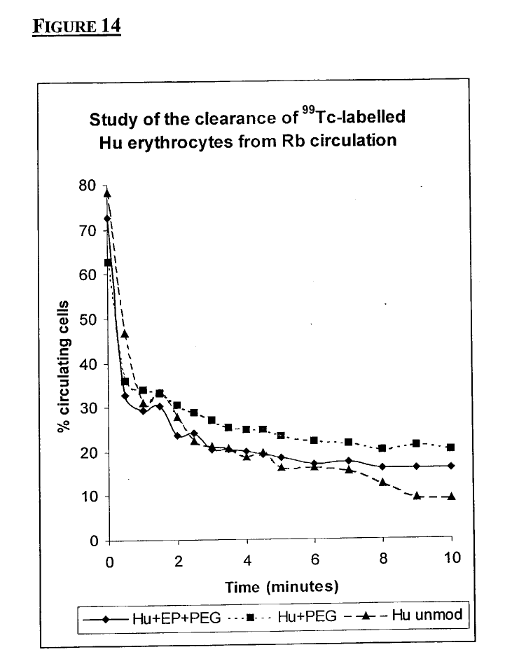

The results obtained from these experiments are shown in FIG. 14. When labelled normal, PEGylated normal and PEGylated electrosensitised cells are introduced into recipient rabbits, the proportion of labelled cells remaining in circulation drops to approximately 35% within 1 minute. However, at time intervals following that stage, the advantage associated with PEGylation becomes apparent particularly at 10 minutes. At 10 minutes the unmodified normal human cells continue to decrease in circulation whereas survival of the PEGylated normal and PEGylated human cells in circulation is enhanced.

The results suggest that if species-heterologous or modified autologous/homologous sensitised erythroyctes are to be employed as a delivery vehicle, recognition by the macrophages of the reticulo-endothelial system and consequential removal from circulation can, at least in part, be prevented by PEGylation.

### Example 14

Since the above studies demonstrate that sensitised rabbit erythroyctes are stable during circulation in vivo it was decided to confirm these results using an alternative labelling method and monitoring system.

It has been shown that erythrocytes may be conveniently labelled using the fluorogenic label PKH-26 which incorporates into the membrane of the cell (Selzak & Horan, 1989, Blood, 74, 2172-2172). Cells may therefore be introduced into a recipient animal and monitored using flow cytometry. If this system can be employed to monitor normal (autologous, heterologous rabbit) and electrosensitised rabbit cells during circulation in vivo it will circumvent problems associated with the relatively short half life of ⁹⁹Tc.

Cells are harvested and where required, electrosensitised as described above in Example 12. Cells are then PKH-26 labelled as follows:

1—Remove 5 ml of blood from rabbit ear vein

2—Wash twice to remove buffy coat in saline and process cells as required for treatment

3—Re-suspend cells in saline to give a packed cell volume of ˜0.25 ml

4—Centrifuge & remove supernatant

5—Add 1 ml of PKH-26 labelling kit buffer C (Sigma #MINI-26) & resuspend cells

6—To 1 ml of buffer C add 4 μl of 1 mM PKH-26 solution, mix well & add to cell suspension

7—Mix tube by gentle inversion for 5 minutes

8—Spin for 10′

9—Remove supernatant & wash 3 times in saline

10—Count cells & resuspend in 1 ml of saline

11—Verify cell labelling using flow cytometry

Following labelling cell preparations (0.5 ml packed cells) are injected into the rabbit ear vein (recipient animal weighs between 3 and 4 kg). At the indicated times 5 μl samples are collected into 1 ml of heparin-containing saline. Samples are subsequently analysed using flow cytometry and the percentage of labelled cells in circulation is determined.

Results 14

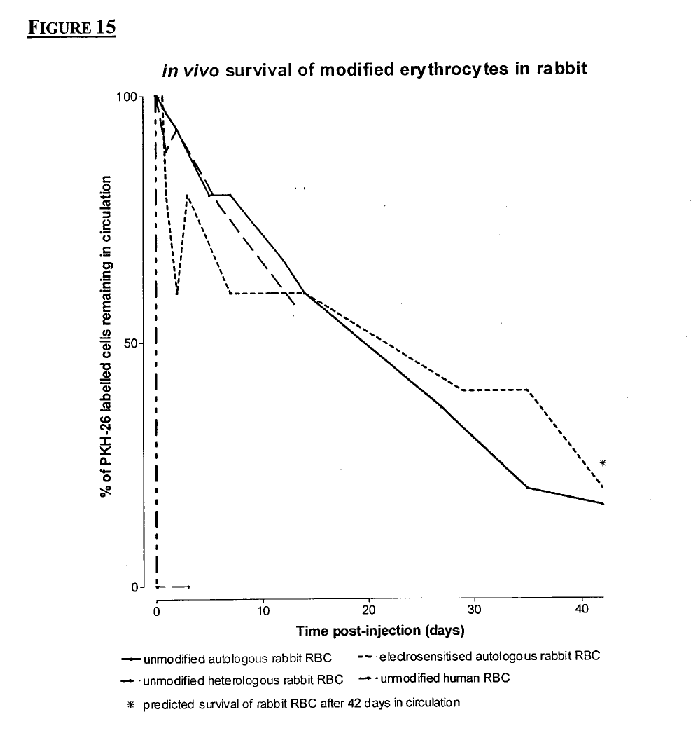

Rabbits are injected with PKH-26 labelled rabbit normal autologous, normal heterologous and electrosensitised cells. PKH-26 labelled human erythrocytes are used as a control for reticulo-endothelial scavanging. Samples are harvested at the indicated times and the labelled cells in circulation expressed as a percentage of the amount detected at time zero are detected by flow cytometry. The results obtained are shown in FIG. 15 and they demonstrate that PKH-26 labelled normal autologous and heterologous rabbit cells circulate normally (see FIG. 15 for predicted status of normal cells based on published T_{½} for normal rabbit erythrocytes [from Vomel & Platt, 1981, Mech. Ageing Dev. 17, 261-266]). In addition, PKH-26 labelled electrosensitised cells are also shown to circulate with pharmacokinetics similar to those exhibited by normal cells (FIG. 15). On the other hand human cells are rapidly cleared from circulation. The results confirm our earlier findings that electrosensitised cells circulate with pharmacokinetics similar to those exhibited by normal circulating rabbit cells.

### Example 15

### Survival of Loaded and Electrosensitised Erythroyctes in Vivo

As shown above, electrosensitised cells are stable during circulation in vivo. It is also of interest to determine whether or not cells which are electrosensitive and also processed through a loading protocol such as hypotonic dialysis loading, remain intact during circulation. It was therefore decided to produce ultrasound sensitive erythroyctes loaded with antibody (rabbit anti-human IgG) and introduce them into an animal. The cells are PKH labelled and their circulation in vivo is monitored using flow cytometry as described above. Rabbit erythrocytes are harvested and washed by centrifugation in PBS. Erythroyctes are loaded and sensitised by a modification of the procedure devised for human cells and described in detail as follows:

Buffers and Solutions:

PBS: Phosphate buffered saline tablets (Oxoid: Code BR14a) made up as per instructions

PBS-Glutathione: Above supplemented with 0.5 mM reduced glutathione

PBS-MgCl₂-Glutathione: PBS (above) supplemented with 4 mM MgCl₂ and 0.5 mM reduced glutathione.

Dialysis buffer (hyptonic): 12.5 mM K₂HPO₄/KH₂PO₄ containing 0.5 mM reduced glutathione, pH 7.4

PIGPA: 5 mM adenine, 100 mM inosine, 2 mM ATP, 100 mM sodium pyruvate, 100 mM glucose, 4 mM Mg Cl₂, 194 mM NaCl, 1.6 mM KCl, 35 mM NaH₂PO₄, pH 7.4

BAX/0.5 mM Glutathione: PBS (as above) supplemented with 5 mM adenosine, 5 mM glucose, 5 mM MgCl₂ and 0.5 mM reduced glutathione, pH 7.4.

Dialysis Tubing Preparation:

Dialysis Tubing: Spectra/Por® Membrane MWCO: 12-14,000 No.2

Flat Width: 10+/−1 mm

Diameter: 6.4 mm

Method:

Day one

1—Collect 10 ml of blood from rabbit ear vein into 10 ml heparinised tube

2—Transfer blood to 15 ml tube & spin at 3,000 rpm for 3′ at RT°

3—Remove plasma & buffy coat

4—Add equal volume of PBS, re-suspend & spin at 2,000 rpm for 15′ RT°

5- Repeat step 4

6—Aliquot 0.5 ml of packed cells plus 1 mg of Rb α-Hu IgG into the dialysis tubing.

7—Place tubing into a 3 ml electroporation cuvette & fill the cuvette with PBS-GSH.

8—Electrosensitise at RT°, 5 kV/cm, 3 μF, double pulse.

9—Remove dialysis tubing immediately & place into 100 ml of dialysis buffer on ice. Dialyse with stirring for 30′.

10—Transfer tubing to tubes containing 30 ml of PBS-MgCl₂ & incubate for 11′ at 37° C. with intermittent agitation of tubes.

11—Transfer the contents to 1.5 ml eppendorf tube & measure the volume. Add PIGPA to the tube at 1/10^{th} the volume & incubate at 37° C. for 30′.

12—Transfer to a 15 ml tube & bring the volume to 2 ml with Bax buffer at RT.

13—Centrifuge cells at 900 rpm for 5′.

14—Resuspend cells in 2 ml Bax buffer & store O/N at 4° C.

Day two

1—Following O/N storage centrifuge samples at 900 rpm for 5′ at 4° C.

2—Remove supernatant and resuspend to 4 ml with Bax buffer & wash twice at 900 rpm for 5′, followed by 1×1400 rpm centrifugation for 5′

3—Resuspend samples initially in 2 ml of Bax buffer & pool samples into a 15 ml tube on ice & remove a small sample. Adjust volume of the sample to give a cell concentration of 7×10⁸ cells/ml

4—Verify ultrasound sensitivity (1 MHz, 1.25 W/cm², 15 seconds)

The loaded and sensitised cells are then labelled with PKH-26 as described above and again ultrasound sensitivity is verified. In these experiments 1×10⁹ cells are injected into a rabbit (3 kg) which is anaesthetised by injection with a 0.3 ml Vetalar/0.2 ml Rompun mixture into the left ear vein. A 50 μl sample is taken prior to injection and placed in 450 μl of PBS containing heparin. Subsequent samples are taken at the times indicated in a similar manner post injection of labelled cells and samples are analysed by flow cytometry.

Results 15

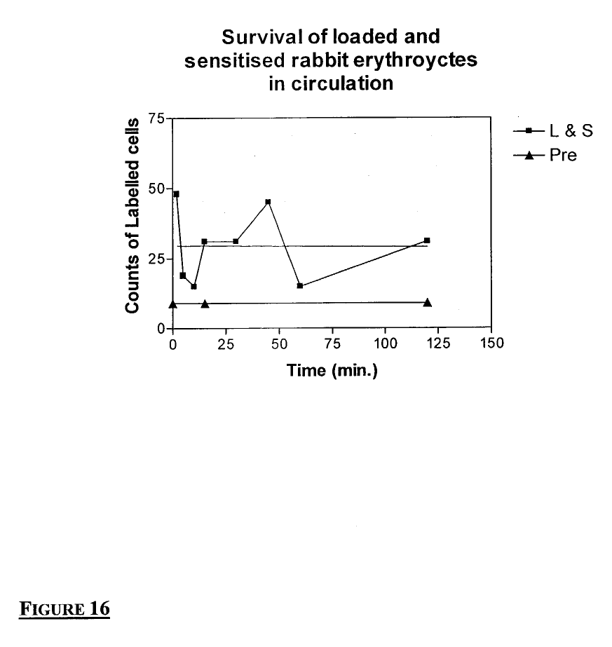

The results obtained are shown in FIG. 16 and they demonstrate that counts of labelled cells remain above background throughout the period examined. The line indicated by upright triangles is simply for reference to the counts detected in the blood sample taken prior to injection of the labelled cells. The results demonstrate that approximately 70% of the introduced cells remain at two hours. It should be noted that if these cells are being recognised by the reticulo-endothelial system, clearance should occur within 5 minutes. Since this does not occur the results clearly demonstrate that the loaded and sensitised vehicle is relatively stable during circulation in vivo.

### Example 16

### Ultrasound-Mediated Release of Payload from the Loaded, Sensitised Vehicle in a Circulating System At 37° C. and At High Hematocrit (Hct.)

In the above studies it is shown that human erythroyctes can be sensitised to low intensity ultrasound and ultrasound-mediated disruption and/or payload release can be achieved in vitro and in an ex vivo perfused rat kidney system. In all of those systems disruption and/or payload release is demonstrated at 7×10⁸ cells/ml which is approximately equivalent to a 5% Hct. In addition, those studies are performed at room temperature. Since Example 12 demonstrates that human cells are rapidly cleared from circulation in an animal model system it is of interest to demonstrate that sensitivity in terms of payload release can be retained at 37° C. and at higher Hct. It is also of interest to determine whether or not this occurs while the target cells are moving through a circulation system in much the same as those circulating in vivo.

To these ends human erythrocytes are harvested and loaded with anti-von Willebrand factor antibody as described for Example 10. Following sensitisation cells re mixed together with normal washed human cells in the proportions of one part 7×10⁸ cells/ml and four parts 4×10⁹ cells/ml. The mixture is introduced into a circulating system consisting of a cylindrical reservoir filled with PBS and maintained at 37° C. by circulation. The bottom of the cylinder consists of a light polyethylene sheet through which ultrasound is delivered. The blood is circulated through C-flex tubing (internal diameter 4 mm) which passes through the thermostated buffer and the target area of the C-flex tubing is positioned at a distance of 1.3 cm from the ultrasound-emitting head. Blood is circulated through the system at a rate of 14.5 ml/min. During exposure to ultrasound (5 W/cm² at 1 MHz for indicated times), samples are harvested from the system and supernatants are harvested by centrifugation. These are then assayed for antibody using an ELISA assay as described above. The control in these experiments consists of loaded and sensitised cells circulated through the system in the absence of ultrasound. It is also important to note that circulation of normal cells through the system while ultrasound is being delivered results in no apparent damage as determined by the lack of hemoglobin in supernatants following treatment.

Results 16

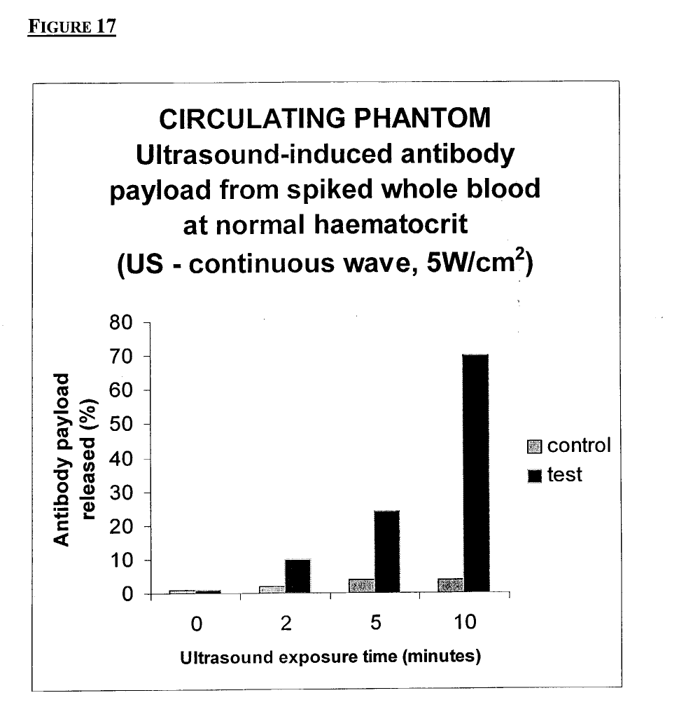

The results are shown in FIG. 17 and they demonstrate that detectable quantities of antibody are released from the vehicle between 2 and 5 minutes treatment with ultrasound. Little or no antibody can be detected in control samples which consist of the loaded and sensitised cells circulated through the system in the absence of ultrasound. The results demonstrate that the ultrasound-sensitisation phenomenon is intact at 37° C. and ultrasound-mediated release of payload is achieved at high hematocrit (Hct.) and in a mobile target system.

### Example 17

### Circulation of Payload and Detection Limits

Since it has been demonstrated in the above examples that loaded and sensitised rabbit erythrocytes are relatively stable in vitro it was decided to examine ultrasound-mediated release of payload in the rabbit. In these studies rabbit anti human IgG is chosen as the payload and quantified using an ELISA system based on recognition of human IgG. Prior to embarking on in vivo studies involving release of antibody from the vehicle, it was decided to examine circulation of the payload alone in order to demonstrate that it is not prematurely removed from circulation. This was also carried out in order to determine the detection limits of the antibody in plasma during circulation.

To this end rabbits (approx. 3 kg) are anaesthetised by injection with 0.3 ml Vetlar and 0.2 ml Rompun into the ear vein. Antibody is injected into the recipient rabbits at a concentration of 0.5 mg/kg. A 50 μl sample of blood is harvested prior to injection of antibody and mixed together with 450 μl of PBC containing heparin. Following administration of antibody 50 μl samples are harvested at the indicated times and mixed together with 450 μl of PBS containing heparin. Samples are then centrifuged at 2000 rpm using a microfuge and the supernatants are aliquoted into 150 μl quantities for storage at −80° C. Samples are analysed for antibody content using an ELISA method based on recognition of human IgG.

Results 17

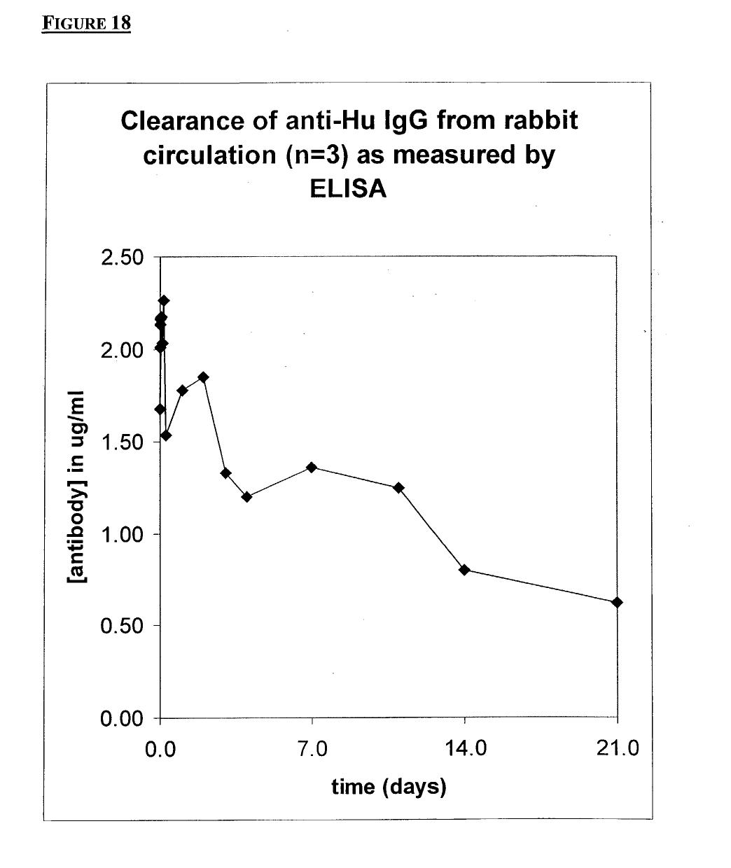

When harvested samples are analysed for rabbit anti-human IgG activity by ELISA the profile shown in FIG. 18 is obtained. The results demonstrate that the introduced antibody remains at detectable levels in circulation for up to 21 days and this would be well within the required time frame for ultrasound-mediated release studies. In addition the lowest detectable concentration of 0.6 μg/ml at day 21 suggests that loaded cells containing 100 μg of antibody are sufficient for the ultrasound-mediated release studies.

### Example 18

### Ultrasound-Mediated Release of Payload from the Erythrocyte Delivery Vehicle in Vivo

In order to demonstrate that ultrasound-mediated release of payload from the sensitised and loaded vehicle can be effected in vivo it was decided to sensitise and load rabbit erythrocytes with rabbit anti-human IgG. This is performed as described above for Example 15. Ultrasound sensitivity is confirmed prior to use and the cell population is shown to contain approximately 200 μg of antibody/ml of packed cells.

The recipient rabbit (3 kg) is anaesthetised by injecting 0.45 ml of Vetalar and 0.3 ml of Rompun sub-cutaneously into the back of the neck. Hair is removed from the abdominal area over the liver. Anaesthesia is maintained by placing the animal on 2% isofluorane delivered via a face mask. A pre-injection sample of blood (50 ml) is taken and placed in 450 μl of PBS containing heparin. 0.5 ml of packed cells are then injected into the animal through the ear vein. The animal is rested for 10 minutes and another sample of blood is harvested. Ultrasound contact gel is liberally applied to the shaven area to mediate contact with a 1 MHz ultrasound head. Six 4-minute ultrasound treatments are applied on continuous wave delivery at 4 W/cm² with each treatment interrupted by 30 seconds within which a sample of blood is harvested. Following the six treatments the animal is rested for 20 minutes and two blood samples are taken at 10 minute intervals. Treatment is re-initiated at 60 minutes again at 4 minute intervals with 30-second rest intervals within which blood samples are taken. The animal is again rested after six treatments for a period of 30 minutes during which samples of blood are harvested at 10-minute intervals. The control animal consists of an animal into which a similar quantity of cells are injected and ultrasound treatment is withheld. Both animals are euthenised using pentobarbitone following treatment. Supernatants are harvested by centrifugation from all samples and subsequently assayed by ELISA to detect circulating rabbit anti-human IgG.

Results 18

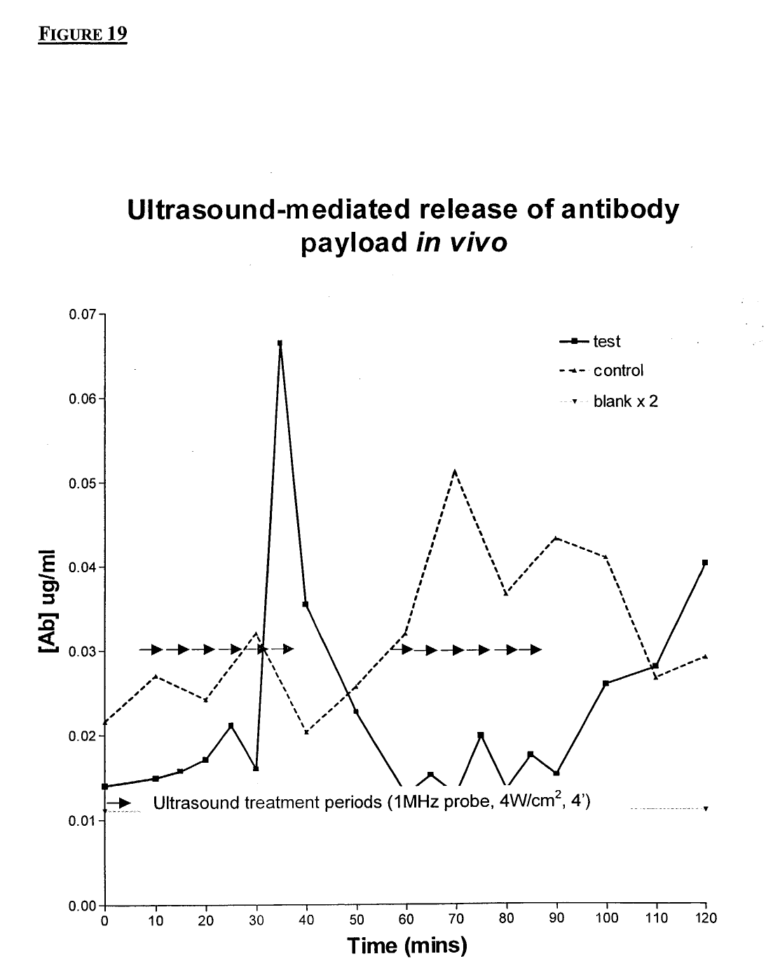

The results obtained during these experiments are shown in FIG. 19. Ultrasound treatments are indicated by the solid arrows and the dotted line traversing the lower portion of the curve represents the pre-injection background signal received from the ELISA. The results obtained from the ultrasound treated animal demonstrate the presence of antibody in circulation following 16 minutes treatment with ultrasound. After this period and during the rest period the amount of antibody in circulation decreases. However following the second phase of treatments initiated at 60 minutes the antibody concentration again begns to rise. In the control animal the level of antibody in circulation does not exhibit this pattern. It is interesting to note that the level of antibody in the first peak appearing in the ultrasound-treated animal accounts for approximately 16-20% of the total antibody contained in vehicle injected into the animal. This suggests that the remainder of antibody-containing vehicle supplies a sufficient reservoir for release as indicated by the second peak at 120 minutes. These results demonstrate ultrasound-mediated release of a payload from the sensitised and loaded vehicle in vivo.

### Example 19

### Ultrasound Mediated Release of Payload Release in vivo in Pig, and Demonstration that a Circulating Cell Remains Sensitised

The objective of this experiment is to demonstrate that the relevant peptide is released by ultrasound from the vehicle in an in vivo model. Secondly, we investigate whether the loaded cells collected from the circulation still retain ultrasound sensitivity in an in vitro system. The presence of any in vivo repair processes to the loaded vehicle may be identified.

Whole blood from pig is collected in heparinised containers. Cells are washed once in PBS at 700 g for 5 min, and once in buffer 1 at 700 g for 5 mins. The composition of buffer 1 (isoosmotic PBS) is as follows: pH7.4K₂H/KH₂ phosphate buffer with 150 mM NaCl (8.76 g/l); check and adjust pH with 1M NaOH.

The cells are retained as a packed cell volume and 0.4 mg of fluorescein-labelled HIV TAT (Alta Biosciences, Edgbaston, Birmingham) is added for every 7×10⁸ cells. The mixtures placed in dialysis tubing (1000 Da MW tubing, Spectro-Por, Spectrum Inc.) for 60 min at 37° C. Cells are then dialysed against buffer 2 for one hour at 4° C. The composition of buffer 2 (dialysis buffer) is as follows: pH7.4K₂H/KH₂ phosphate buffer; check and adjust pH with 1M NaOH. Membranes are then placed into mBAX at 37° C., and dialysed for one hour.

Cells are harvested from the dialysis membranes and washed three times in mBAX buffer at 170 g for 15 minutes at room temperature. Cells are resuspended at 7×10⁸ in mBAX and stored at 4° C. overnight.

The next day, cells are washed as described in Example 1 (second procedure) of PCT/GB00/03056. The cell concentration is adjusted to 7×10⁸ cells/ml.

The test system comprises two healthy, mature pigs of a crossbreed type (Large While ×Landrace) of the male sex at least four weeks of age, each weighing 10 kg. Venous puncture of the jugular vein of each animal enables 35 mls of whole blood to be available for processing i.e., electrosensitisation and dialysis loading with fluorescently labelled HIV-TAT fragment. Anaesthesia is induced by injection of pentobarbitone at a dose rate of approx. 25 mg/kg bodyweight (Sagatal (Merial)). The exterior ileal vein is catheterised and fitted with a 3 way tap, for sample adminstration and sampling. Preadministration samples are collected, prior to the test system receiving the processed packed cells, by slow intravenous injection (5 ml).

In one of the subjects, after 60 min, ultrasound is applied to the jugular/carotid region of the neck at 6 W/cm² (pulsed wave; 35%) (RICH-MAR CRM-1 machine fitted with a 1 MHz head, for 3×10 min bursts, with a 1 minute rest between each 10 minute burst. The surface of this area is liberally covered with Alpha Lube gel (Ultrasonic Scanning gel, BCF Technology Ltd) before application of the head.

Samples are collected at the time periods shown, and analysed using flow cytometry, where cells counts of the loaded cell population are assessed.

Additional samples are collected, 10 minutes following cell administration to the animal, for application to the in vitro circulating model. Samples are collected 10 minutes following cell administration, from the circulating system, and assessed using flow cytometry, where cells counts of the loaded cell population are analysed

Results 19

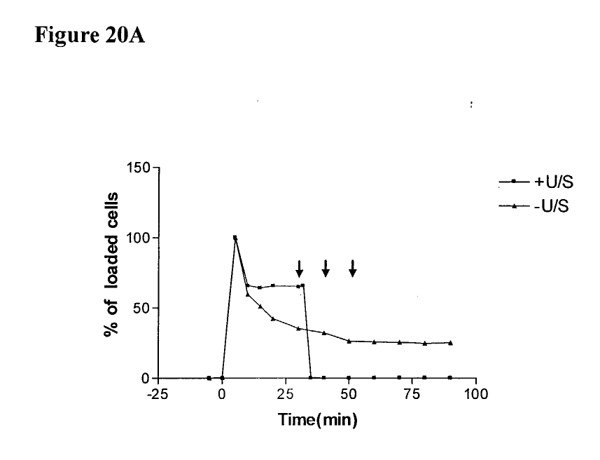

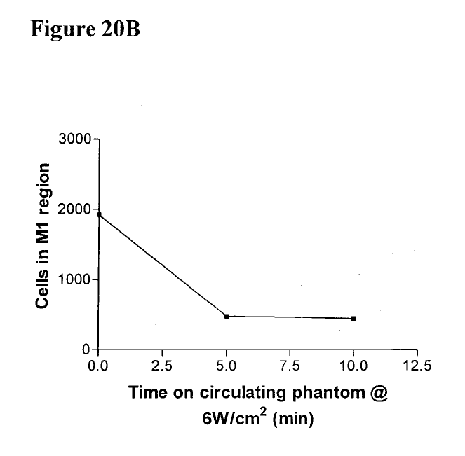

FIG. 20A demonstrates that a clear increase in percentage loaded cells coincides with administration of loaded vehicle into the animal. In both subjects, cell number decreases quite significantly, between 5 and 10 minutes following administration. Spiking of a comparable volume of loaded cells into whole blood however would suggest that the 5-minute sample may not have been an accurate representation, with insufficient dilution of the loaded cells.

In the control animal, to which no ultrasound is applied, a gradual decline in labelled cell number is observed. In contrast, the effect of ultrasound on loaded cells in vivo is pronounced, and a dramatic decrease is shown between 2 and 5 minutes of ultrasound treatment at 6 W/cm², pulsed wave; 35%.

FIG. 20B illustrates that samples collected 10 minutes following cell administration to the animal, for application to the in vitro circulating model, still show a decrease in loaded cell number with ultrasound treatment. This would suggest that in vivo repair processes during circulation are negligible, and the loaded vehicle still demonstrates ultrasound sensitivity.

### Example 20

### Materials and Methods

In this and the following Examples, peptides penetratin, HIV-TAT and VP22 are loaded into electrosensitised erythrocytes. These Examples show that the loaded peptides may be released in vivo, and delivered to cells.

Buffers

Bax buffer and Bax modified buffer (mBax) are described in Bax et al (1999), Clinical Science 96, 171-178 and have the following compositions: Bax-modified buffer (mBAX: PH 7.4; 2.68 mM KCl, 1.47M KH₂PO₄, 136 mM NaCl, 8.1 mM Na₂HPO₄, 5 mM glucose, 5 mM adenine, 5 mM MgCl₂. Check and adjust pH with 1M NaOH); Bax buffer (BAX: PH 7.4; 2.68 mM KCl, 1.47M KH₂PO₄, 136 mM NaCl, 8.1 mM Na₂HPO₄, 5 mM glucose, 5 mM adenosine, 5 mM MgCl₂. Check and adjust pH with 1M NaOH).

Payload

The penetratin payload comprises a FITC-Penetratin conjugate, having the following sequence: Fluorescein-RQIKIWFQNRRMKWKKC (custom made by Altabioscience, Southampton, UK). The HIV-TAT fragment has the following sequence: Fluorescein-GRKKRRQRRRPPQC-amide (2181.5 Da). VP22 as used here has the following sequence: NAATATRGRSAASRPTERPRAPARSASRPRRPVEC-amide. VP22 is obtained from Alta Biosciences, Edgbaston, Birmingham.

Loading of Rabbit Red Blood Cells

Whole blood from rabbit is collected in heparinised containers and cells are washed and sensitised. The cell concentration is adjusted to 1.5×10⁹ and fluorescein-labelled penetratin, HIV-TAT fragment and VP22 are added at the indicated concentrations (in PBS) and mixtures are incubated for 30 min at 37° C.

The mixtures are then centrifuged at 700 g for 5 minutes and the cells are resuspended with PBS-Mg-Glucose (rabbit and mouse) or mBAX (human and pig), and subsequently washed twice. Uptake of peptide is monitored by analysis on flow cytometry where this uptake of the fluorescent peptide is indicated by a shift to the right on the flow cytometry profiles.

The results show that increasing concentrations of HIV-TAT, penetratin and VP-22 results in increasing shifts to the right on the flow cytometry profiles. In each case the progressive shift the right is indicative of peptide uptake by the sensitised carrier vehicle.

Loading of Pig and Human Red Blood Cells

Whole blood from pig is collected in heparinised containers and cells are washed and sensitised as described in Example 1 (second procedure) of PCT/GB00/ 03056. The cell concentration is adjusted to 7×10⁸.

Whole blood from human is collected in heparinised containers and cells are washed and sensitised as described in Example 1 (second procedure) of PCT/GB00/03056.

Cells are washed once in PBS at 700 g for 5 min, and once in buffer (isoosmotic PBS: pH7.4K₂H/KH₂ phosphate buffer, with 150 mM NaCl; 8.76 g/L; check and adjust pH with 1M NaOH) at 700 g for 5 mins. The cells are retained as a packed cell volume and fluorescein-labelled HIV-TAT fragment (Alta Biosciences, Edgbaston, Birmingham) is added to the packed cell volume at the indicated concentrations (expressed as mg/ml peptide to 7×10⁸ cells/ml) and mixtures placed in dialysis tubing (1000 Da MW tubing, Spectro-Por, Spectrum Inc.,) for 60 min at 37° C. Cells are then dialysed against buffer 2 (dialysis buffer: pH7.4K₂H/KH₂ phosphate buffer; check and adjust pH with 1M NaOH) for one hour at 4° C. Membranes are then placed into mBAX at 37° C., and dialysed for one hour.

Cells are harvested from the dialysis membranes and washed three times in mBAX buffer at 170 g for 15 minutes at room temperature. Cells are re-suspended at 7×10⁸ in mBAX and stored at 4° C. overnight.

The next day, cells are washed and sensitised as described in Example 1 (second procedure) of PCT/GB00/03056, and the cell concentration is adjusted to 7×10⁸ cells/ml.

Uptake of peptide is monitored by analysis on flow cytometry where this uptake of the fluorescent peptide is indicated by a shift to the right on the flow cytometry profiles.

Spiking of Loaded Red Blood Cells into Whole Blood

Erythrocytes loaded with HIV-TAT fragment from human, rabbit, pig and mouse (as described above) are spiked (1%) into whole blood of the corresponding species. Stability at 37° C. and 4° C. is assessed for up to 24 hours using flow cytometry, where cells counts of the loaded cell population are analysed against time.

Tissue Mimicking System

Peptide-loaded cells are tested for sensitivity to low intensity ultrasound in a tissue mimicking system. TMM is a tissue mimicking material which attenuates ultrasound in the same manner as a soft tissue. The TMM chosen for this work is described in Madsen et al. (1998, Ultrasound Med. & Biol., 24, 535-542) and following preparation, care is taken to ensure that the material has a density of 1.03 g/ml).

In all cases, the peptide-loaded cells are shown to be preferentially sensitive to low intensity ultrasound (100% lysis when treated with ultrasound at 3 W/cm² and at 1 MHz) in such a tissue mimicking system.

Circulating Phantom

A circulating phantom system assays the ultrasound sensitivity of cells in a context which mimics a physiological environment. The release of a payload when treated with ultrasound may therefore be monitored in a system which imitates a circulatory system.

Cells are spiked into whole blood (approximately 2%) and circulated in a physiological buffer, at a flow rate which is similar to the central venous flow rate (approximately 15 ml/min) of a relevant organism, and at a temperature which is close to or identical to the body temperature of the animal in question.

The particular circulating phantom system as used in the Examples described below comprises a bath maintained at a suitable temperature, circulating means in which red blood cells may be circulated, and a ultrasound source. The ultrasound source comprises an ultrasound head which in use is placed adjacent to a wall of the bath. A portion of the circulating means is placed inside the bath, and the ultrasound head transmits ultrasound energy across the wall of the bath and the wall of the tubing to the red blood cells maintained in there.

The bath is maintained at 37 degrees C. (or any other suitable temperature) by an immersion heater and a thermostat. The bath contains water or a buffer such as PBS, although any liquid which has adequate temperature buffering capacity may also be used. The walls of the bath are constructed of for example plastic sheeting, although at least a portion of one or more walls should be constructed of a material which substantially allows the passage of ultrasound preferably without significant attenuation. The bath may therefore comprise a window in one of the walls allowing passage of ultrasound. A suitable ultrasound transmitting material includes builders plastic obtainable from a hardware store.

The circulating means enables red blood cells to be moved across the ultrasound field and enables exposure of cells to ultrasound. The circulating means preferably comprises tubing, for example ordinary laboratory plastic tubing. The tubing comprises at least a portion which is capable of transmitting ultrasound, and may be transparent or translucent to visible light. The tubing as used in the Examples comprises a section of ultrasound transmitting material (C-Flex™ tubing, made by Cole Parmer, UK) linked to a section of laboratory tubing. This is inserted into a peristaltic pump to drive the cells around the tubing. The bottom of the peristaltic tubing which acts as the target vessel is situated at 1.3 cm above the head of the probe.

In a preferred embodiment, the bath is cylindrical and the bottom of the bath consists of a light polyethylene sheet through which ultrasound is delivered. The blood is circulated through C-Flex™ tubing (internal diameter 4 mm) which passes through the thermostated buffer and the target area of the C-flex tubing is positioned at a distance of 1.3 cm from the ultrasound-emitting head. Blood is circulated through the system at a rate of 14.5 ml/min. During exposure to ultrasound (for example, 5 W/cm² at 1 MHz for indicated times), samples are harvested from the system and supernatants are harvested by centrifugation.

A protocol for initial equipment set-up is as follows: 1. Unscrew the bolts from the bottom and separate the top and bottom plates, and grease the rubber seals on the inside of each plate with Vaseline; 2. Cut a piece of builder's polythene to size and place tightly over the bottom plate. Replace the top plate and line up the screw holes. Replace screws to hold the polythene in place, making sure that the polythene remains as taut as possible; 3. Place some water into the unit to make sure that it is water tight. If not, unscrew the device and try again. 4. Place tubing onto water inlet and outlet on the CP and connect to the pumps. 5. Ensure that the ultrasound probe head is level, and securely held by a clamp. Place ultrasound gel on the probe head. 6. Clamp the CP chamber to a stand and place over the probe head—keep probe head as central as possible, making sure there are no air bubbles between the probe head and the surface of the polythene, and making sure that the surface is level. 7. Secure in place. 8. Place the two free ends of the inlet and outlet tubes into a water bath, set at 40° C. This will allow temperature in the CP to stay at approximately 37° C. Verified with a thermometer. 9. Switch the pumps on and allow the water to circulate—check the temperature and that the water level remains constant, at approximately 1 cm below the inlet pipe.

Sample Set-up is as follows: 1. Clamp on sample bar containing peristaltic tubing. Set the bottom of the tubing to 1.3 cm over the centre of the probe head. 2. Flush out the tubing with 20 mL PBS solution. 3. Once the tubing is clean, force out the PBS solution with air. 4. Immerse one end of the peristaltic tubing into 3 mL of freshly collected, washed whole blood (sample coming from same animal as loaded cells). 5. Slowly, pull the sample through the peristaltic tubing using the peristaltic pump controlling the flow, ensuring no air is allowed into the system. 6. Adjust the flow through the tubing to 15 mL/min. The peristaltic pump is marked with this flow. 7. Allow 15 minutes for the system to equilibrate to the temperature of the CP chamber. 8. Remove 0.6 mL of blood from the system. This will act as a control. 9. To the circulating blood, add 0.6 mL of loaded cells prepared according to the respective loading method, at 7×10⁸ c/mL. This corresponds to a 2% spike. 10. The system is now ready to be used for application of ultrasound.

### Example 21

### A. Effect of Ultrasound on Non-Electrosensitised HIV-TAT Loaded Vehicle in vivo in Pig (Jugular Region)

The objective of this experiment is to demonstrate that a loaded vehicle which has not been electrosensitised does not release its loaded components in vivo.

Red blood cell collection, preparation, dialysis loading, the test system and ultrasound administration are as described above in the preceding Example, with the exception that electrosensitisation does not take place.

Samples are collected at the time periods shown, and analysed using flow cytometry, where cells counts of the loaded cell population are assessed.

Results 21A

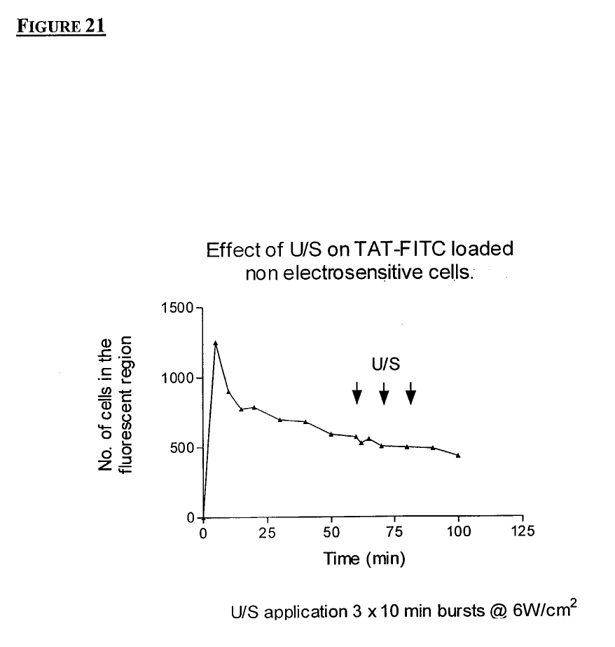

FIG. 21 demonstrates that a clear increase in number of loaded cells coincides with administration of loaded vehicle into the animal.

A gradual decline in labelled cell number is observed, prior to the administration of ultrasound, and during and after the 3×10 min bursts, under the parameters used, no effect on cell number could be observed. This would suggest that the as the cells are not resensitised, they are not receptive to ultrasound mediated lysis and subsequent release of payload, as would be predicted.

### Example 21B

### Effect of Ultrasound Targeting of the Jugular Region of a Pig Animal Model, on Kinetics of Payload Release in Vivo

The objective of this experiment is to establish kinetics of payload release, when ultrasound is applied to the jugular region of the animal model.

The test system comprises one animal, a description of which including blood collection, loading conditions, anaesthesia and sample collection conditions has been provided in the above Examples, with the following modifications.

After 30 minutes, ultrasound is applied to the jugular region of the neck at 6 W/cm² (pulsed wave; 35%) (RICH-MAR CRM-1 machine fitted with a 1 MHz head, for a 8×1 min applications, each minute application followed by 4 minutes rest period. The surface of this area is liberally covered with Alpha Lube gel (Ultrasonic Scanning gel, BCF Technology Ltd) before application of the head.

Samples are collected at the time periods shown, and analysed using flow cytometry, where cells counts of the loaded cell population are assessed.

Results 21B

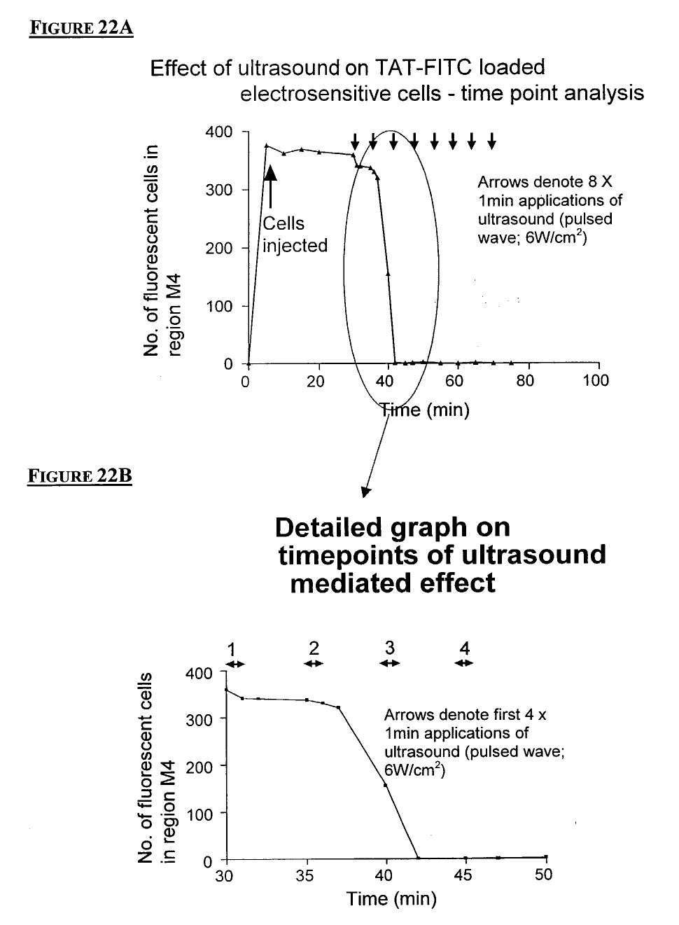

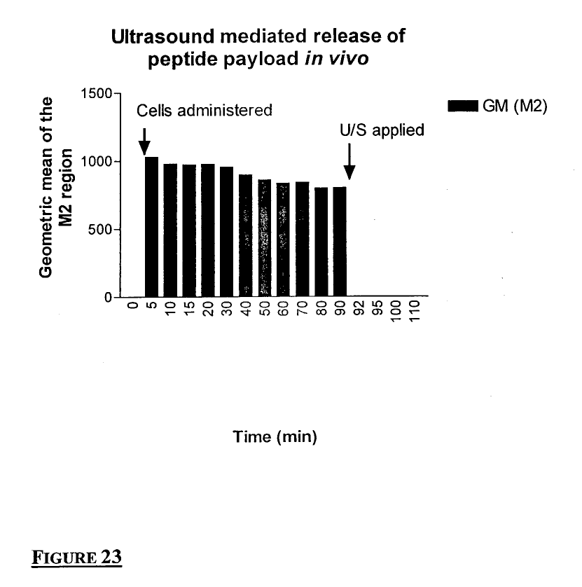

FIG. 22A (overall profile) demonstrates that following sample administration to the animal, the cells remain stable until the onset of ultrasound application. In the interim period of 2 and 3 minutes following ultrasound treatment, the number of loaded cells decreases completely.

FIG. 22B (detailed graph) shows that the 1^{st} 1-minute ultrasound application does not have any direct effect on loaded cell number. The decrease in loaded cell number occurs directly after the 2^{nd} 1-minute application, and this cumulative effect continues throughout the 3^{rd} application of ultrasound until no loaded cells remain.

### Example 22

### Effect of Ultrasound Targeting of the Hepatic Artery Region of a Pig Animal Model on Subsequent Release of Payload Release in Vivo

The objective of this experiment is to demonstrate that the relevant peptide can be released quickly from the vehicle, by ultrasound targeting of the hepatic artery region in an in vivo pig model.

The test system comprises one animal description of which including blood collection, loading conditions, anaesthesia and sample collection conditions have been described above.

After 90 min, ultrasound is applied to the hepatic artery/posterior vena cava/hepatic portal vein region of the neck at 6 W/cm² (pulsed wave; 35%) (RICH-MAR CRM-1 machine fitted with a 1 MHz head, for a 4 min application. The surface of this area is liberally covered with Alpha Lube gel (Ultrasonic Scanning gel, BCF Technology Ltd) before application of the head.

Samples are collected at the time periods shown, and analysed using flow cytometry, where cells counts of the loaded cell population are assessed.

Results 22

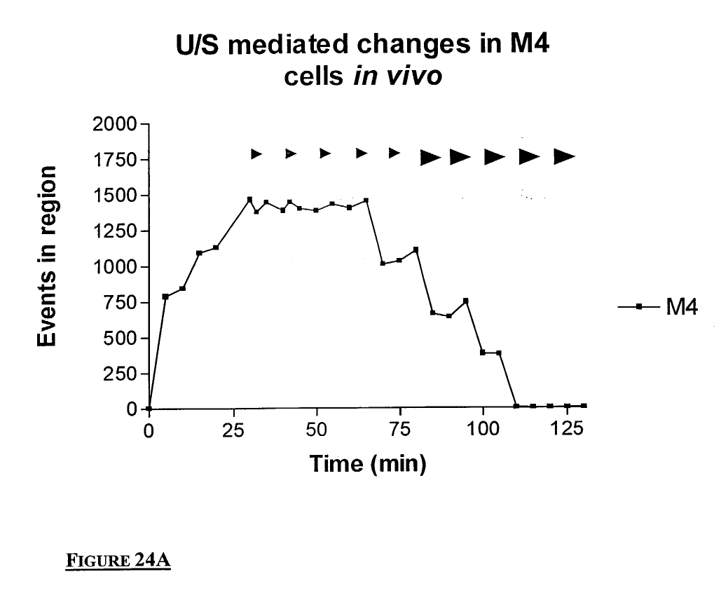

FIG. 23 demonstrates that a clear increase in loaded cell number as determined by flow cytometry (y axis) coincides with administration of loaded vehicle into the animal. The cells retain their sensitivity for 90 mins, where ultrasound is applied. Almost immediately, the loaded cells disappear, before the sample collected at 92 mins (i.e. 2 mins ultrasound treatment).

### Example 23

### Effect of Ultrasound Targeting One of the Kidneys of a Pig Animal Model on Subsequent Release of Payload Release in Vivo

The objective of this experiment is firstly to demonstrate that the relevant peptide can be released in a time dependant, pulsatile manner, when ultrasound is applied to the kidney (cortical region) of the pig animal model. Secondly, uptake of the labelled payload into surrounding tissue cells is observed.

The test system comprises one animal, a description of which including blood collection, loading conditions, anaesthesia and sample collection conditions has been provided in the above Examples, with the following modifications.

After 30 mins, ultrasound is applied to the cortical region of the right kidney at 6 W/cm² (pulsed wave; 35%) (RICH-MAR CRM-1 machine fitted with a 1 MHz head), with two sequential and different sets of ultrasound conditions. The 1^{st} set consists of 5 cycles each of 30 seconds with a 4 min 30 sec rest between cycles, while and the 2^{nd} set consists of 5 cycles each of 1 minute with a 4 min rest between cycles. The surface of this area is liberally covered with Alpha Lube gel (Ultrasonic Scanning gel, BCF Technology Ltd) before application of the head.

Samples are collected at the time periods shown, and analysed using flow cytometry, where cells counts of the loaded cell population are assessed.

The treated and non-treated kidneys are fixed in 4% paraformaldehyde for subsequent wax embedding. Sections (10 μM) are viewed under the fluorescent microscope to assess the amount of labelled peptide localised inside the cells.

Results 23

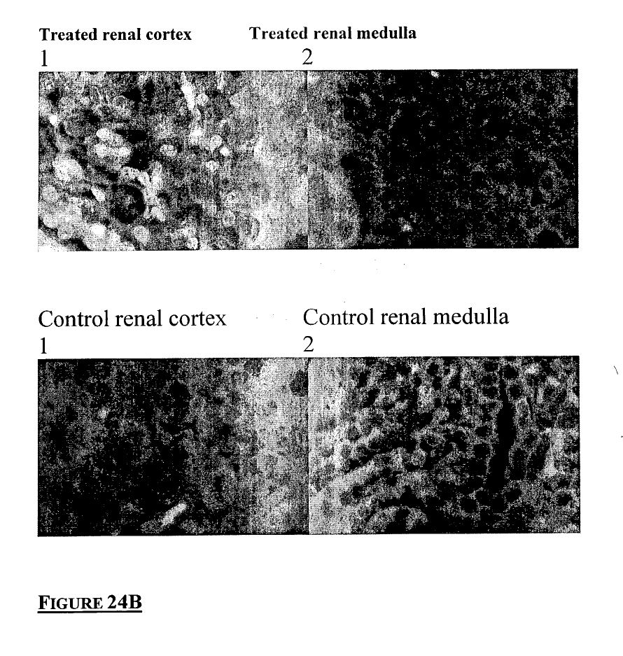

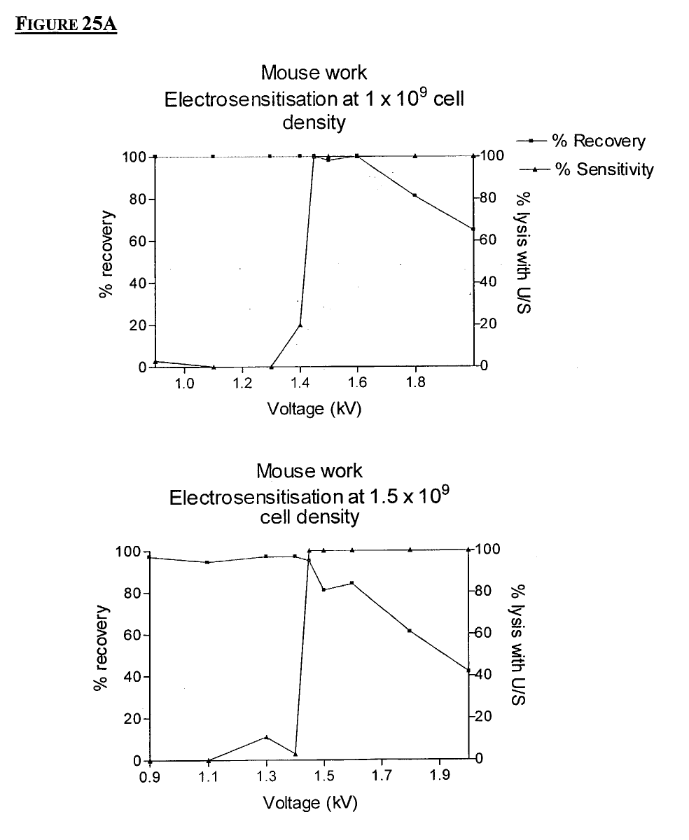

FIG. 24A demonstrates that the loaded vehicle retains a high level of stability in vivo, which remains constant prior to ultrasound application. Across the duration of 2 minutes treatment there does not appear to be any ultrasound mediated effect. However, after the ₄^{th} burst for 30 seconds, there is a stepwise decrease in loaded cell number and this cumulative effect continues. This indicates that the payload is being released in a pulsatile, stepwise or discontinuous manner.

FIG. 24B illustrates the ultrasound mediated localisation of FITC-labelled TAT in the treated kidney compared to the control organ. This clearly exhibits an enhanced localisation and uptake at the treated area, indicating that the peptide is released at the site of ultrasound treatment, and subsequently taken up by the cells in close proximity.

The following three Examples demonstrate optimal electrosensitisation and loading of FITC-labelled HIV-TAT into murine erythrocytes, ultrasound mediated release of payload in whole circulating mouse blood in vitro, and ultrasound mediated pulsatile release and localised uptake in a murine in vivo model.

### Example 24

### Production of an Optimally Electrosensitised Murine Erythrocyte Which is Loaded with the Peptide HIV-TAT

Whole blood from mouse is collected in heparinised containers and the cells washed as described in the general protocols set out in Example 1 (second procedure) of PCT/GB00/03056.

Optimal conditions for cell concentration, electrosensitisation voltage and pulse number are established. Cells are washed once in PBS-Mg at 700 g for 5 min, and once in buffer 1 at 700 g for 5 mins. The cells are retained as a packed cell volume and fluorescein labelled HIV TAT (Alta Biosciences, Edgbaston, Birmingham) is added to the packed cell volume at a concentration of 0.04 mg/ml (expressed as mg/ml peptide to 7×10⁸ cells/ml) Mixtures are placed in dialysis tubing (1000 Da MW tubing, Spectro-Por, Spectrum Inc.,). Cells are then dialysed against buffer 2 for one hour at 4° C. Membranes are then placed into mBAX at 37° C., and dialysed for one hour.

Cells are harvested from the dialysis membranes and washed three times in mBAX buffer at 170 g for 15 minutes at room temperature. Cells are resuspended at 7×10⁸ in mBAX and stored at 4° C. overnight. Cell recovery and sensitivity are assessed as markers by which conditions for an optimally sensitised and loading murine erythrocyte is obtained.

Loading of peptide into the erythrocyte is monitored by analysis on flow cytometry where the uptake of the fluorescent peptide is indicated by a shift to the right on the flow cytometry profiles.

Results 24

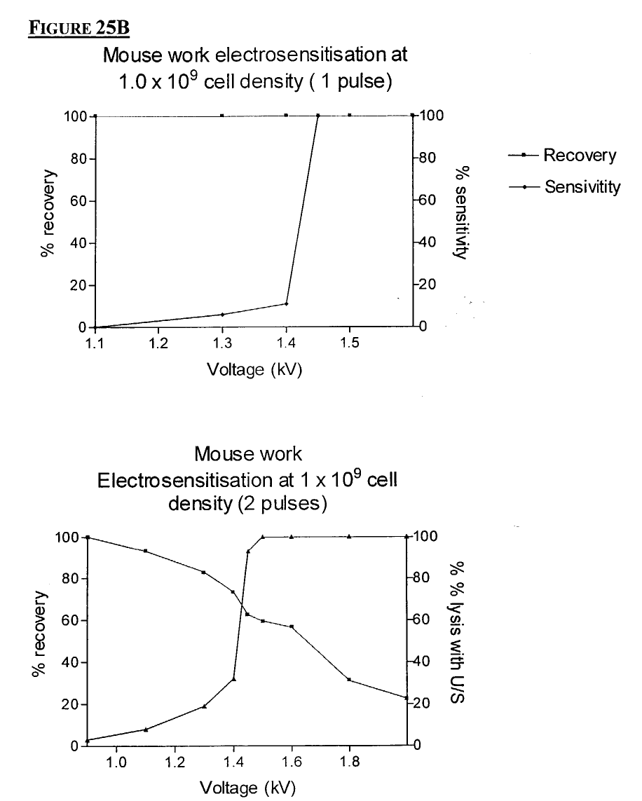

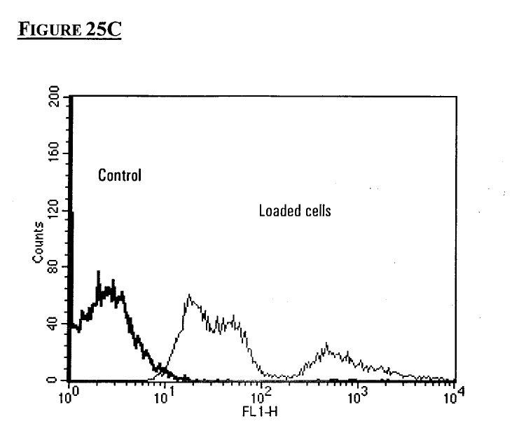

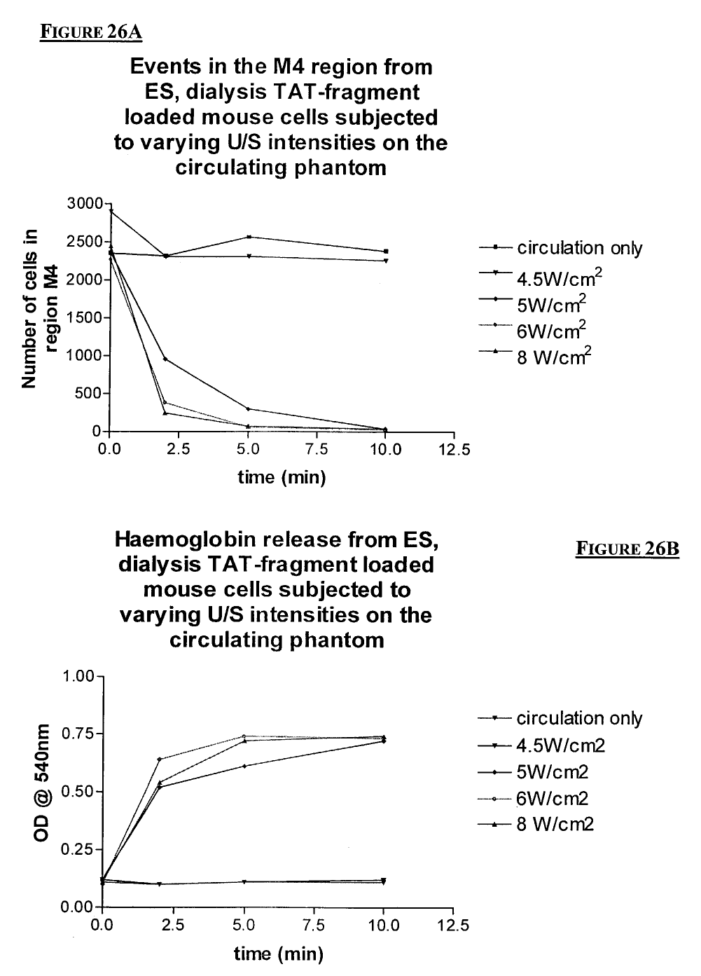

The results are shown in FIG. 25A and FIG. 25B, where a cell density of 10×10⁸ and 1 pulse at 1.45 kV elicit optimal cell recoveries and ultrasound sensitivity.

Peptide loading into the erythrocyte is shown a shift to the right on the flow cytometry profiles (FIG. 25C). The progressive shift the right is indicative of peptide uptake by the sensitised carrier vehicle

The peptide-loaded cells are shown to be preferentially sensitive to low intensity ultrasound (100% lysis when treated with ultrasound at 3 W/cm² and at 1 MHz using the TMM system as described in WO 01/07011). These results demonstrate that HIV-TAT may be dialysis loaded into electrosensitised mouse erythrocytes, producing a carrier which is sensitive to ultrasound.

### Example 25

### Ultrasound Mediated Release of Payload in Whole Circulating Mouse Blood in Vitro

The objective of this experiment is to demonstrate that the relevant peptide could be released by ultrasound from a loaded mouse erythrocyte in an in vitro circulating model, at 37 C., 1.3 cm from the ultrasound probe, spiked into whole blood. From this, ultrasound parameters may be established for further use in an in vivo system.

Mouse erythrocytes, electrosensitised and dialysis loaded with HIV-TAT are spiked (2.5%) into whole blood of the same animal. A 3 ml sample is then applied to the circulating phantom model at 4.5-6 W/cm² (pulsed wave; 35%) for 15 min, and 100 μl samples collected for the circulating system every 5 min. Any ultrasound mediated decrease in loaded erythrocytes is demonstrated by loss of cells on the flow cytometer.

Haemoglobin levels in the supernatants of the collected samples are determined by measuring the absorbance at 540 nm using a spectrophotometer.

Results 25

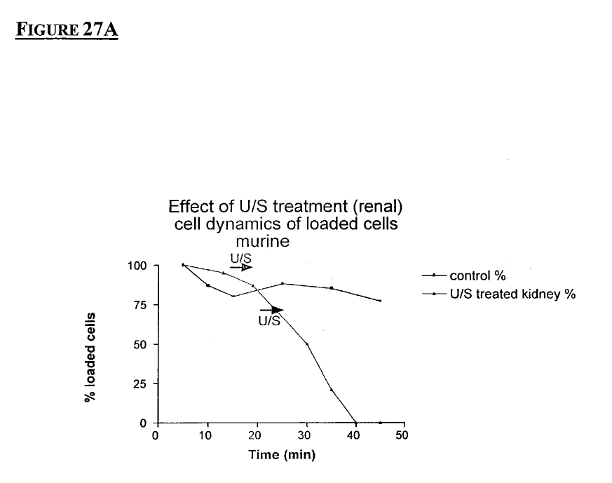

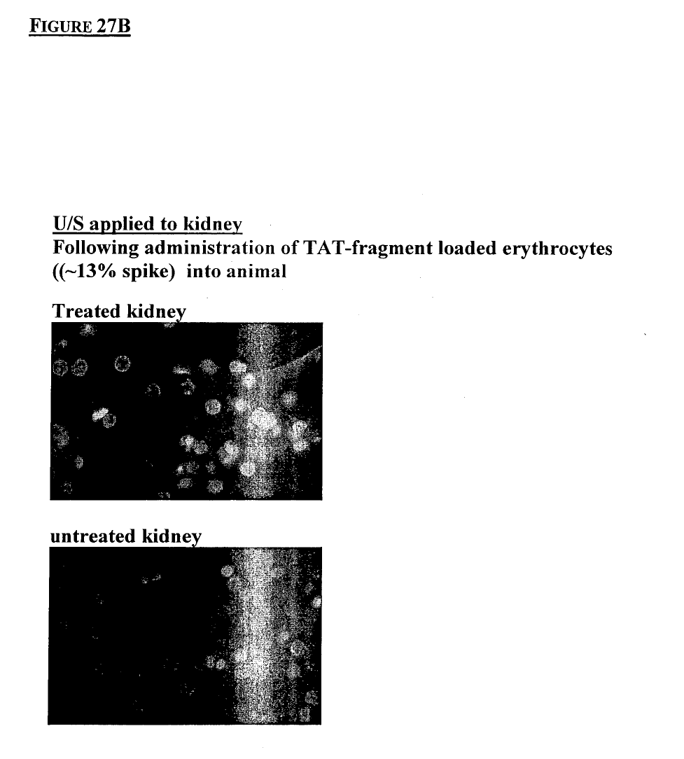

FIG. 26A demonstrates that with the parameters used, an ultrasound intensity of 4.5 W/cm² confers negligible effects on the number of loaded cells in whole blood. At 5-6 W/cm² a decrease in the number of loaded cells occurs after 2 min.

FIG. 26B demonstrates haemoglobin release at the various ultrasound intensities shows that release of this cell lysis marker mirrors the loss of labelled cells, showing that these cells are being targeted by ultrasound. These results show that in the in vitro circulating system, the loaded vehicle spiked into whole blood, is sensitive to ultrasound.

### Example 26

### Ultrasound Mediated Release of Payload in vivo from Mouse Erythrocytes

The objective of these experiments is to demonstrate firstly that the relevant peptide payload can be released in a pulsatile manner from the vehicle, in the context of an in vivo murine environment.

On release from the vehicle, it has been demonstrated that the peptides are capable of trafficking into target cells. In terms of exploitation in this invention, the functionality of the peptide is used to traffic into and beyond the vascular endothelium. Therefore, subsequent uptake of fluorescently-labelled peptide into endothelial cells can be investigated in an in vivo model.

The test system comprises two male Swiss To mice (8-12 weeks). Anaesthesia is induced by inhalation with isofluorane and maintained under 2% isofluorane with a flow rate of 21 oxygen/min). Administration of loaded packed cells (200 μl) and sampling ( 1 μl) is carried out from the tail veins (one for each).

In one of the subjects, after 15 min, ultrasound is applied directly to the cortical region of the kidney at 6 W/cm² (pulsed wave; 35%) (RICH-MAR CRM-1 machine fitted with a 1 MHz head, for 2×5 min bursts, with a 5 minute rest between bursts. The surface of this area is liberally covered with Alpha Lube gel (Ultrasonic Scanning gel, BCF Technology Ltd) before application of the probe head.

Samples are collected at the time periods shown, and analysed using flow cytometry, where cell counts of the loaded cell population are assessed.

The treated and non-treated kidneys are fixed in 4% paraformaldehyde for subsequent wax embedding. Sections (10 μM) are viewed under the fluorescent microscope to assess the amount of labelled peptide localised inside the cells.

Results 26

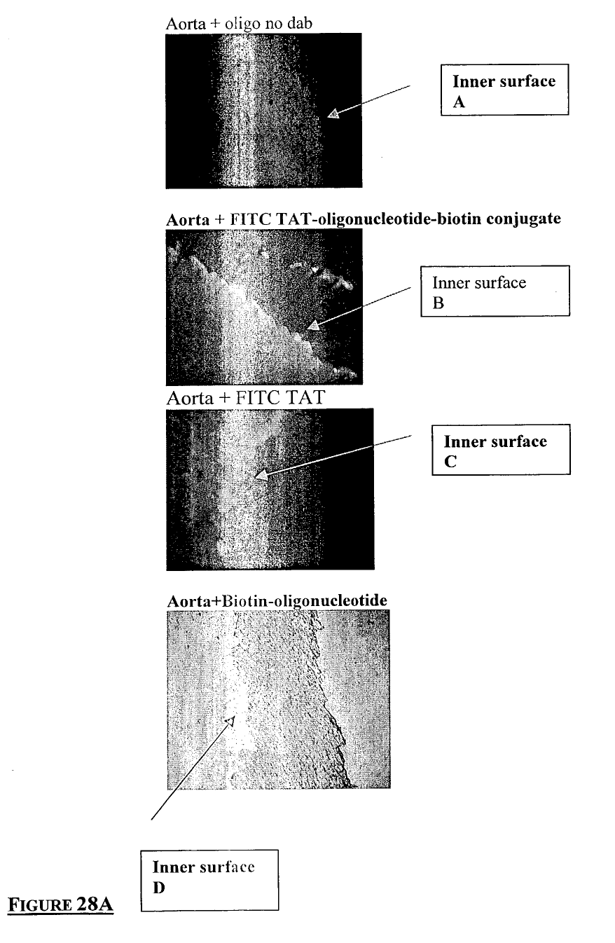

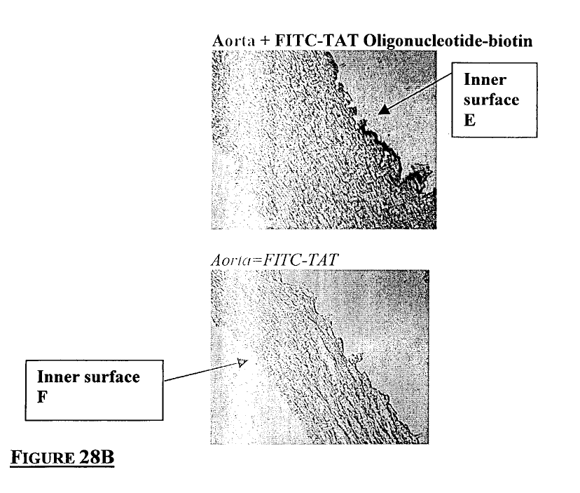

FIG. 27A demonstrates that a clear increase in % loaded cells coincides with administration of loaded vehicle into the animal.

In the control animal, to which no ultrasound is applied, a gradual decline in labelled cell number is observed. In contrast, the effect of ultrasound treatment on loaded cells in vivo is pronounced, and a dramatic decrease is shown following both 5 minute bursts of ultrasound treatment at 6 W/cm², pulsed wave; 35%.

FIG. 27B illustrates the ultrasound mediated localisation of FITC-labelled TAT in the treated kidney compared to the control organ. Less fluorescent staining is evident in the kidney to which no direct application of ultrasound is carried out. However, in the tissues which ultrasound is applied, strong fluorescence is evident, clearly exhibiting enhanced localisation and uptake at the treated area. This indicates that the peptide released at the site of ultrasound treatment, trafficks across cell membranes into neighbouring endothelial cells. This phenomenon may be exploited to enable uptake of payload conjugates by target tissues following ultrasound-mediated release from the erythrocyte vehicle.

### Example 27

### Uptake of TAT, Oligonucleotide and TAT-Oligonucleotide by the Lining of Blood Vessels

In this series of experiments the objective is to determine whether or not TAT, a candidate oligonucleotide (Scaggiante et al., Eur. J. Biochem., 252, 207-215) and a TAT-oligonucleotide conjugate are taken up by the inner lining of blood vessels.

In order to do so, TAT is labelled with FITC (fluorescein isothiocyanate) (Alta Biosciences UK), the oligonucleotide having a sequence 5′ TGT TTG TTT GTT TGT TTG TTT GTT TGT 3′ (MWG Biotech, UK) is labelled with biotin and the conjugate is co-labelled with FITC on the peptide and biotin on the oligonucleotide (Alta Biosciences, UK). Samples of each molecular species ([TAT]=200 μg/ml; [oligonucleotide]=50 μg/ml and [TAToligonucleotide conjugate]=250 μ/ml) are placed in contact with the inner surface of rabbit aorta at room temperature for 30min.

Following incubation, each section of aorta is washed 3 times in phosphate buffered saline and then placed in a vial containing 4% (w/v) paraformaldehyde solution. Paraffin wax sections of each sample are prepared and viewed directly using a fluorescent microscope to detect the presence of the peptide (FITC) either alone or as a molecular partner in the conjugate. Detection of the oligonucleotide, either alone or as a partner in the conjugate is accomplished by incubating sections in 0.3% H₂O₂ for 30 min. at room temperature. After rinsing for 10 min. in water, sections are incubated with Elite ABC complex (avidin-peroxidase) (Vectastain, U.S.A.) for 90 min at 37° C. Slides are washed in phosphate buffered saline for 3 min. and subsequently incubated in peroxidase substrate (3,3′-diaminobenzidine [DAB]) for 5 min. at room temperature. The reaction is terminated with water and sections are examined using a light microscopy.

Results 27

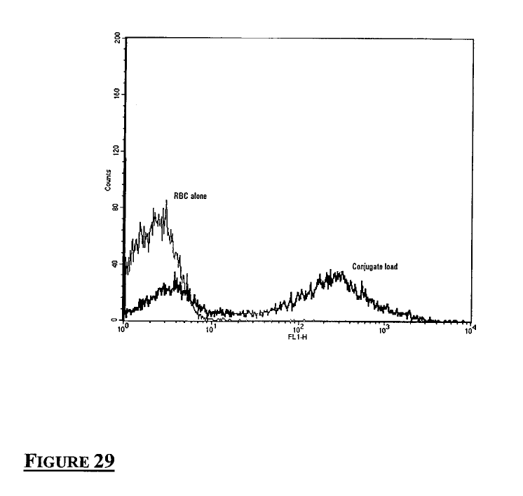

The data obtained from these experiments are shown in FIG. 28A and FIG. 28B. Clear fluorescent staining on the inner vessel surface is observed under fluorescence microscospy in aorta samples which are in contact with either TAT-oligonucleotide conjugate (FIG. 28A, Panel B) or TAT alone (FIG. 28A, Panel C). No fluorescence is detected in samples which are incubated with oligonucleotide alone (FIG. 28A, Panel A). These results demonstrate that both TAT and TAT-oligonucleotide are taken up by the inner lining of the aorta.

In addition, clear staining for biotin (the presence of oligonucleotide) is present on the inner vessel surface in samples which are in contact with oligonucleotide alone and TAT-oligonucleotide conjugate (FIG. 28A, Panel D & FIG. 28B Panel E, respectively). No staining is evident in sections that are in contact with TAT alone (FIG. 28B, Panel F). These results demonstrate that oligonucleotide is taken up by the inner lining of the vessel which are in contact with oligonucleotide alone or with TAT-oligonucleotide conjugate.

In overall terms the results demonstrate the co-existence of the TAT peptide and oligonucleotide partners in tissues placed in contact with the conjugate (FIG. 28A, Panel B & FIG. 28B, Panel E) and this demonstrates functionality of the peptide in terms of oligonucleotide carriage into tissue.

### Example 28

### Uptake of Oligonucleotide and TAT-Oligonucleotide Conjugate by the Inner Surface of Aorta Following Ultrasound-Mediated Release from Human Erythrocytes

In this series of experiments it is decided to load the oligonucleotide and the TAT-oligonucleotide into human erythrocytes. The loaded erythrocytes are then exposed to ultrasound and the resulting lysates placed in contact with the inner surface of a blood vessel.

Demonstrating uptake of the conjugate into the vessel inner lining by detecting the presence of both TAT and oligonucleotide partners indicates functionality of the TAT partner in terms of payload carriage to that tissue.

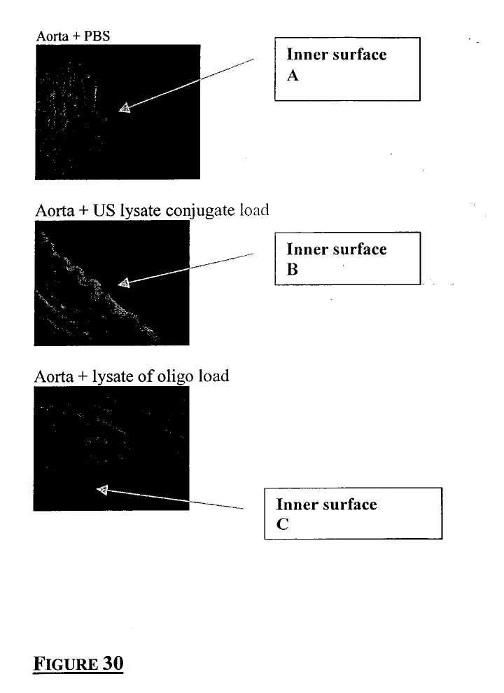

Human erythrocytes (7×10⁸ cells/ml in PBS) are sensitised and loaded with 1 mg/ml oligonucleotide (labelled with biotin) and TAT-oligonucleotide conjugate (labelled on the peptide with FITC and on the oligonucleotide with biotin) as described above. Following washing, loading of the conjugate is confirmed by flow cytometry as shown in FIG. 29. A shift in the population to the right indicates loading with the FITC label on the peptide partner of the conjugate. 100 μl aliquots of cells are exposed to ultrasound (3 W/cm², 36 seconds at 1 MHz) using the tissue mimicking system as described above. Lysates resulting from ultrasound treatment are recovered and incubated at room temperature together with the inner surface of rabbit aorta for 1 h. Following incubation, tissues are washed three times in PBS and samples are treated as described in the previous example above. Sections are examined using fluorescence microscopy to detect the presence of TAT. Sections of aorta are also stained for biotin (the presence of oligonucleotide) and examined using light microscopy as described above.

Results 28

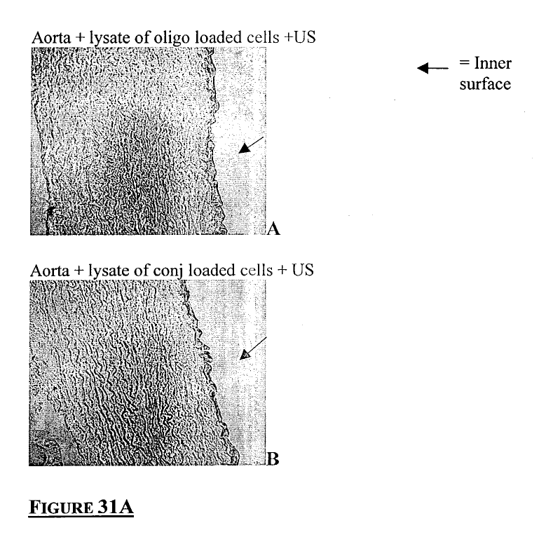

The results following examination of sections using fluorescence microscopy are shown in FIG. 30 and they demonstrate clear fluorescent staining on the inner surface of the vessel which is in contact with lysates from cells loaded with the conjugate (FIG. 30, Panel B). No fluorescent staining is observed in control tissue which is in contact with phosphate buffered saline (FIG. 30, Panel A) or in tissues which are in contact with oligonucleotide (Panel C). These results demonstrate that the TAT partner in the conjugate is taken up by the inner lining of the vessel. The results also demonstrate that the TAT exhibits functionality following ultrasound-mediated release from human erythrocytes.

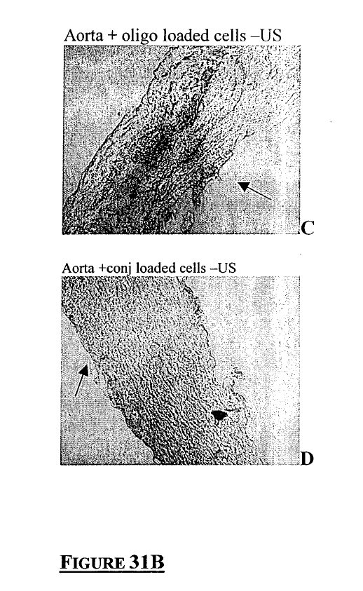

The results obtained following staining of the vessel samples for the presence of biotinylated oligonucleotide are shown in FIGS. 31A and 31B and they again show clear staining for oligonucleotide in samples of tissue which are in contact with both the oligonucleotide- and TAT-oligonucleotide conjugate-containing lysates (FIG. 31A, Panels A & B, respectively). In samples of tissue which are placed in contact with the intact vehicle, loaded with either oligonucleotide or conjugate, no staining for oligonucleotide is detected (FIGS. 31B, C & D, respectively). The results demonstrate that either oligonucleotide or conjugate, released from the loaded erythrocytes using ultrasound, is taken up by the tissues. They also confirm that the oligonucleotide co-resides with the TAT partner of the conjugate in tissues that are in contact with ultrasound-derived lysates of cells loaded with that conjugate. The results demonstrate that the TAT remains functional in terms of uptake by tissues following ultrasound-mediated release from the erythrocyte vehicle.

### Example 29

### Ultrasound-Mediated Deposition of TAT-Oligonucleotide Conjugate in Mouse Kidney in Vivo

In this series of experiments mouse erythrocytes are loaded with oligonucleotide (biotinylated) and TAT-oligonucleotide (FITC on the peptide and biotin on the oligonucleotide).

Recipient animals are anaesthetised using 2% isofluorane in a 2 L/min 02 carrier. The preparations are injected into recipient animals (50-200 μl) through the tail vein and allowed to circulate for 5 min. The kidney is surgically exposed through the abdomen and ultrasound gel is placed over the target kidney to mediate contact with the ultrasound head. Treatment consisted of exposing the target kidney to 1 MHz ultrasound at 6 W/cm² and using pulsed ultrasound at 35% continuous wave for 4 min. Following treatment, both treated and untreated kidneys are harvested from the animal and placed in a 4% paraformaldehyde solution. Paraffin wax sections are prepared as described above and sections are either observed directly for fluorescence (presence of the TAT partner) or stained for biotin (presence of oligonucleotide). The latter are then viewed using light microscopy.

Results 29

In both cases, the non-target kidney shows no staining for either fluorescent signal from the TAT partner in the conjugate or for biotinylated oligonucleotide, indicating a lack of deposition in the non-treated organ. In the treated organ from the animal receiving the oligonucleotide loaded erythrocytes, little or no staining is evident. However, in the treated organ from the animal injected with the conjugate, a clear fluorescent signal is evident and this indicated the presence of TAT. In addition, sections from this organ also exhibit a positive signal for biotin and this indicates the co-deposition of oligonucleotide in the tissues. The results demonstrate that treatment with ultrasound facilitates deposition of conjugate in the target organ and that conjugate is retained at the target side as a result of TAT functionality. The latter facilitates retention of oligonucleotide at the target and this is confirmed by an absence of oligonucleotide in treated kidney from the animal injected with oligonucleo-tide loaded erythrocytes.

### Example 30

### Systemic Release and Subsequent Localised Uptake of TAT Fragment in Liver Tissue

Objectives

The aim of this study is to investigate whether ultrasound mediates systemic release and subsequent localised uptake of TAT fragment in liver tissue. Due to its ability to translocate membranes, TAT-fragment is chosen for this study, as it is sure to be taken up at the site of release. Release and uptake are related to both blood flow at the site of release, and uptake kinetics of the released therapeutic agent by the surrounding cells. This is demonstrated in well vascularised tissue, i.e the right medial lobe of the liver.

Summary

Briefly, this study involves the sensitisation and loading of TAT-fragment-Fluorescein (TAT-FITC) into porcine erythrocytes. Following the preparation of the loaded vehicle, the cells are administered in vivo to the animal. Ultrasound is applied to the exposed liver of the animal. Venous samples are taken and histopathological samples collected from various regions of the liver, for determination of fluorescence.

Materials and Methods

Test Material (Payload)

Identity: TAT-FRAGMENT (FLUROSCEIN labelled)

AA Formula: Fluorescein-GRKKRRQRRRPPQC-amide

Receipt and Storage of Articles

Article Supply: 30 ml of blood is removed from each pig at the time of selection (PT1) and processed using the outlined procedure of Electrosensitisation and loading with TAT-FITC. Article Storage: The articles are stored under refrigerated conditions (2-8° C.). The test article is transferred to a hypodermic syringe and stored at room temperature for 10 minutes before administration.

Receipt and Storage of Test System

Test System Description: This comprises one healthy, physiologically mature pig of a crossbreed type (Large White×Landrace), of the male sex, at least four weeks of age and weighing between 9 and 12 kg at the time of selection.

Test System Storage: Storage of the Test System is carried out in a specific area of the holding facility. Handling, article introduction, treatment and subsequent sampling will all be carried out at licensed facilities.

Test System Termination: The test system is euthanised at the termination of all sampling procedures by intravenous injection of up to 150 mg pentobarbitone/Kg bodyweight, equivalent to 1 mL Euthatal (Merial)/ 1.3 Kg bodyweight or 1 mL Sagatal (Merial)/ 0.4 Kg bodyweight

Receipt and Storage of Samples

Timepoints: Blood samples are taken from the test system as detailed below.

| | | Time | | |
|---|---|---|---|---|
| | | (min after | | |
| | | article | | |
| | Time | admin- | Sample | |
| Day | reference | istration) | FACSCAN | Note |
| −1 | PT1 | | | Bled @ ARINI |
| 0 | PT2 | | | |
| | PT3 | −5 | * | |
| | T1 | 0 | * | Article administration |
| | T2 | 5 | * | |
| | T3 | 10 | * | |
| | T4 | 15 | * | |
| | T5 | 20 | * | |
| | T6 | 30 | * | ultrasound on liver starts |
| | | | | (1 × 2.4 min) |
| | T7 | 32.5 | * | ultrasound ends |
| | T8 | 35 | * | ultrasound on liver starts |
| | | | | (1 × 2.5 min) |
| | T9 | 37.5 | * | ultrasound ends |
| | T10 | 40 | * | ultrasound on liver starts |
| | | | | (1 × 2.5 min) |
| | T11 | 42.5 | * | ultrasound ends |
| | T12 | 45 | * | ultrasound on liver starts |
| | | | | (1 × 5 min) |
| | T13 | 50 | * | ultrasound ends |
| | T14 | 55 | * | ultrasound on liver starts |
| | | | | (1 × 5 min) |
| | T15 | 60 | * | ultrasound ends |
| | T16 | 65 | * | ultrasound on liver starts |
| | | | | (1 × 5 min) |
| | T17 | 70 | * | ultrasound ends |
| | T18 | 75 | * | ultrasound on liver starts |
| | | | | (1 × 5 min) |
| | T19 | 80 | * | ultrasound ends |
| | T20 | 85 | * | ultrasound on liver starts |
| | | | | (1 × 10 min) |
| | T21 | 95 | * | ultrasound ends |
| | T22 | 100 | * | ultrasound on liver starts |
| | | | | (1 × 10 min) |
| | T23 | 110 | * | ultrasound ends |
| | T24 | 115 | * | ultrasound on liver starts |
| | | | | (1 × 20 min) |
| | T25 | 125 | * | |
| | T26 | 135 | * | ultrasound ends |

Method for Sample Analysis

Volume: A minimum of 0.5 mL blood samples (actual volume recorded) is taken at each timepoint into heparininsed containers, labelled with the protocol number, date, animal identity and timepoint reference. At detailed timepoints an additional blood sample of at least 3 mL is taken at each timepoint into heparinised containers, labelled with the protocol number, date, animal identity and timepoint reference. Where catheters are being used a minimum of 0.25 mL blood is withdrawn and discarded before each sample is taken and a minimum of 1 mL heparinised saline is infused.

Processing: Each sample is processed as follows: One aliquot of 10 μL blood is diluted with 500 μL PBS, the remainder is maintained as whole blood in plastic heparinised containers, labelled with the protocol number, animal identity and time reference.

Storage: Blood samples is stored under refrigerated conditions (2-8° C.) until assayed.

Analysis: Processed blood samples is analysed using FACS analysis.

Ultrasound Treatment

Area: The dorsal portion of the right lateral lobe of the exposed liver is targeted with ultrasound. The treated area is marked with a suture needle between treatments and for further histopathogical analysis.

Preparation of treatment area: The surface of this area is liberally covered with Alpha Lube gel (Ultrasonic Scanning Gel, BCF Technology Limited) before application of the head.

Exposure to Ultrasound: This consists of period of exposure to ultrasound using a RICH-MAR CRM-1 machine fitted with a 1 mHz head, starting at 30 minutes after administration of the article, with 3 treatments at 2.5 minute intervals, 4 treatments at 5 minute intervals and 2 treatments at 10 minute intervals at a power setting of 6.0 watts/cm² at 35% pulsed wave. The duration of ultrasound for the four treatments is 2.5 minutes, for the second treatment it is for 5 minutes, for the third, 10 minutes and a final treatment of 20 minutes.

Histopathological Samples

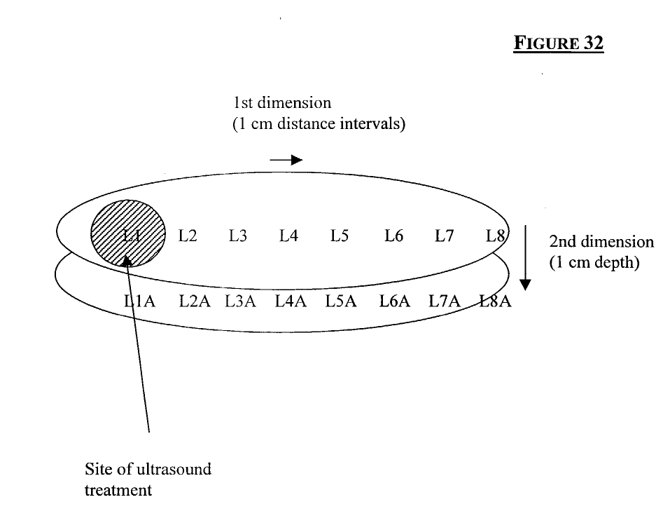

Tissue samples: Immediately following termination of the test system, 2 mm tissue samples (at 1 cm intervals from the treated area) are excised from the right medial lobe of the liver, as shown in FIG. 32 below. The circle denotes the area of ultrasound treatment and the numbers illustrate where the tissue samples are collected. Corresponding samples from the right lateral lobe are used as a control. Samples are labelled L1, L2 etc.

In addition, tissue samples are collected directly under the site of ultrasound treatment and at 0.5 cm depths into the organ, labelled L1A, L1B etc to enable a 2 dimensional profile of localised release.

Sample storage: Upon removal from the animal, each tissue sample is placed in 3 mL 5% paraformaldehyde as described.

Sample processing: Tissue samples are paraffin wax embedded for visualisation using the fluorescent microscope.

Sample analysis: Visual observation of the quantity of fluorescence in the various samples is used as a qualitative measure of localised release of TAT-FITC.

Results

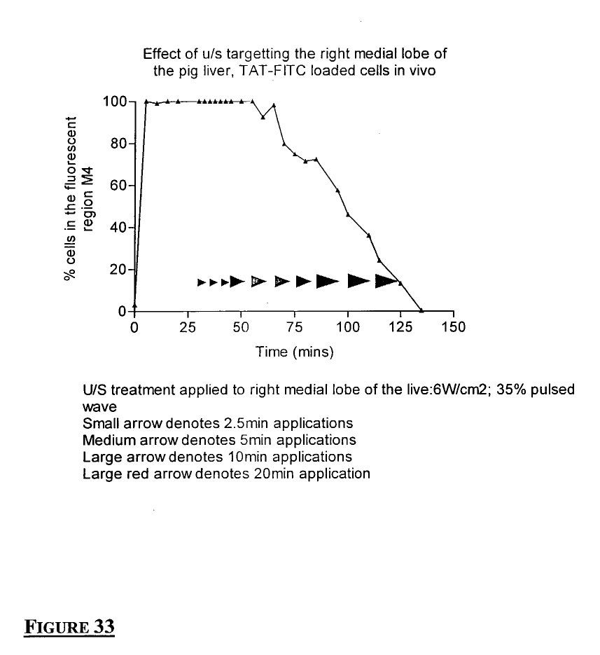

FIG. 33 shows systemic levels of loaded cells, as determined by FACS analysis. Following the administration of article to the animal, stability of loaded vehicle in vivo appeared to be broadly reached prior to the application of ultrasound. During the 3×2.5 min applications of treatment, there appear to be negligible effects. A decline in loaded cell number appears during the 2^{nd} of the 5-minute applications and a fairly steady effect is observed during the 5 and 10-minute applications. This effect continues until the end of the experiment, where loaded cells can no longer be detected in circulation.

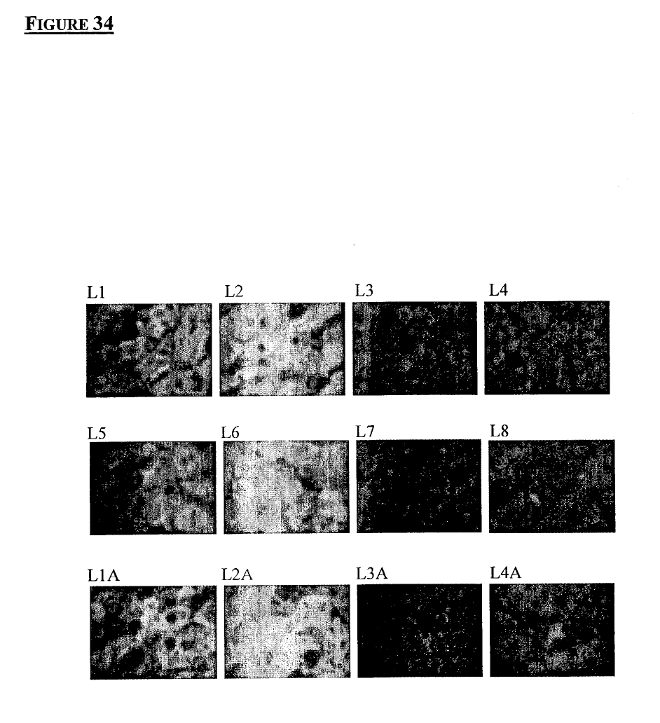

FIG. 34 shows the histopathological analysis on the ultrasound treated right medial lobe of the liver. The uptake of any TAT-FITC is illustrated by fluorescence associated with the liver tissue and this is purely qualitative. On this basis, the uptake of TAT-FITC is observed in samples L1, where the ultrasound probe is directly applied, and L1A, 1 cm below the surface. TAT-FITC is also present in L2, which is 1 cm along the right medial lobe surface, from the application of ultrasound. However, no TAT-FITC appears to be present in any other sample taken from the right medial lobe.

Broadly, the results indicate that ultrasound treatment applied to a specific area results in the release (as shown by the FACS data) and the uptake ( as shown by the histopathological data) of the carrier peptide, TAT-fragment-FITC. Uptake is confined to the area directly under the ultrasound probe, 1 cm below that, and 1 cm in one direction along the surface of the lobe.

Conclusion

The aim of this experiment is to illustrate that release and uptake of this carrier peptide could occur at a well vascularised site, such as the liver. The results show that at this site, blood flow is sufficiently slow to allow uptake of the therapeutic agent into the hepatic cells in close proximity to the site of release, i.e within a 1 cm radius from the point of treatment.

This result can direct further research in one of two directions—either to assess the release and localisation of uptake of carrier peptide at a site where there is faster blood flow, to investigate what is the flow required to enable uptake at the site of release.

In addition, it would also be useful to load a therapeutic agent, which does not have membrane translocation ability of TAT-fragment, but for example receptor binding ability. This would delineate if the slow blood flow rate at the liver can allow time for receptor binding, and not membrane translocation. Such questions would enable an assessment of the localisation of delivery of the technology.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of sensitising a red blood cell to ultrasound, comprising exposing the red blood cell to an electric field.

2. A method of sensitizing a red blood cell to tultrasound, comprising the steps of:
providing a red blood cell and subjecting the red blood cell to an electric field, the electric field having sufficient energy to electrosensitise the cell.

3. The method of claim 2, in which said red blood cell sensitised using electric field pulsing may be selectively disrupted using ultrasound.

4. A method of selectively disrupting a red blood cell, the method comprising the steps of:
(a) providing a red blood cell;
(b) electrosensitising said red blood cell; and
(c) disrupting said red blood cell by subjecting said red blood cell to ultrasound.

5. The method of claim 2 or claim 4, in which the electrosensitisation comprises the step of applying an electric pulse to a red blood cell.

6. The method of claim 5, in which the electric pulse is from about 0.1 kVolts/cm to about 10 kVolts/cm under in vitro conditions.

7. The method of claim 2 or claim 4, further comprising the step of loading the red blood cell with an agent.

8. The method of claim 7, in which the sensitisation of the red blood cell precedes the loading of the agent.

9. The method of claim 7, in which the loading of the agent precedes the sensitisation of the red blood cell.

10. The method of claim 7, in which the sensitisation of the red blood cell and the loading of the agent are substantially simultaneous.

11. A method for selectively releasing an agent from a red blood cell comprising the steps of:
(a) loading a red blood cell with an agent;
(b) electrosensitising the red blood cell; and
(c) causing the agent to be released from the sensitised red blood cell by applying ultrasound at a frequency and energy sufficient to cause disruption of the red blood cell but insufficient to cause disruption of unsensitised red blood cells.

12. The method of claim 11, in which the electrosensitisation procedure is an in vitro or ex-vivo procedure.

13. The method of claim 11 or claim 12, in which the electrosensitisation comprises the step of applying an electric field to a red blood cell.

14. The method of claim 13, in which the electric pulse is from about 0.1 kVolts/cm to about 10 kVolts/cm under in vitro conditions.

15. The method of claim 13, in which the electric pulse is applied for between 1 μs and 100 milliseconds.

16. The method of claim 4 or claim 11, in which the ultrasound is selected from the group consisting of diagnostic ultrasound, therapeutic ultrasound and a combination of diagnostic and therapeutic ultrasound.

17. The method of claim 16, in which the applied ultrasound energy source is at a power level of from about 0.05 W/cm² to about 100 W/cm².

18. A method for delivering an agent to a target site in a vertebrate, comprising the steps of:
(a) loading a red blood cell with an agent;
(b) electrosensitising the red blood cell;
(c) introducing the red blood cell into a vertebrate; and
(d) causing the agent to be released from the sensitised red blood cell by applying ultrasound at a frequency and energy sufficient to cause disruption of the red blood cell but insufficient to cause disruption of unsensitised red blood cells.

19. The method of claim 18, in which the red blood cell is PEGylated prior to being introduced into the vertebrate.

20. The method of claim 18 or claim 19, in which the vertebrate is a mammal.

21. The method of claim 11 or claim 18, in which the loading of the agent is substantially simultaneous with the sensitisation of the red blood cell.

22. The method of claim 11 or claim 18, in which the sensitisation of the red blood cell precedes the loading of the agent.

23. The method of claim 11 or claim 18, in which the loading of the agent precedes the sensitisation of the red blood cell.

24. The method of claim 11 or 18, in which the loading is performed by a procedure selected from a group consisting of electroporation, sonoporation, microinjection, membrane intercalation, microparticle bombardment, lipid-mediated transfection, viral infection, osmosis, osmotic pulsing, diffusion, endocytosis, modifying the thermal, ionic and/pH environment of the red blood cell, applying electromagnetic radiation to the red blood cell and crosslinking to a red blood cell surface component.

25. The method of claim 11 or claim 18, in which the agent is selected from a group consisting of a biologically active molecule, a protein, a polypeptide, a peptide, a nucleic acid, a virus, a virus-like particle, a nucleotide, a ribonucleotide, a deoxyribonucleotide, a modified deoxyribonucleotide, a heteroduplex, a nanoparticle, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a carbohydrate, and mixtures, fusions, combinations or conjugates of the above.

26. The method of claim 25, in which the agent is conjugated to, fused to, mixed with or combined with an imaging agent.

27. A kit comprising a red blood cell, an agent, packaging materials therefor and instructions for use comprising the steps of:
(a) electrosensitising a red blood cell;
(b) loading the red blood cell with an agent; and
(c) causing the agent to be released from the sensitised red blood cell by applying ultrasound at a frequency and energy sufficient to cause disruption of the red blood cell but insufficient to cause disruption of unsensitised red blood cells.

28. A kit comprising a red blood cell which is loaded with an agent, packaging materials therefor and instructions for use comprising the steps of:
(a) electrosensitising a red blood cell; and
(b) causing the agent to be released from the sensitised red blood cell by applying ultrasound at a frequency and energy to cause disruption of the sensitised red blood cell but insufficient to cause disruption of unsensitised red blood cells.

29. A kit comprising a sensitised red blood cell loaded with an agent which has been sensitised using ultrasound and instructions for use comprising the steps of:
(a) causing the agent to be released from the sensitised red blood cell by applying ultrasound at a frequency and energy to cause disruption of the sensitised red blood cell but insufficient to cause disruption of unsensitised red blood cells.

30. The kit of claims 27, 28 or 29, in which the kit further comprises polyethylene glycol.

31. The kit of claims 27, 28 or 29, in which the kit further comprises a liquid selected from the group consisting of a buffer, diluent or other excipient.

32. The kit of claim 31, in which the liquid is selected from the group consisting of a saline buffer, a physiological buffer and plasma.

33. A red blood cell composition made by the method of claims 2, 4, 11 or 18.
